(19) United States
(12) Patent Application Publication
(10) Pub. No.: US 2026/0061175 A1
(43) Pub. Date: Mar. 5, 2026

(54) HEMOSTASIS SEAL STACK

(71) Applicant: EDWARDS LIFESCIENCES CORPORATION, Irvine, CA (US)

(72) Inventors: Nithya Sridhar, Irvine, CA (US); Joshua Thomas Derr, Irvine, CA (US); Thomas Andrew Sunding, Costa Mesa, CA (US); Kristen Hicks, Irvine, CA (US); Brian James Thompson, Laguna Niguel, CA (US); John Richard Carpenter, Irvine, CA (US); Evan Matthew Dowey, Lake Forest, CA (US); James Adam Krisher, Lake Forest, CA (US); William Willis, JR., Costa Mesa, CA (US); Hien T. Nguyen, Aliso Viejo, CA (US); Cody Samuel Temple, Ladera Ranch, CA (US); Tyler Lindsey Price, Westminster, CA (US)

(21) Appl. No.: 19/381,670

(22) Filed: Nov. 6, 2025

Related U.S. Application Data
(63) Continuation of application No. PCT/US2024/028571, filed on May 9, 2024.
(60) Provisional application No. 63/501,127, filed on May 9, 2023.

(51) Int. Cl. A61M 39/06

(52) U.S. Cl. CPC A61M 39/0693; A61M 2039/064

(57) ABSTRACT

A cross-plane seal having a distal end and a proximal end includes a disc at the distal end. The disc includes a distal face at a distal side, a proximal face at a proximal side, a plurality of cut outs in the distal face forming a raised X-shape on the distal face, a first slit having a partial through-cut into the distal face and aligned with a first leg of the raised X-shape, the through-cut extending more than halfway through the thickness of the disc, a second slit having a partial through-cut into the proximal face and aligned with a second leg of the raised X-shape, the through-cut extending more than halfway through the thickness of the disc, and an intersection formed by the first slit and the second slit. An annular portion is connected to and extends proximally away from the disc.

### CROSS-REFERENCE TO RELATED APPLICATIONS

This application is a continuation of International Application No. PCT/US2024/028571, filed May 9, 2024, and entitled “HAEMOSTASIS SEAL STACK,” which claims the benefit of U.S. Provisional Application No. 63/501,127, filed May 9, 2023, and entitled “SEAL STACK,” the disclosure of which are hereby incorporated by reference in its entirety.

### BACKGROUND

The present disclosure relates to seals, and in particular, to seals for a guide sheath assembly.

A guide sheath assembly, or introducer sheath assembly, is used to introduce a medical device into the body of a patient and guide the device to a desired location within the body. When a guide sheath assembly is required, an incision is made into the patient so that a portion of the guide sheath assembly can be inserted into the patient though the incision. As such, the guide sheath assembly provides an opening into the body where potential blood loss, or the loss of hemostasis, can occur. A seal stack for the guide sheath assembly helps to prevent blood loss from the patient while the guide sheath assembly is inserted.

### SUMMARY

A cross-plane seal having a distal end and a proximal end includes a disc at the distal end of the cross-plane seal. The disc includes a distal face at a distal side of the disc, a proximal face at a proximal side of the disc, a plurality of cut outs in the distal face of the disc forming a raised X-shape on the distal face of the disc, a first slit having a partial through-cut into the distal face of the disc and aligned with a first leg of the raised X-shape, the through-cut extending more than halfway through the thickness of the disc, a second slit having a partial through-cut into the proximal face of the disc and aligned with a second leg of the raised X-shape, the through-cut extending more than halfway through the thickness of the disc, and an intersection formed by the first slit and the second slit. The first slit is perpendicular to the second slit. An annular portion is connected to and extends proximally away from the disc to the proximal end of the cross-plane seal.

A seal stack for a seal hub of a guide sheath assembly includes a cross-plane seal having a distal end and a proximal end; a ring adjacent the cross-plane seal; a disc seal adjacent the ring; and a cap adjacent the disc seal. The cross-plane seal includes a disc at the distal end of the cross-plane seal, the disc including a distal face at a distal side of the disc; a proximal face at a proximal side of the disc; a plurality of cut outs in the distal face of the disc forming a raised X-shape on the distal face of the disc; a first slit having a partial through-cut into the distal face of the disc and aligned with a first leg of the raised X-shape, the through-cut extending more than halfway through the thickness of the disc; a second slit having a partial through-cut into the proximal face of the disc and aligned with a second leg of the raised X-shape, the through-cut extending more than halfway through the thickness of the disc; and an intersection formed by the first slit and the second slit. An annular portion is connected to and extends proximally away from the disc to the proximal end of the cross-plane seal.

A self-centering seal having a distal end and a proximal end includes a disc, a central planar region connected to an inner diameter of the disc, and a hole extending through the central planar region. The disc includes a plurality of raised curves extending axially from the distal end of the self-centering seal to the proximal end of the self-centering seal.

A seal stack for a seal hub of a guide sheath assembly includes a self-centering seal having a distal end and a proximal end, a ring adjacent to the self-centering seal, a disc seal adjacent to the ring, and a cap adjacent to the disc seal. The self-centering seal includes a disc, a central planar region connected to an inner diameter of the disc, and a hole extending through the central planar region The disc includes a plurality of raised curves extending axially from the distal end of the self-centering seal to the proximal end of the self-centering seal.

A cross-plane seal having a first end and a second includes a disc at the first end of the cross-plane seal and an annular portion connected to and extending away from the disc. The disc includes a first face at a first side of the disc, a second face at a second side of the disc, a star-shaped cut out having a star-shaped recess in the first face, and a first slit having a partial through-cut into the first face of the disc and aligned with arms of the star-shaped cut out, the through-cut extending more than halfway through a thickness of the disc. The disc further includes a second slit having a partial through-cut into the second face of the disc and aligned with arms of the star-shaped cut out, the through-cut extending more than halfway through the thickness of the disc. The first slit is perpendicular to the second slit. The disc further includes an intersection formed by the first slit and the second slit.

A funnel seal includes a proximal end, a distal end opposite the proximal end, a disc at the proximal end of the funnel seal, a proximal face of the disc forming a proximal face of the funnel seal, an annular portion connected to the disc, and a funnel portion forming a funnel-shaped opening that extends from the proximal end to the distal end.

A cross-plane seal having a proximal end and a distal end includes a disc at the proximal end of the cross-plane seal and an annular portion connected to and extending away from the disc. The disc includes a proximal face at a proximal side of the disc, a distal face at a distal side of the disc, and a plurality of first curved slits, each having a partial through-cut into the proximal face of the disc, the through-cut extending more than halfway through a thickness of the disc. The disc further includes a plurality of second curved slits, each having a partial through-cut into the proximal face of the disc, the through-cut extending more than halfway through the thickness of the disc. The first plurality of curved slits are offset from the second plurality of curved slits. The disc further includes an intersection formed by the first plurality of curved slits and the second plurality of curved slits.

A cinched seal having a proximal end and a distal end includes a proximal flange at the proximal end of the cinched seal, a distal flange at the distal end of the cinched seal, a proximal conical portion connected to and extending from the proximal flange, a proximal conical space within the proximal conical portion, a distal conical portion connected to and extending from the distal flange, a distal conical space within the distal conical portion, and a center portion connected to the proximal conical portion and the distal conical portion. The center portion has a C-shaped cross-section. The cinched seal further includes a hole extending through the center portion and fluidly connected to the proximal conical space and the distal conical space, a proximal coil positioned on the proximal conical portion adjacent the center portion, and a distal coil positioned on the distal conical portion adjacent the center portion.

A collapsible seal having a proximal end and a distal end includes a first ring defining the proximal end of the collapsible seal, a first membrane connected to the first ring, a second ring connected to the first membrane such that the first membrane is between the first ring and the second ring, a first wall extending across the first ring, a second wall extending across the second ring, a first hole extending through a center of the first wall, and a second hole extending through a center of the second wall.

### BRIEF DESCRIPTION OF THE DRAWINGS

FIG. 1 is a side view of a seal hub of a guide sheath assembly showing a seal stack within the seal hub.

FIG. 2A is a cross-sectional view of the seal hub of the guide sheath assembly showing the seal stack assembled within the seal hub.

FIG. 2B is a disassembled view of the seal hub of the guide sheath assembly showing the seal stack disassembled.

FIG. 3 is an exploded perspective view of the seal stack.

FIG. 4 is a perspective view of the cross-plane seal from a proximal end of the cross-plane seal.

FIG. 5A is a perspective view of the cross-plane seal from a distal end of the cross-plane seal.

FIG. 5B is a perspective view of the cross-plane seal from the distal end of the cross-plane seal showing the intersection between a partial through-cut first slit and a partial through-cut second slit when the cross-plane seal is deformed.

FIG. 6A is a cross-sectional view of a seal hub of a second example of a guide sheath assembly showing a seal stack assembled within the seal hub.

FIG. 6B is a disassembled view of the seal hub of the second example of the guide sheath assembly showing the seal stack disassembled.

FIG. 7 is side view of a seal hub of a third embodiment of a guide sheath assembly showing a seal stack within the seal hub.

FIG. 8 is an exploded view of the seal hub of the third embodiment of the guide sheath assembly showing the seal stack with a self-centering seal.

FIG. 9 is a cross-sectional view of the seal hub of the third embodiment of the guide sheath assembly showing the seal stack assembled within the seal hub.

FIG. 10A is a perspective view of the self-centering seal from a proximal end of the self-centering seal.

FIG. 10B is a perspective view of the self-centering seal from a distal end of the self-centering seal.

FIG. 11 is a front plan view of the self-centering seal.

FIG. 12 is a cross-sectional side view of the self-centering seal.

FIG. 13A is a perspective view of a second embodiment of a self-centering seal from a proximal end of the second embodiment of the self-centering seal.

FIG. 13B is a perspective view of the second embodiment of the self-centering seal from a distal end of the second embodiment of the self-centering seal.

FIG. 13C is a front plan view of the second embodiment of the self-centering seal.

FIG. 13D is a cross-sectional side view of the second embodiment of the self-centering seal.

FIG. 14 is a cross-sectional side view of a third embodiment of the self-centering seal.

FIG. 15A is an orthogonal view of a second example of a cross-plane seal from a first end of the cross-plane seal.

FIG. 15B is an end view of the second example of the cross-plane seal from the first end of the cross-plane seal.

FIG. 15C is an enlarged end view of the first example of the cross-plane seal from the first end of the cross-plane seal showing the hole of the cross-plane seal.

FIG. 15D is an end view of the first example of the cross-plane seal from the second end of the cross-plane seal.

FIG. 16A is an orthogonal view of a third example of a cross-plane seal from a second end of the cross-plane seal.

FIG. 16B is an end view of the third example of the cross-plane seal from the second end of the cross-plane seal.

FIG. 16C is an end view of the third example of the cross-plane seal from the first end of the cross-plane seal.

FIG. 17A is a perspective view of a first example of a funnel seal from the proximal end of the funnel seal.

FIG. 17B is a cross-sectional side view of the first example of the funnel seal.

FIG. 18 is a cross-sectional side view of a second example of a funnel seal.

FIG. 19A is a perspective view of a third example of a funnel seal from the distal end showing curved ribs of the funnel seal.

FIG. 19B is an end view of the third example of the funnel seal from the distal end of the funnel seal showing the curved ribs of the funnel seal.

FIG. 19C is a perspective view of the third example of the funnel seal from the proximal end of the funnel seal.

FIG. 19D is an end view of the third example of the funnel seal from the proximal end of the funnel seal.

FIG. 19E is a cross-sectional side view of the third example of the funnel seal.

FIG. 20A is an end view of a first example of a curved cross-plane seal from a proximal end of the curved cross-plane seal.

FIG. 20B is a perspective view of the first example of the curved cross-plane seal from a distal end of the curved cross-plane seal.

FIG. 21A is an end view of a second example of a curved cross-plane seal from a proximal end of the curved cross-plane seal showing beveled first curved slits and beveled second curved slits.

FIG. 21B is a perspective view of the second example of the curved cross-plane seal from a distal end of the curved cross-plane seal.

FIG. 22A is a side view of a cinched seal.

FIG. 22B is a cross-sectional side view of the cinched seal.

FIG. 23A is an isometric view of a collapsible seal in a collapsed position.

FIG. 23B is a side view of the collapsible seal in the collapsed position.

FIG. 23C is a side view of the collapsible seal in an expanded position.

FIG. 23D is a cross-sectional side view of a seal hub with the collapsible seal when no device is inserted into the seal hub.

FIG. 23E is a cross-sectional side view of the seal hub with the collapsible seal when a device is inserted into the seal hub.

FIG. 24 is a cross-sectional side view of a second embodiment of a seal hub with a second embodiment of a collapsible seal when a device is inserted into a seal hub.

### DETAILED DESCRIPTION

In general, the present disclosure describes a seal stack for a seal hub of a guide sheath assembly that includes (1) a duckbill seal or a cross-slit seal, (2) a cross-plane seal, (3) a ring, (4) a disc seal, and (5) a cap. The cross-plane seal includes a disc connected to an annular portion. The cross-plane seal has cut outs in the distal face of the disc of the cross-plane seal to form a raised X-shape on the disc of the cross-plane seal. A first partial through-cut slit is aligned with a first leg of the X-shape on the distal face of the disc of the cross-plane seal, and a second partial through-cut slit is aligned with a second leg of the X-shape on the proximal face of the disc of the cross-plane seal. The first slit and the second slit are perpendicular to each other on opposite planes and extend more than halfway through the thickness of the disc of the cross-plane seal such that they partially intersect. The cross-plane seal allows for insertion and retention of a medical device, either coaxially or non-coaxially, while sealing against the medical device to minimize hemostasis loss. The cross-plane seal also maintains hemostasis when no devices are inserted through the cross-plane seal.

The present disclosure also describes a self-centering seal having a disc surrounding a central planar region, the disc having a plurality of raised curves that are flexible to expand and contract for improving sealing during off-axis insertion and retention of a medical device.

The present disclosure also describes a cross-plane seal having a star-shaped cut out on a disc that is connected to an annular portion. A first partial through-cut slit is aligned with arms of the star-shaped cut out on a first face of the disc, and a second partial through-cut slit is aligned with arms of the star-shaped cut out on a second face of the disc. The first slit and the second slit are perpendicular to each other on opposite planes and extend more than halfway through the thickness of the disc of the cross-plane seal so that they partially intersect to form a hole. The star-shaped cut out provides a visual target for device insertion through the cross-plane seal, reducing the likelihood of tears. The star-shaped cut out also allows for insertion of both small and large devices through the cross-plane seal while minimizing hemostasis loss.

The present disclosure also describes a funnel seal having a funnel-shaped space extending from a proximal end to a distal end, providing a visual guide for device insertion through a center of the funnel-shaped seal, reducing the likelihood of seal tears. The funnel-shaped seal provides support for an inserted device, allowing the funnel seal to be used with both large and small devices, and provides additional sealing surface area. The funnel-shaped seal may have a ring portion to provide additional support, particularly for the insertion of smaller devices. Further, the funnel-shaped seal may have curved ribs to provide support and center the device while also allowing for evenly distributed circumferential buckling and additional sealing during the insertion of larger devices.

The present disclosure also describes a curved cross-plane seal having a disc and an annular portion with first curved partial through-cut slits extending from a distal face of the disc, and second curved partial through-cut slits extending from a proximal face of the disc. The first curved slits are curved in a first direction and the second curved slits are curved in a second direction opposite the first direction. The first curved slits and the second curved slits extend more than halfway through the thickness of the disc of the cross-plane seal so that they partially intersect to form a hole. The curved slits provide support for inserted devices and mitigated drifting of inserted devices. The curved slits may have a beveled edge to provide a guide for device insertion.

The present disclosure also describes a cinched seal having a proximal conical portion connected to a center portion, which is connected to a distal conical portion, the proximal conical portion guiding a medical device to a hole within the center portion during insertion and the distal conical portion guiding the medical device to the hole within the center portion during retraction. The cinched seal also includes a proximal coil and a distal coil to provide compression forces to the center portion, cinching the center portion closed when a medical device is retracted from the cinched seal. As a result, the medical device is supported and centered within the cinched seal, and the cinched seal can accommodate medical devices of various sizes while maintaining hemostasis.

The present disclosure also describes a collapsible seal that comprises rings connected together via one or more membranes such that the collapsible seal can expand upon insertion of a medical device and retract upon retraction of the medical device, providing a single seal to maintain hemostasis.

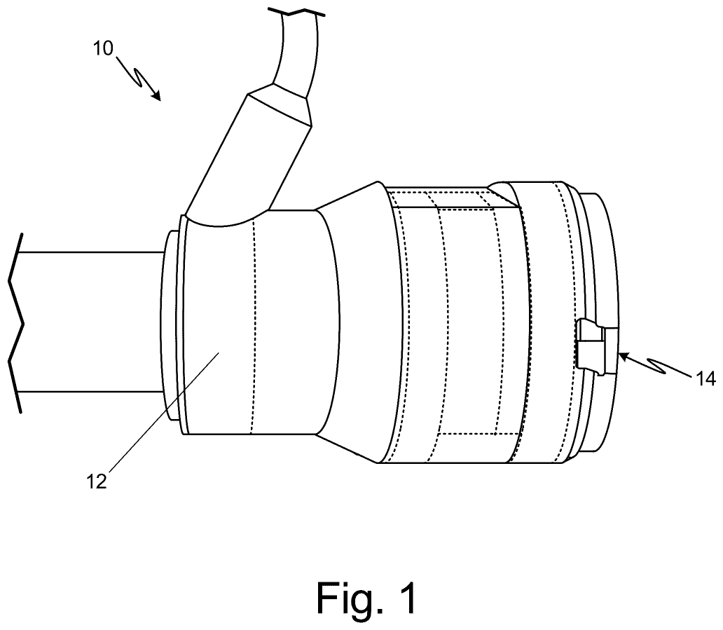

FIG. 1 is a side view of seal hub 12 of guide sheath assembly 10 showing seal stack 14 within seal hub 12. Guide sheath assembly 10 includes seal hub 12 and seal stack 14.

Guide sheath assembly 10 is configured to introduce a medical device into the body of a patient. A distal end of guide sheath assembly 10 is configured to be inserted into the body of the patient. A proximal end of guide sheath assembly 10 is configured to be outside of the body of the patient and is accessible by a healthcare worker. Seal hub 12 is at the proximal end of guide sheath assembly 10. Seal hub 12 may be housed within a handle, which may also be configured to flush and aspirate. Seal hub 12 accepts insertion of a medical device through seal stack 14 from a proximal end of seal hub 12. Seal hub 12 of guide sheath assembly 10 provides a housing for seal stack 14. As such, seal stack 14 fits within seal hub 12 of guide sheath assembly 10. Seal stack 14 comprises a plurality of seals, which are assembled within seal hub 12. Seal stack 14 accepts insertion of a medical device through a proximal end of seal stack 14 such that seal stack 14 surrounds a portion of the medical device.

An incision is made into the body of a patient to insert guide sheath assembly 10 into the patient. For example, an incision can be made into the neck of a patient such that guide sheath assembly 10 has jugular access into the body of the patient. A distal portion of guide sheath assembly 10 is inserted into the body of the patient through the incision. A medical device, such as a guidewire, a loader, an introducer, or any other suitable device, can be inserted into the body of the patient through guide sheath assembly 10. As such, guide sheath assembly 10 provides a port into the body of the patient. To maintain hemostasis by preventing blood loss through guide sheath assembly 10, seal stack 14 is included in seal hub 12 of guide sheath assembly 10. Seal stack 14 can provide a seal when nothing is inserted in guide sheath assembly 10. Seal stack 14 can also provide a seal when a medical device, such as the guidewire, the loader, the introducer, or any other device, is inserted into guide sheath assembly 10. Seal stack 14 seals on the guidewire, the loader, or any other medical device inserted into guide sheath assembly 10 to prevent fluid loss, such as blood loss. Seal stack 14 seals around the inserted medical device and retains the position of the medical device within seal stack 14.

Seal stack 14 in seal hub 12 of guide sheath assembly 10 helps prevent blood loss from the patient while guide sheath assembly 10 is inserted into the patient. Seal stack 14 in seal hub 12 provides a seal when nothing is present in guide sheath assembly 10 and when a medical device is inserted into guide sheath assembly 10. As such, seal stack 14 prevents hemostasis loss, which reduces the risk of cardiogenic shock caused by a sustained loss of hemostasis. As a result, the amount of blood transfusions needed for the patient is reduced and healthcare worker satisfaction is increased.

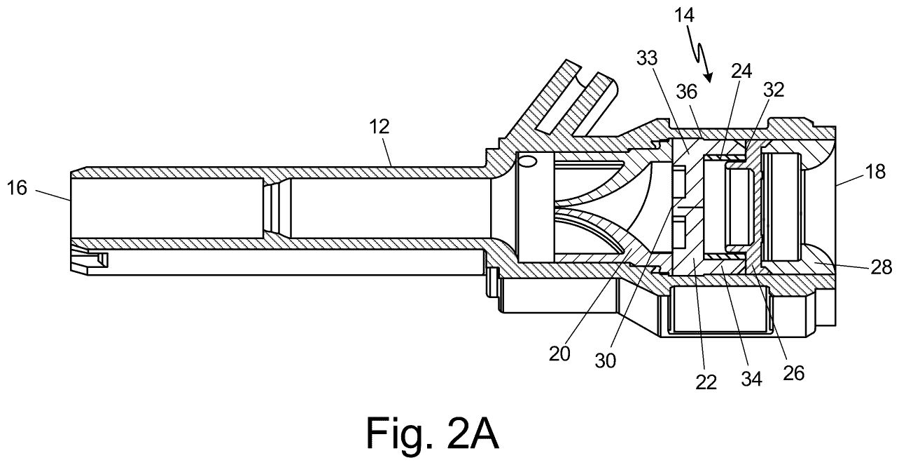

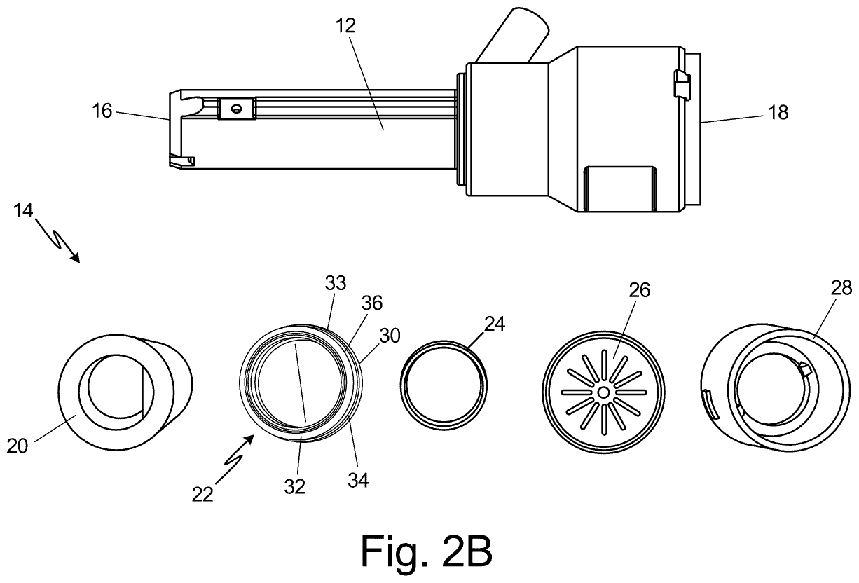

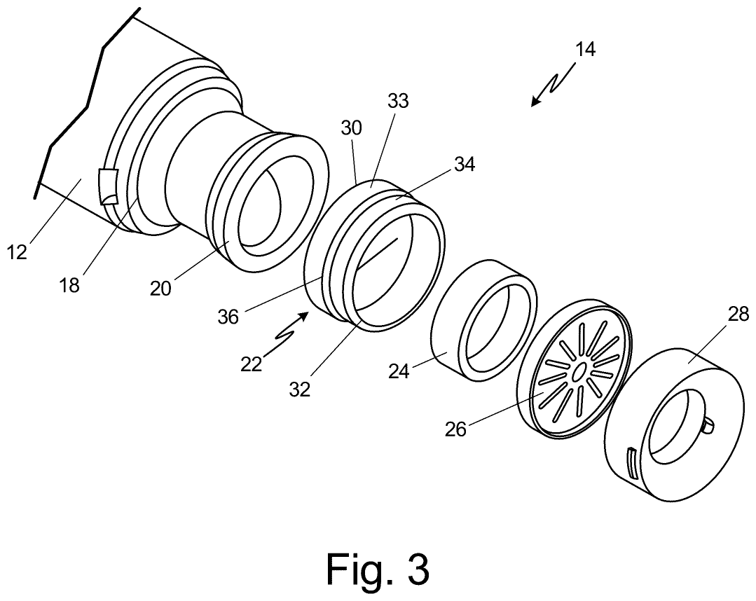

FIG. 2A is a cross-sectional view of seal hub 12 of guide sheath assembly 10 showing seal stack 14 assembled within seal hub 12. FIG. 2B is a disassembled view of seal hub 12 of guide sheath assembly 10 showing seal stack 14 disassembled. FIG. 3 is an exploded perspective view of seal stack 14. FIGS. 2A, 2B, and 3 will be discussed together. Seal hub 12 includes distal end 16 (shown in FIGS. 2A and 2B) and proximal end 18. Seal stack 14 includes duckbill seal 20, cross-plane seal 22, ring 24, disc seal 26, and cap 28. Cross-plane seal 22 includes distal end 30, proximal end 32, disc 33, annular portion 34, and step 36.

Distal end 16 of seal hub 12 is an open first end of seal hub 12. Distal end 16 of seal hub 12 bonds to a sheath shaft of guide sheath assembly 10. Proximal end 18 of seal hub 12 is an open second end of seal hub 12 opposite distal end 16. A medical device, such as a guidewire, a loader, or any other suitable device, can be extended through seal hub 12 from open proximal end 18 to open distal end 16 and retracted through seal hub 12 from distal end 16 through proximal end 18. When a medical device is seated within seal hub 12 of guide sheath assembly 10, the medical device extends past distal end 16 and proximal end 18 of seal hub 12. Seal hub 12 houses seal stack 14.

Duckbill seal 20 is at a distal end of seal stack 14. As such, duckbill seal 20 is the most distal in seal stack 14 and is inserted into seal hub 12 first when assembling seal stack 14. Duckbill seal 20 is a one-way valve that seals better in high pressure systems, such as atrial pressures, and can also be used in low pressure systems, such as venous pressures. In other examples, seal stack 14 does not include duckbill seal 20. In an example not including duckbill seal 20, seal stack 14 may include a cross-slit seal in place of duckbill seal 20. Cross-plane seal 22 is inserted into seal hub 12 second when assembling seal stack 14, after duckbill seal 20, such that cross-plane seal 22 is adjacent duckbill seal 20. Cross-plane seal 22 is made of material having high elongation, high tear resistance, and a low durometer. Ring 24 is inserted into seal hub 12 third when assembling seal stack 14, after cross-plane seal 22. Ring 24 is sized to fit within cross-plane seal 22. Ring 24 is housed, or positioned, within an inner diameter of an annular portion of cross-plane seal 22. Ring 24 and seal hub 12 can be made of the same material. Disc seal 26 is inserted into seal hub 12 fourth when assembling seal stack 14, after ring 24. As such, disc seal 26 is adjacent cross-plane seal 22 and ring 24. Disc seal 26 has a central orifice and raised edges to minimize frictional forces. Disc seal 26 is thin and made of material having a low durometer for easier device insertion. Disc seal 26 is the most proximal seal within seal stack 14. In one example, seal stack 14 does not include duckbill seal 20 or disc seal 26 and has a cross-slit seal in place of disc seal 26. Cap 28 is inserted into seal hub 12 fifth when assembling seal stack 14, after disc seal 26. As such, cap 28 is adjacent disc seal 26. Cap 28 partially fits within seal hub 12 and is only partially inserted into seal hub 12. A distal end of cap 28 interfaces with and mates against disc seal 26, the most proximal seal of seal stack 14. Cap 28 acts as a cover for a proximal end of seal hub 12. Cap 28 has wings that snap fit within seal hub 12, locking cap 28 in place. By locking cap 28 in place, duckbill seal 20, cross-plane seal 22, ring 24, and disc seal 26, which fit within seal hub 12, are also locked into place within seal hub 12.

Cross-plane seal 22 has distal end 30, which is a closed first end of cross-plane seal 22. Distal end 30 contacts duckbill seal 20. Proximal end 32 is an open second end of cross-plane seal 22 opposite distal end 30. Ring 24 is adjacent proximal end of cross-plane seal 22. Cross-plane seal 22 has a body that includes disc 33 and annular portion 34. Disc 33 is at distal end 30 of cross-plane seal 22. Disc 33 contacts duckbill seal 20. Disc 33 is cylindrical, having a thickness. For example, disc 33 can have a thickness of ⅛ inch (0.3175 centimeter). Disc 33 has a greater outer diameter than the outer diameter of annular portion 34. Annular portion 34 is connected to and extends proximally away from disc 33 to proximal end 32. As such, the cross-sectional profile of cross-plane seal 22 is U-shaped. Ring 24 is housed within annular portion 34. Step 36 is formed by disc 33 having a greater outer diameter than the outer diameter of annular portion 34. Step 36 is between disc 33 and annular portion 34. As such, step 36 is adjacent disc 33 of cross-plane seal 22.

A medical device can be inserted through seal hub 12 of guide sheath assembly 10 and into the body of a patient. Seal hub 12 interacts with seal stack 14, which provides a seal for guide sheath assembly 10. Duckbill seal 20 of seal stack 14 acts as a one-way valve to provide a seal when nothing is present, or inserted into, in guide sheath assembly 10. As a result, duckbill seal 20 prevents hemostasis loss when no medical device is inserted through guide sheath assembly 10. As the most distal seal in seal stack 14, duckbill seal 20 also provides a baseline amount of sealing when a medical device is present in guide sheath assembly 10.

Cross-plane seal 22 seals on the medical device present in guide sheath assembly 10, such as the guidewire, the loader, or any other suitable device. The outer diameter of disc 33 of cross-plane seal 22 radially seals against seal hub 12. The outer diameter of disc 33 forms the sealing surface that has an interference fit within seal hub 12. As such, the outer diameter of disc 33 abuts and seals against the inner diameter of seal hub 12, providing a seal between seal hub 12 and seal stack 14. The outer diameter of disc 33 of cross-plane seal 22 also interacts with seal hub 12 to provide retention within seal hub 12. Cross-plane seal 22 is made of a flexible material that lowers insertion and retraction pressure. When a medical device, such as a loader with a large outer diameter, is inserted into cross-plane seal 22, annular portion 34 of cross-plane seal 22 flexes and expands radially. Duckbill seal 20 anchors cross-plane seal 22, and disc seal 26 allows space for the distortion of cross-plane seal 22. Space within seal hub 12 is also available for annular portion 34 of cross-plane seal 22 to expand radially due to the smaller outer diameter of annular portion 34. As cross-plane seal 22 distorts, the counterforce of the insertion is minimized, helping to retain seal stack 14 within seal hub 12.

Ring 24 acts as a spacer and supporting feature within annular portion 34 of cross-plane seal 22. Ring 24 assists in maintaining retention of cross-plane seal 22 against seal hub 12. Ring 24 keeps cross-plane seal 22 spaced from disc seal 26, anchoring disc seal 26 to aid in retention during insertion and retraction of the medical device through guide sheath assembly 10. Ring 24 prevents cross-plane seal 22 from caving or folding in on itself. Disc seal 26 optimizes the insertion of a device through guide sheath assembly 10. Disc seal 26 seals around the inserted medical device, providing redundancy with cross-plane seal 22. Cap 28 closes off seal hub 12 and presses against disc seal 26 to compress seal stack 14 such that seal stack 14 seals against the inner diameter of seal hub 12. Cap 28 locks into place to retain seal stack 14 in seal hub 12 (e.g. during insertion and retraction of the medical device).

Cross-plane seal 22 provides a seal when nothing is inserted in guide sheath assembly 10, having redundancy with duckbill seal 20 for low pressure venous systems, and when devices of various diameters (ranging from about 0.89 millimeter to about 8.13 millimeters) are inserted through guide sheath assembly 10. As such, hemostasis loss is prevented during all portions of the medical procedure, including when guide sheath assembly 10 is first inserted into the patient, when a medical device is inserted into guide sheath assembly 10, and when a medical device is withdrawn from guide sheath assembly 10. Insertion and retraction of a medical device through cross-plane seal 22 is easier, and cross-plane seal 22 can withstand the insertion and retraction of larger devices. Hemostasis loss from cross-plane seal 22 caving or folding in on itself is reduced. Guide sheath assembly 10 is more successful at mitigating hemostasis loss for the patient and can be used in conjunction with a broader variety of medical devices and procedural configurations.

Duckbill seal 20 provides a baseline amount of sealing when a medical device is present in guide sheath assembly 10 to provide redundancy within seal stack 14 and decrease the amount of sealing required by cross-plane seal 22 and disc seal 26. Disc seal 26 also provides redundancy with cross-plane seal 22 to prevent hemostasis loss when a medical device is inserted within seal stack 14.

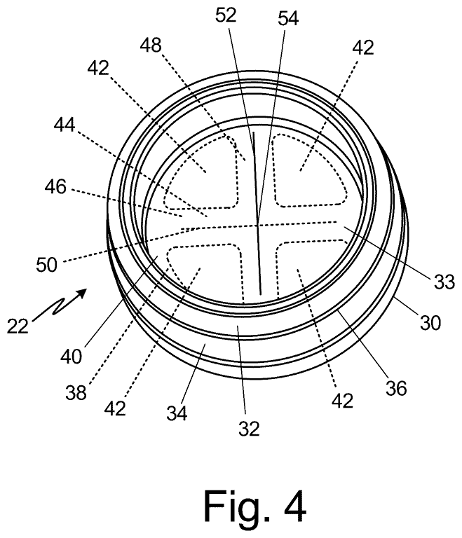

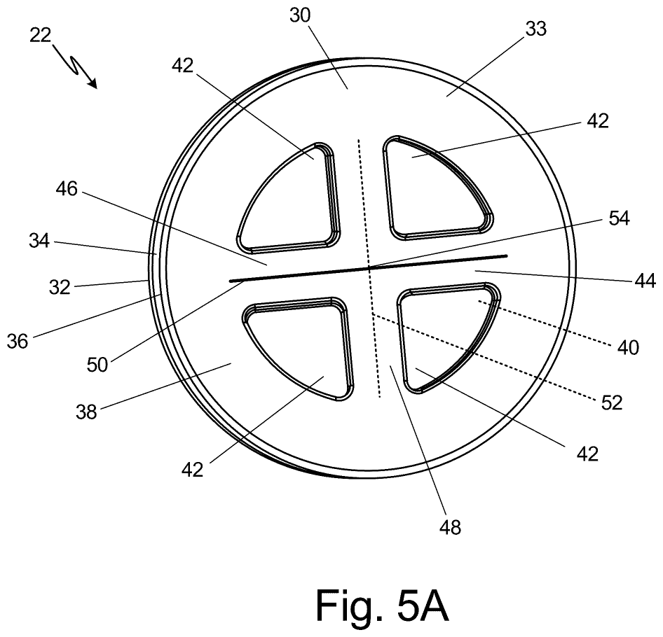

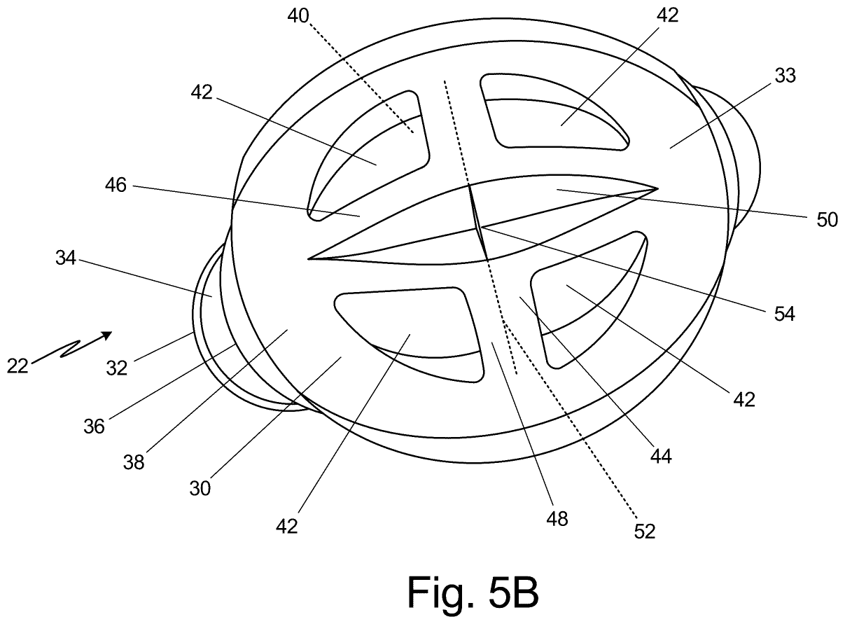

FIG. 4 is a perspective view of cross-plane seal 22 from proximal end 32 of cross-plane seal 22. FIG. 5A is a perspective view of cross-plane seal 22 from distal end 30 of cross-plane seal 22. FIG. 5B is a perspective view of cross-plane seal 22 from distal end 30 of cross-plane seal 22 showing intersection 54 between partial through-cut first slit 50 and partial through-cut second slit 52 when cross-plane seal 22 is deformed. FIGS. 4, 5A, and 5B will be discussed together. Cross-plane seal 22 includes distal end 30, proximal end 32, disc 33, annular portion 34, and step 36. Disc 33 includes distal face 38, proximal face 40, cut outs 42, raised X-shape 44, which includes first leg 46 and second leg 48, first slit 50, second slit 52, and intersection 54.

Cross-plane seal 22 is made of a biocompatible polymer having high elongation, high tear resistance, and a low durometer. Cross-plane seal 22 has a high elongation percentage, preferably 900 percent to 1500 percent. For example, cross-plane seal 22 may have an elongation percentage of about 1490 percent. The elongation percentage of cross-plane seal 22 is selected based on the needs of the application of cross-plane seal 22, such as the size of the medical device that will be inserted through cross-plane seal 22. Cross-plane seal 22 preferably has a durometer of about 20 A (e.g. HCR silicone material). Cross-plane seal 22 may also have any other suitable durometer, including a durometer higher than 20 A or lower than 20 A. Cross-plane seal 22 has a U-shaped cross-sectional profile with a closed distal end 30 and an open proximal end 32. Disc 33 is at distal end 30 of cross-plane seal 22. As such, disc 33 of cross-plane seal 22 is between duckbill seal 20 and ring 24. Disc 33 can have a thickness of ⅛ inch (0.3175 centimeter). Disc 33 has a greater outer diameter than the outer diameter of annular portion 34. Annular portion 34 extends from disc 33 to proximal end 32. Step 36 is formed where disc 33 is connected to annular portion 34.

Disc 33 has distal face 38 at the distal side of disc 33 and proximal face 40 at the proximal side of disc 33. Distal face 38 is at distal end 30 of cross-plane seal 22. Disc 33 has a thickness between distal face 38 and proximal face 40. Distal face 38 has a plurality of cut outs 42. In this example, distal face 38 has four cut outs. Cut outs 42 are recesses in distal face 38 of disc 33, each cut out 42 is wedge-shaped, having two lines that are perpendicular to one another and a curved third line connecting the first two lines (each connection between the lines being curved rather than pointed). Cut outs 42 are positioned within distal face 38 of disc 33 to form raised X-shape 44 on distal face 38 of disc 33 at distal end 30 of cross-plane seal 22. As such, raised X-shape 44 is pressed against a raised edge of a proximal end of duckbill seal 20 (shown in FIGS. 2 and 3) when cross-plane seal 22 is within seal stack 14. Raised X-shape 44 comprises first leg 46 and second leg 48. First leg 46 of raised X-shape 44 is on distal face 38 of disc 33. Second leg 48 of raised X-shape 44 is on distal face 38 of disc 33 and is perpendicular to first leg 46.

First slit 50 is a slit having a partial through-cut into distal face 38 of disc 33. First slit 50 is aligned with first leg 46 of raised X-shape 44. The through-cut of first slit 50 extends more than halfway, but not fully, through the thickness of disc 33. For example, first slit 50 may extend 3/32 inch (2.38 millimeters) deep through disc 33 having a material thickness of ⅛ inch (3.175 millimeters). In this example, first slit 50 may be ½ inch (12.7 millimeters) long within disc 33. First slit 50 can extend through between 50 percent and 100 percent of the thickness of disc 33. Second slit 52 is a slit having a partial through-cut into proximal face 40 of disc 33. Second slit 52 is aligned with second leg 48 of raised X-shape 44. The through-cut of second slit 52 extends more than halfway, but not fully, through the thickness of disc 33. For example, second slit 52 may extend 3/32 inch (2.38 millimeters) deep through disc 33 having a material thickness of ⅛ inch (3.175 millimeters). In this example, second slit 52 may be ½ inch (12.7 millimeters) long within disc 33. Second slit 52 can extend through between 50 percent and 100 percent of the thickness of disc 33. First slit 50 is perpendicular to second slit 52. A medical grade lubricant, such as Christo-lube or silicone oil, may be on first slit 50 and/or second slit 52. Because first slit 50 extends more than halfway through disc 33 from distal face 38, second slit 52 extends more than halfway through disc 33 from proximal face 40, and first slit 50 and second slit 52 are perpendicular, first slit 50 and second slit 52 partially overlap to form intersection 54. Intersection 54 is within disc 33 at a center of raised X-shape 44. Intersection 54 forms an opening, or pathway, through disc 33 between proximal face 40 of disc 33 and distal face 38 of disc 33.

As a medical device is inserted through cross-plane seal 22, the medical device exerts force on proximal face 40 of disc 33 of cross-plane seal 22. Intersection 54 of first slit 50 and second slit 52 is forced open as the material of cross-plane seal 22 elongates in response to the insertion force. Cut outs 42 on disc 33 of cross-plane seal 22 provide material relief upon the insertion of the medical device and minimize frictional force interaction with the medical device. As such, cut outs 42 cause disc 33 to open more easily, and thus, allow for accommodation of a larger diameter medical device. For example, cut outs 42 allow for a loader tube with a larger outer diameter and a larger introduction force on disc 33 to be inserted into cross-plane seal 22. Intersection 54 is a dynamic region within cross-plane seal 22. As intersection 54 is forced open, an orifice is formed at intersection 54.

The medical device is extended through the orifice at intersection 54. When the medical device is positioned within cross-plane seal 22, first slit 50 and second slit 52 seal around the medical device. The material of cross-plane seal 22 improves the seal on the medical device. Additionally, first slit 50 and second slit 52 are positioned perpendicular to each other and on opposite faces of disc 33 to provide a constant sealing effect, regardless of whether the medical device is extending through cross-plane seal 22 coaxially. As such, the medical device may be coaxial or non-coaxial to cross-plane seal 22. Further, first slit 50 and second slit 52 each extend less than fully through disc 33, or are partial through-cut slits, to provide a sealing region that is also able to retain the medical device that is inserted coaxially or non-coaxially. Cut outs 42 assist in retention as well. Because cut outs 42 are only on distal face 38 of disc 33, cross-plane seal 22 has a greater ability to retain a medical device within cross-plane seal 22. The retention ability of cross-plane seal 22 is especially important for a medical device that is inserted or retracted non-coaxially.

The medical device can be retracted through the orifice at intersection 54 to remove the medical device from cross-plane seal 22. When the medical device is retracted, the material of cross-plane seal 22 lowers the force caused by retraction. First slit 50 and second slit 52 return to their original positions, and the orifice at intersection 54 of disc 33 disappears. Because first slit 50 and second slit 52 only partially overlap, cross-plane seal 22 provides an effective seal when no device is present within cross-plane seal 22. Additionally, cross-plane seal 22 is less likely to cave or fold in on itself, easing insertion and retraction of medical devices into and from cross-plane seal 22.

Limited space is available in catheter labs in the clinical environment. As a result, it may not be possible to orient a medical device through guide sheath assembly 10 coaxially, or straight from the access site of guide sheath assembly 10. For example, a medical device may be tracked over a guidewire into guide sheath assembly 10 and oriented perpendicularly to the incision site such that the medical device enters the incision along a curved path. Thus, the medical device enters guide sheath assembly 10, and cross-plane seal 22, along a curved path. A curved path presents a greater risk of a fluid leak, which may lead to hemostasis loss.

Cross-plane seal 22 reduces the risk of hemostasis loss. Because cross-plane seal 22 of seal stack 14 provides improved sealing upon insertion and retraction of a medical device both coaxially and non-coaxially, cross-plane seal 22 reduces the risk of fluid leakage. Cross-plane seal 22 can also accommodate a larger array of medical devices while preventing hemostasis loss. Further, cross-plane seal 22 allows for easier device insertion, retention, and retraction within guide sheath assembly 10.

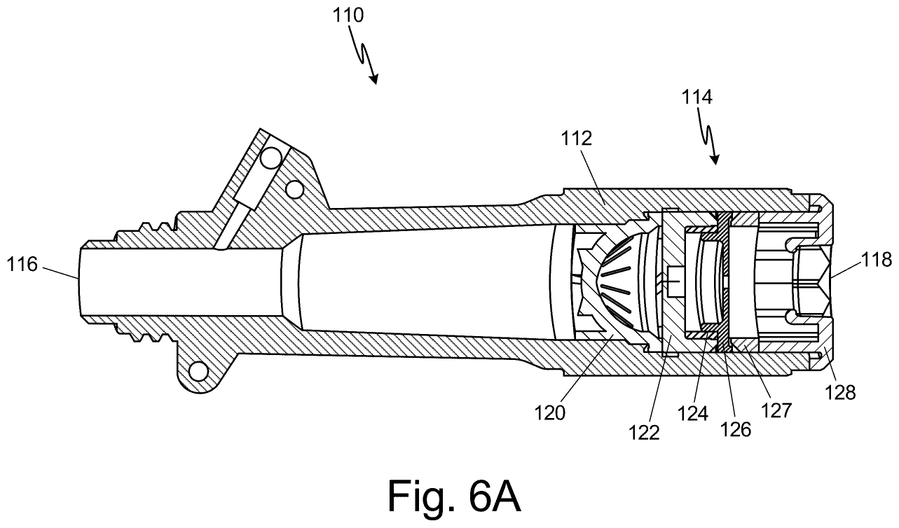

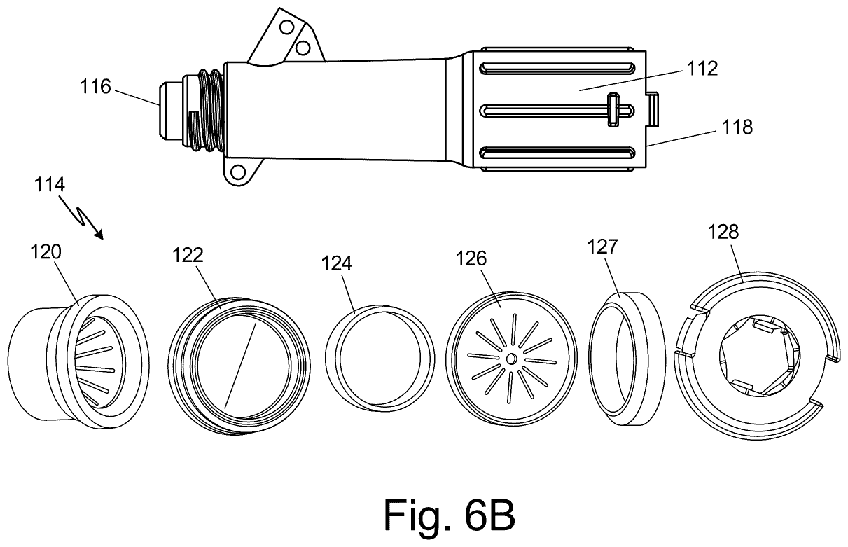

FIG. 6A is a cross-sectional view of seal hub 112 of guide sheath assembly 110 showing seal stack 114 assembled within seal hub 112. FIG. 6B is a disassembled view of seal hub 112 of guide sheath assembly 110 showing seal stack 114 disassembled. FIGS. 6A and 6B will be discussed together. Seal hub 112 includes distal end 116 and proximal end 118. Seal stack 114 includes cross-slit seal 120, cross-plane seal 122, ring 124, disc seal 126, spacer 127, and cap 128.

Guide sheath assembly 110 has the same structure and function as guide sheath assembly 10 described with respect to FIGS. 1-5B. However, guide sheath assembly 110 has seal stack 114 that includes cross-slit seal 120 rather than duckbill seal 20 and has spacer 127. Seal hub 112 has a slightly different structure than seal hub 12 while still providing the same function.

Cross-slit seal 120 is at a distal end of seal stack 114. As such, cross-slit seal 120 is the most distal in seal stack 114 and is inserted into seal hub 112 first when assembling seal stack 114. Cross-plane seal 122 is adjacent cross-slit seal 120 such that cross-plane seal 122 is proximal to cross-slit seal 120 when assembling seal stack 114. Cross-slit seal 120 seals on a medical device inserted through seal hub 112. Cross-slit seal 120 provides a baseline amount of sealing when a medical device is present in guide sheath assembly 110 to provide redundancy within seal stack 114 and decreases the amount of sealing required by cross-plane seal 122 and disc seal 126. Spacer 127 is positioned between disc seal 126 and cap 128 in seal stack 114. Spacer 127 is proximal to disc seal 126 and distal to cap 128 within seal stack 114. Spacer 127 is inserted into seal hub 112 fifth when assembling seal stack 114. Spacer 127 provides space between flexible disc seal 126 and cap 128 during retraction of a medical device through seal hub 112. Seal stack 114 including cross-slit seal 120 reduces the risk of hemostasis loss when using guide sheath assembly 110.

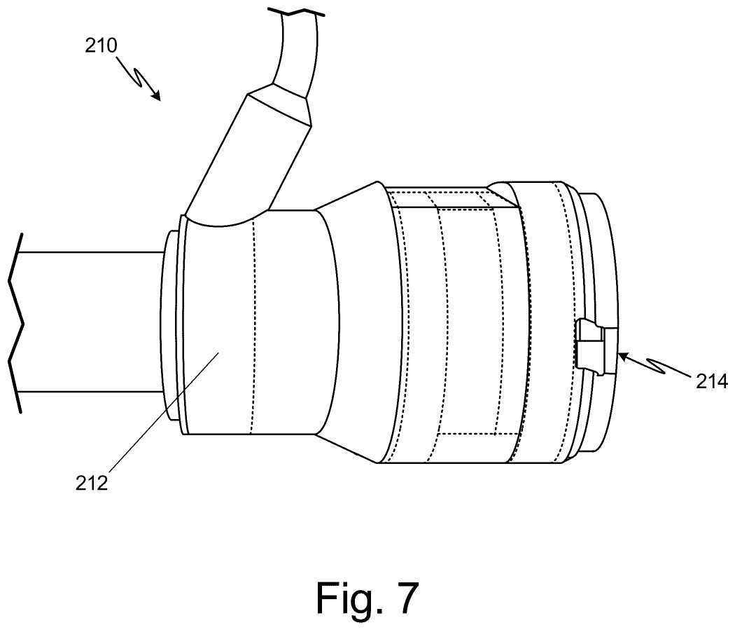

FIG. 7 is a side view of seal hub 212 of guide sheath assembly 210 showing seal stack 214 within seal hub 212. Guide sheath assembly 210 includes seal hub 212 and seal stack 214.

Guide sheath assembly 210 is configured to introduce a medical device into the body of a patient. A distal end of guide sheath assembly 210 is configured to be inserted into the body of the patient. A proximal end of guide sheath assembly 210 is configured to be outside of the body of the patient and is accessible by a healthcare worker. Seal hub 212 is at the proximal end of guide sheath assembly 210. Seal hub 212 may be housed within a handle, which may also be configured to flush and aspirate. Seal hub 212 accepts insertion of a medical device through seal stack 214 from a proximal end of seal hub 212. Seal hub 212 of guide sheath assembly 210 provides a housing for seal stack 214. As such, seal stack 214 fits within seal hub 212 of guide sheath assembly 210. Seal stack 214 comprises a plurality of seals, which are assembled within seal hub 212. Seal stack 214 accepts insertion of a medical device through a proximal end of seal stack 214 such that seal stack 214 surrounds a portion of the medical device.

An incision is made into the body of a patient to insert guide sheath assembly 210 into the patient. For example, an incision can be made into the neck of a patient such that guide sheath assembly 210 has jugular access into the body of the patient. A distal portion of guide sheath assembly 210 is inserted into the body of the patient through the incision. A medical device, such as a guidewire, a loader, an introducer, or any other suitable device, can be inserted into the body of the patient through guide sheath assembly 210. As such, guide sheath assembly 210 provides a port into the body of the patient. To maintain hemostasis by preventing blood loss through guide sheath assembly 210, seal stack 214 is included in seal hub 212 of guide sheath assembly 210. Seal stack 214 can provide a seal when nothing is inserted in guide sheath assembly 210. Seal stack 214 can also provide a seal when a medical device, such as the guidewire, the loader, the introducer, or any other device, is inserted into guide sheath assembly 210. Seal stack 214 seals on the guidewire, the loader, or any other medical device inserted into guide sheath assembly 210 to prevent fluid loss, such as blood loss. Seal stack 214 seals around the inserted medical device and retains the position of the medical device within seal stack 214.

Seal stack 214 in seal hub 212 of guide sheath assembly 210 helps prevent blood loss from the patient while guide sheath assembly 210 is inserted into the patient. Seal stack 214 in seal hub 212 provides a seal when nothing is present in guide sheath assembly 210 and when a medical device is inserted into guide sheath assembly 210. As such, seal stack 214 prevents hemostasis loss, which reduces the risk of cardiogenic shock caused by a sustained loss of hemostasis. As a result, the amount of blood transfusions needed for the patient is reduced and healthcare worker satisfaction is increased.

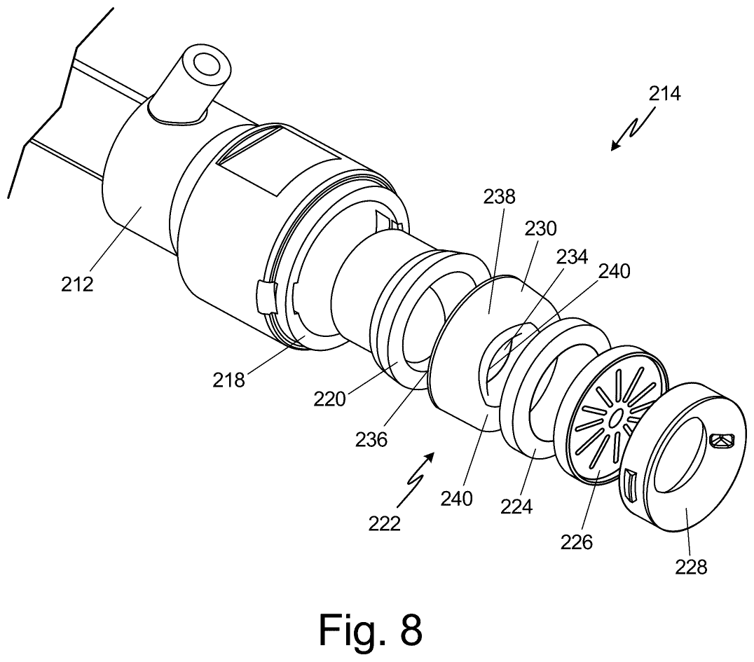

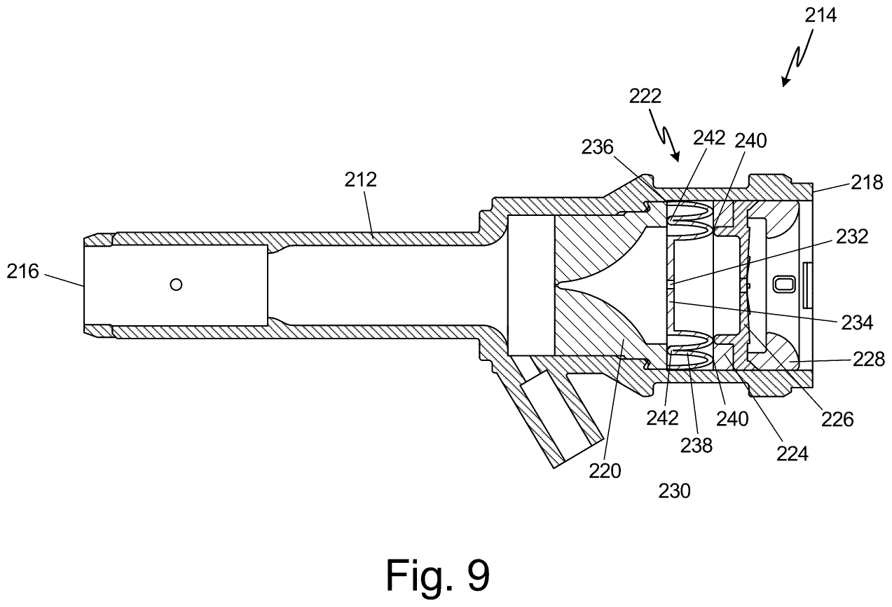

FIG. 8 is an exploded view of seal hub 212 of guide sheath assembly 210 showing seal stack 214 with self-centering seal 222. FIG. 9 is a cross-sectional view of seal hub 212 of guide sheath assembly 210 showing seal stack 214 assembled within seal hub 212. FIGS. 8 and 9 will be discussed together. Seal hub 212 includes distal end 216 (shown in FIG. 9) and proximal end 218. Seal stack 214 includes duckbill seal 220, self-centering seal 222, ring 224 (shown in FIG. 8), disc seal 226, and cap 228. Self-centering seal 222 includes disc 230, hole 232 (shown in FIG. 9), and central planar region 234. Disc 230 includes rigid portion 236 (shown in FIG. 8) and plurality of raised curves 238. Plurality of raised curves 238 include hills 240 and valleys 242.

Distal end 216 of seal hub 212 is an open first end of seal hub 212. Distal end 216 of seal hub 212 bonds to a sheath shaft of guide sheath assembly 210. Proximal end 218 of seal hub 212 is an open second end of seal hub 212 opposite distal end 216. A medical device, such as a guidewire, a loader, or any other suitable device, can be extended through seal hub 212 from open proximal end 218 to open distal end 216 and retracted through seal hub 212 from distal end 216 through proximal end 218. When a medical device is seated within seal hub 212 of guide sheath assembly 210, the medical device extends past distal end 216 and proximal end 218 of seal hub 212. Seal hub 212 houses seal stack 214.

Duckbill seal 220 is between distal end 216 and proximal end 218 of seal hub 212. Duckbill seal 220 is at a distal end of seal stack 214. As such, duckbill seal 220 is the most distal seal in seal stack 214 and is inserted into seal hub 212 first when assembling seal stack 214. Duckbill seal 220 is a one-way valve that seals better in high pressure systems, such as atrial pressures, and can also be used in low pressure systems, such as venous pressures. In other examples, seal stack 214 does not include duckbill seal 220. In an example not including duckbill seal 220, seal stack 214 includes self-centering seal 222 in place of duckbill seal 220.

Self-centering seal 222 is inserted into seal hub 212 second when assembling seal stack 214, after duckbill seal 220. As such, self-centering seal 222 is adjacent to duckbill seal 220. Self-centering seal 222 is radially symmetric and disc-shaped. Self-centering seal 222 is made of material with high elongation, high tear resistance, and low durometer.

Ring 224 is inserted into seal hub 212 third when assembling seal stack 214, after self-centering seal 222. As such, ring 224 is adjacent to self-centering seal 222. Ring 224 and seal hub 212 can be made from the same material. Disc seal 226 is inserted into seal hub 212 fourth when assembling seal stack 214, after ring 224. As such, disc seal 226 is adjacent to self-centering seal 222 and ring 224.

Disc seal 226 has a central orifice and raised edges to minimize frictional forces. Disc seal 226 is thin and made of material having a low durometer for easier device insertion. Disc seal 226 is the most proximal seal within seal stack 214. In one example, seal stack 214 does not include duckbill seal 220 or disc seal 226 and has self-centering seal 222 in place of disc seal 226.

Cap 228 is inserted into seal hub 212 fifth when assembling seal stack 214, after disc seal 226. As such, cap 228 is adjacent to disc seal 226. Cap 228 partially fits within seal hub 212 and is only partially inserted into seal hub 212. A distal end of cap 228 interfaces with and mates against disc seal 226, the most proximal seal of seal stack 214. Cap 228 acts as a cover for proximal end 218 of seal hub 212. Cap 228 has wings that snap fit within seal hub 212, locking cap 228 in place. By locking cap 228 in place, duckbill seal 220, self-centering seal 222, ring 224, and disc seal 226, which fit within seal hub 212, are also locked into place within seal hub 212.

Disc 230 is an annular outer part of self-centering seal 222. An outer diameter of disc 230 of self-centering seal 222 has an interference fit within seal hub 212 and forms a sealing surface with seal hub 212. Hole 232 is located inside disc 230, within central planar region 234. Hole 232 is a space that extends through a center of flat central planar region 234. Hole 232 is sized and shaped to accept insertion of a medical device. Disc 230 is connected to and surrounds central planar region 234. An inner portion, or inner diameter, of disc 230 is connected to an outer portion of central planar region 234.

Rigid portion 236 of disc 230 makes up an outer edge of disc 230. Rigid portion 226 may be made of a different material than the rest of disc 230. Plurality of raised curves 238 of disc 230 extend axially from central planar region 234. Plurality of raised curves 238 form two U-shaped concentric rings that are connected to each other. Plurality of raised curves 238 comprise hills 240 and valleys 242. Hills 240 are convex portions of plurality of curves 238, and valleys 242 are concave portions of plurality of curves 238. As such, hills 240 and valleys 242 form plurality of curves 238. A distal end of disc 230 has one hill 240 and two valleys 242. A proximal end of disc 230 has two hills 240 and one valley 242.

A medical device can be inserted through seal hub 212 of guide sheath assembly 210 and into the body of a patient. Seal hub 212 interacts with seal stack 214, which provides a seal for guide sheath assembly 210. A distal end of guide sheath assembly 210 is configured to be inserted into the body of the patient. A proximal end of guide sheath assembly is configured to be outside of the body of the patient and is accessible by a healthcare worker. Seal hub 212 may be housed within a handle, which may also be configured to flush and aspirate. Seal hub 212 accepts insertion of a medical device through proximal end 218 of seal hub 212 and seal stack 214. Seal hub 212 of guide sheath assembly 210 provides a housing for seal stack 214. As such, seal stack 214 fits within seal hub 212 of guide sheath assembly 210. Seal stack 214 comprises a plurality of seals, which are assembled within seal hub 212. Seal stack 214 accepts insertion of a medical device through a proximal end of seal hub 212 such that seal stack 214 surrounds a portion of the medical device.

Duckbill seal 220 of seal stack 214 acts as a one-way valve to provide a seal when nothing is present in, or inserted into, guide sheath assembly 210. As a result, duckbill seal 220 prevents hemostasis loss when no medical device is inserted through guide sheath assembly 210. As the most distal seal in seal stack 214, duckbill seal 220 also provides a baseline amount of sealing when a medical device is present in guide sheath assembly 210.

Self-centering seal 222 of seal stack 214 acts to seal when a medical device is inserted into seal hub 212. The medical device is inserted through hole 232. The medical device may be inserted off-axis on a plane not substantially parallel to seal hub 212. Plurality of raised curves 238 of self-centering seal 222 narrow and expand on different sides to shift hole 232 and central planar region 234 off-axis of seal stack 214, preventing leaks. Self-centering seal is made from a biocompatible polymer. The biocompatible polymer can have a durometer of about 40 A and an elongation percentage of 500 percent to 1200 percent.

Ring 224 acts as a spacer and supporting feature of self-centering seal 222. Ring 224 assists in maintaining retention of self-centering seal 222 against duckbill seal 220. Ring 224 keeps self-centering seal 222 adjacent to duckbill seal 220 and spaced from disc seal 226, anchoring disc seal 226 to aid in retention during insertion and retraction of the medical device through guide sheath assembly 210. In other embodiments, self-centering seal 222 may be touching disc seal 226 (see FIG. 9). Disc seal 226 optimizes the insertion of a device through guide sheath assembly 210. Disc seal 226 seals around the inserted medical device, providing redundancy with self-centering seal 222. Cap 228 closes off seal hub 212 and presses against disc seal 226 to compress seal stack 214 such that seal stack 214 seals against the inner diameter of seal hub 212. Cap 228 locks into place to retain seal stack 214 in seal hub 212 (e.g. during insertion and retraction of the medical device).

Self-centering seal 222 provides a seal when nothing is inserted in guide sheath assembly 210, having redundancy with duckbill seal 220 for low pressure venous systems, and when devices of various diameters (ranging from about 0.89 millimeter to about 8.13 millimeters) are inserted through guide sheath assembly 210. Self-centering seal 222 also provides superior leak prevention when medical device insertion is made off-axis and not directly on-plane with seal stack 214 or substantially parallel to seal hub 212. Plurality of raised curves 238 allow for additional side-to-side motion during insertion by flexing and allowing for additional movement of an inserted device without compromising sealing. As such, hemostasis loss is prevented during all portions of the medical procedure, including when guide sheath assembly 210 is first inserted into the patient, when a medical device is inserted into guide sheath assembly 210, and when a medical device is withdrawn from guide sheath assembly 210. Insertion and retraction of a medical device through self-centering seal 222 is easier and does not have to be as precise. Self-centering seal 222 can withstand the insertion and retraction of larger devices. Guide sheath assembly 210 is more successful at mitigating hemostasis loss for the patient and can be used in conjunction with a broader variety of medical devices and procedural configurations.

Duckbill seal 220 provides a baseline amount of sealing when a medical device is present in guide sheath assembly 210 to provide redundancy within seal stack 214 and decrease the amount of sealing required by self-centering seal 222 and disc seal 226. Disc seal 226 also provides redundancy with self-centering seal 222 to prevent hemostasis loss when a medical device is inserted within seal stack 214.

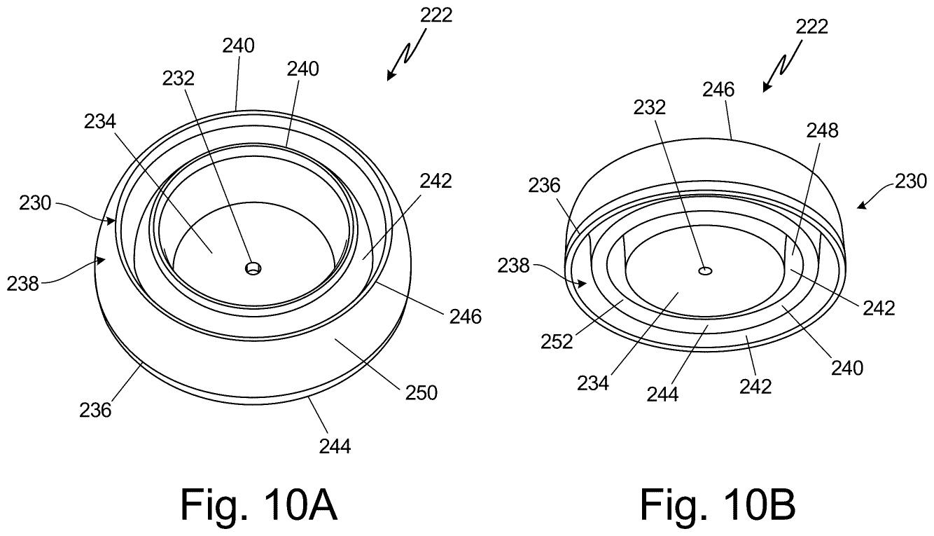

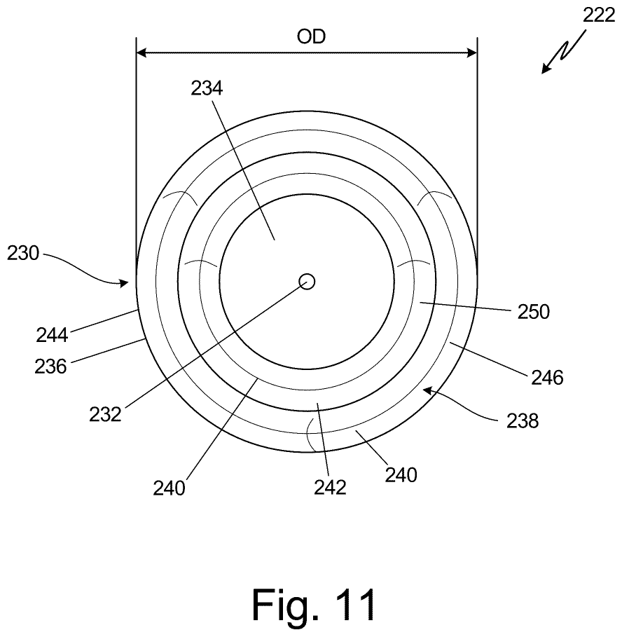

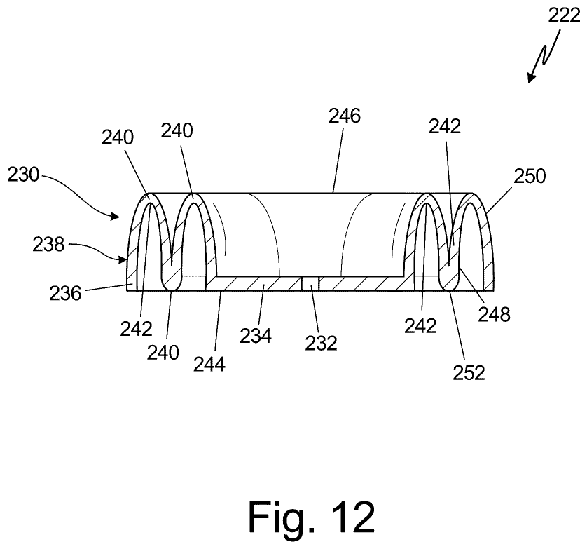

FIG. 10A is a perspective view of self-centering seal 222 from proximal end 246 of self-centering seal 222. FIG. 10B is a perspective view of self-centering seal 222 from distal end 246 of self-centering seal 222. FIG. 11 is a front plan view of self-centering seal 222. FIG. 12 is a cross-sectional side view of self-centering seal 222. FIGS. 10A, 10B, 11, and 12 will be discussed together.

Self-centering seal 222 includes disc 230, hole 232, and central planar region 234. Disc 230 includes rigid portion 236, plurality of raised curves 238, distal end 244, proximal end 246, distal face 248 (shown in FIGS. 10B and 12), and proximal face 250 (shown in FIGS. 10A, 11, and 12). Plurality of raised curves 238 include hills 240, valleys 242, and flattened region 252 (shown in FIGS. 10B and 12).

Distal end 244 of disc 230 is at a first end of disc 230, and proximal end 246 of disc 230 is at a second end of disc 230 opposite first end of disc 230. Distal end 244 of disc 230 makes up a distal end of self-centering seal 222. Proximal end 246 of disc 230 makes up a proximal end of self-centering seal 222. Rigid portion 236 of disc 230 is at distal end 244 of disc 230. Plurality of raised curves 238 extend proximally from distal end 244 of disc 230 to proximal end 246 of disc 230, along seal stack 214. Plurality of raised curves 238 have a length between 300 percent and 1000 percent the thickness of self-centering seal 222. In alternate embodiments, plurality of raised curves 238 may have different lengths. Distal face 248 of disc 230 is a surface of disc 230 extending from distal end 244 of disc 230, and proximal face 250 is a surface of disc 230 extending from proximal end 246 of disc 230. One hill 240 and two valleys 242 of plurality of raised curves 238 at distal end 244 of disc 230 form curves in distal face 248. Two hills 240 and one valley 242 of plurality of raised curves 238 at proximal end 246 of disc 230 form curves in proximal face 250. Hills 240 and valleys 242 are connected to each other. Flattened region 252 of plurality of raised curves 238 is between valleys 242 on distal face 248 of disc 230. As such, flattened region 252 makes up a top portion of hill 240 of plurality of raised curves 238 at distal end 244 of disc 230. Flattened region 252 is concentric to a distal end of central planar region 234, and a distal end of flattened region 252 is planar with a distal end of central planar region 234. In alternate embodiments, plurality of raised curves 238 may not include flattened region 252.

As a medical device is inserted through self-centering seal 222, the medical device exerts force on hole 232 in central planar region 234 of self-centering seal 222. The material of self-centering seal 222 improves the seal on the medical device by allowing for off-axis insertion without self-centering seal 222 being compromised. For example, the medical device may move hole 232 away from a center of self-centering seal 222 during off-axis insertion. Central planar region 234 moves along with hole 232 and causes plurality of raised curves 238 of disc 230 to move, or change shape. Hills 240 and valleys 242 of plurality of raised curves 238 expand and contract to allow movement of hole 232 and central planar region 234 while the outer diameter O.D. (shown in FIG. 11) of disc 230 remains in place. As such, plurality of raised curves 238 allow for hole 232 to move from a center of disc 230 without breaking the seal between self-centering seal 222 and seal hub 212. As such, the medical device may be coaxial or non-coaxial to self-centering seal 222. As such, self-centering seal 222 allows for a medical device to be inserted or retracted non-coaxially.

Limited space is available in catheter labs in the clinical environment. As a result, it may not be possible to orient a medical device through guide sheath assembly 210 coaxially, or straight from the access site of guide sheath assembly 210. For example, a medical device may be tracked over a guidewire into guide sheath assembly 210 and oriented perpendicularly to the incision site such that the medical device enters the incision along a curved path. Thus, the medical device enters guide sheath assembly 210, and self-centering seal 222, along a curved path. A curved path presents a greater risk of a fluid leak, which may lead to hemostasis loss.

Self-centering seal 222 reduces the risk of hemostasis loss. Because self-centering seal 222 of seal stack 214 provides improved sealing upon insertion and retraction of a medical device both coaxially and non-coaxially, self-centering seal 222 reduces the risk of fluid leakage. Seal-centering seal 222 can also accommodate a larger array of medical devices while preventing hemostasis loss. Further, self-centering seal 222 allows for easier device insertion, retention, and retraction within guide sheath assembly 210.

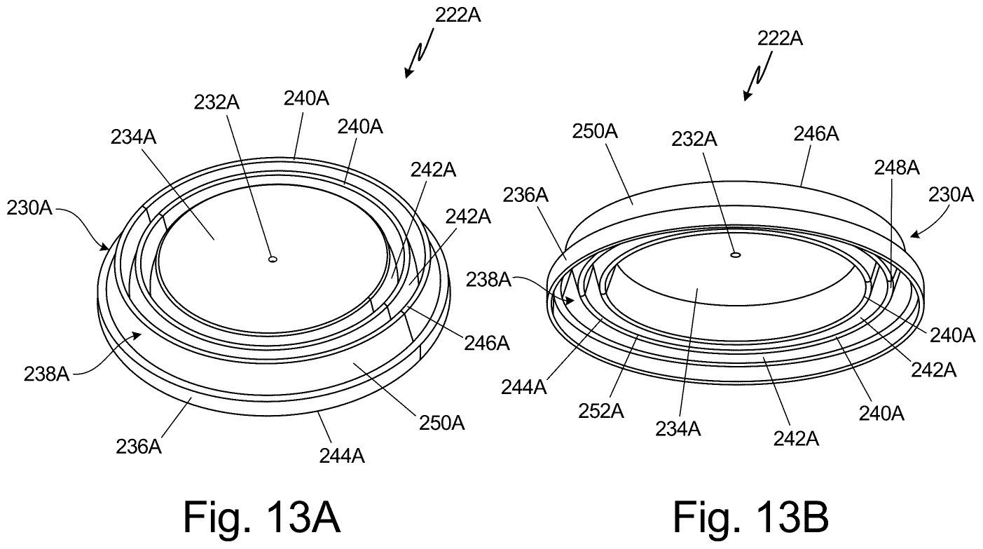

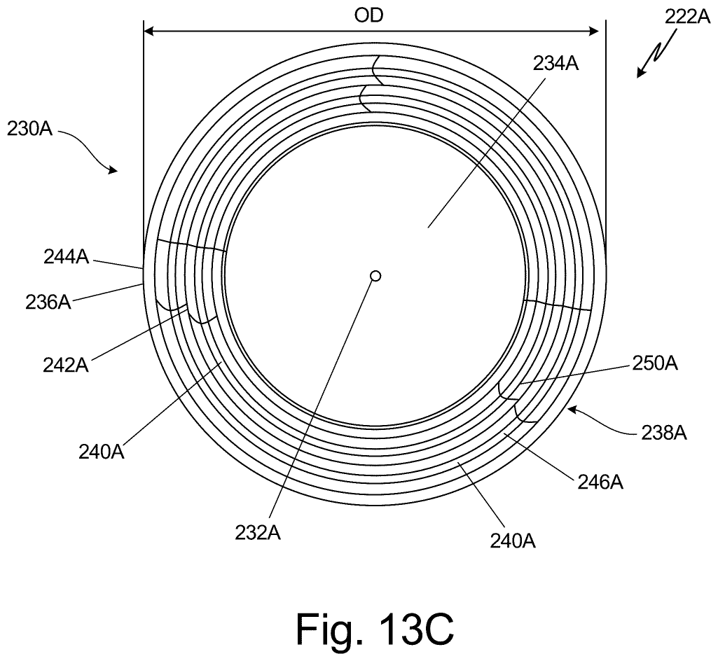

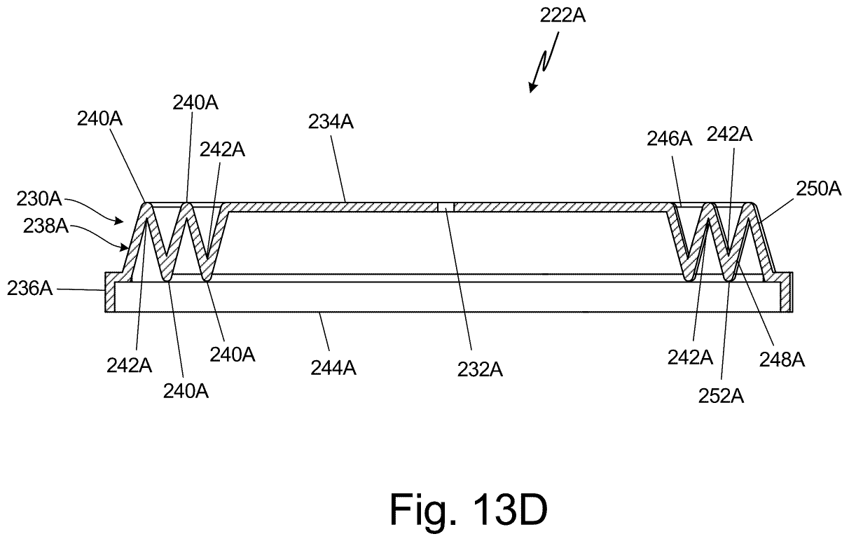

FIG. 13A is a perspective view of self-centering seal 222A from proximal end 246A of self-centering seal 222A. FIG. 13B is a perspective view of self-centering seal 222A from distal end 244A of self-centering seal 222A. FIG. 13C is a front plan view of self-centering seal 222A. FIG. 13D is a cross-sectional side view of self-centering seal 222A. FIGS. 13A, 13B, 13C, 13D will be discussed together.

Self-centering seal 222A includes disc 230A, hole 232A, and central planar region 234A. Disc 230A includes rigid portion 236A, plurality of raised curves 238A (which include hills 240A and valleys 242A), distal end 244A (shown in FIGS. 13B and 13D), proximal end 246A, distal face 248A (shown in FIGS. 13B and 13D), and proximal face 250A. Plurality of raised curves 238A also include flattened region 252A (shown in FIGS. 13B and 13D).

Self-centering seal 222A is radially symmetric and disc-shaped. Self-centering seal 222A is made of material with high elongation, high tear resistance, and low durometer. Self-centering seal is made from a biocompatible polymer, such as polyisoprene. The biocompatible polymer can have a durometer of about 40 A and an elongation percentage of 500 percent to 1200 percent. Disc 230A is an annular outer part of self-centering seal 222A. An outer diameter of disc 230A of self-centering seal 222 has an interference fit within a seal hub, such as seal hub 212 described with respect to FIGS. 7, 8, and 9, and forms a sealing surface with the seal hub. Hole 232A is located inside disc 230, within central planar region 234A. Hole 232A is a space that extends through a center of flat central planar region 234A. Hole 232A is sized and shaped to accept insertion of a medical device. Disc 230A is connected to and surrounds central planar region 234A. An inner portion, or inner diameter, of disc 230A is connected to an outer portion of central planar region 234A.

Rigid portion 236A of disc 230A makes up an outer edge of disc 230A. Rigid portion 326A may be made of a different material than the rest of disc 230A. Plurality of raised curves 238A of disc 230A extend axially from central planar region 234A. Plurality of raised curves 238A form two U-shaped, or V-shaped, concentric rings that are connected to each other. Plurality of raised curves 238A comprise hills 240A and valleys 242A. Hills 240A are convex portions of plurality of curves 238A, and valleys 242A are concave portions of plurality of curves 238A. As such, hills 240A and valleys 242A form plurality of curves 238A. A distal end of disc 230A has two hills 240A and two valleys 242A. A proximal end of disc 230A has two hills 240A and two valley 242A.

Distal end 244A of disc 230A is at a first end of disc 230A, and proximal end 246A of disc 230A is at a second end of disc 230A opposite first end of disc 230A. Distal end 244A of disc 230A makes up a distal end of self-centering seal 222A. Proximal end 246A of disc 230A makes up a proximal end of self-centering seal 222A. Rigid portion 236A of disc 230A is at distal end 244A of disc 230A. Plurality of raised curves 238A extend proximally from distal end 244A of disc 230A to proximal end 246A of disc 230A. Plurality of raised curves 238A have a length greater than the thickness of self-centering seal 222. In alternate embodiments, plurality of raised curves 238A may have different lengths. Distal face 248A of disc 230A is a surface of disc 230A extending from distal end 244A of disc 230A, and proximal face 250A is a surface of disc 230A extending from proximal end 246A of disc 230A. Two hills 240A and two valleys 242A of plurality of raised curves 238A at distal end 244A of disc 230A form curves in distal face 248A. Two hills 240A and two valleys 242A of plurality of raised curves 238A at proximal end 246A of disc 230A form curves in proximal face 250A. A proximal end of central planar region 234A is planar with tops of hills 240A at proximal end 246A of disc 230A. Hills 240A and valleys 242A are connected to each other. Flattened region 252A of plurality of raised curves 238A is between valleys 242A on distal face 248A of disc 230A. As such, flattened region 252A makes up a top portion of hill 240A of plurality of raised curves 238A at distal end 244A of disc 230A. Flattened region 252A is concentric to a distal end of central planar region 234A. In alternate embodiments, plurality of raised curves 238A may not include flattened region 252A.

Self-centering seal 222A can be inserted into a seal hub, such as seal hub 212 described with respect to FIGS. 7, 8, and 9. Self-centering seal 222A can be inserted second when assembling a seal stack, such as seal stack 214 described with respect to FIGS. 8 and 9. As such, self-centering seal 22A may be proximal and adjacent to a duckbill seal, such as duckbill seal 220 described with respect to FIGS. 8 and 9.

Self-centering seal 222A provides a seal when a medical device is inserted into a seal hub. As the medical device is inserted through self-centering seal 222A, the medical device is inserted through hole 232A and exerts force on hole 232A in central planar region 234A of self-centering seal 222A. The material of self-centering seal 222A improves the seal on the medical device by allowing for off-axis insertion without self-centering seal 222A being compromised. For example, the medical device may be inserted off-axis on a plane not substantially parallel to the seal hub. Plurality of raised curves 238A may shift hole 232A and central planar region 234A off-axis to prevent leaks. For example, the medical device may move hole 232A away from a center of self-centering seal 222A during off-axis insertion. Central planar region 234A moves along with hole 232A and causes plurality of raised curves 238A of disc 230A to move, or change shape. Hills 240A and valleys 242A of plurality of raised curves 238A expand and contract to allow movement of hole 232A and central planar region 234A while the outer diameter O.D. (shown in FIG. 13C) of disc 230A remains in place. As such, plurality of raised curves 238A allow for hole 232A to move from a center of disc 230A without breaking the seal between self-centering seal 222A and a seal hub. As such, the medical device may be coaxial or non-coaxial to self-centering seal 222A. As such, self-centering seal 222A allows for a medical device to be inserted or retracted non-coaxially.

Limited space is available in catheter labs in the clinical environment. As a result, it may not be possible to orient a medical device through a guide sheath assembly coaxially, or straight from the access site of the guide sheath assembly. For example, a medical device may be tracked over a guidewire into the guide sheath assembly and oriented perpendicularly to the incision site such that the medical device enters the incision along a curved path. Thus, the medical device enters the guide sheath assembly, and self-centering seal 222A, along a curved path. A curved path presents a greater risk of a fluid leak, which may lead to hemostasis loss.

Self-centering seal 222A reduces the risk of hemostasis loss. Because self-centering seal 222A provides improved sealing upon insertion and retraction of a medical device both coaxially and non-coaxially, self-centering seal 222A reduces the risk of fluid leakage. Self-centering seal 222A provides superior leak prevention when medical device insertion is made off-axis and not directly on-plane with self-centering seal 222A or substantially parallel to a seal hub within which self-centering seal 222A is positioned. Plurality of raised curves 238A allow for additional side-to-side motion during insertion by flexing and allowing for additional movement of an inserted device without compromising sealing. As a result, self-centering seal 222A allows for easier device insertion, retention, and retraction within a guide sheath assembly.

Self-centering seal 222A provides a seal when nothing is inserted through a guide sheath assembly for low pressure venous systems, providing redundancy within a seal stack that includes, for example, a duckbill seal. Self-centering seal 222A also provides a seal when devices of various diameters (ranging from about 0.89 millimeter to about 8.13 millimeters) are inserted through self-centering seal 222A. As such, self-centering seal 222A can withstand the insertion and retraction of larger devices. Insertion and retraction of a medical device through self-centering seal 222A is easier and does not have to be as precise. As such, hemostasis loss is prevented during all portions of the medical procedure, including when no device is present in self-centering seal 222A, when a medical device is inserted into self-centering seal 222A, and when a medical device is withdrawn from self-centering seal 222A. Self-centering seal 222A is more successful at mitigating hemostasis loss for the patient and can be used in conjunction with a broader variety of medical devices and procedural configurations.

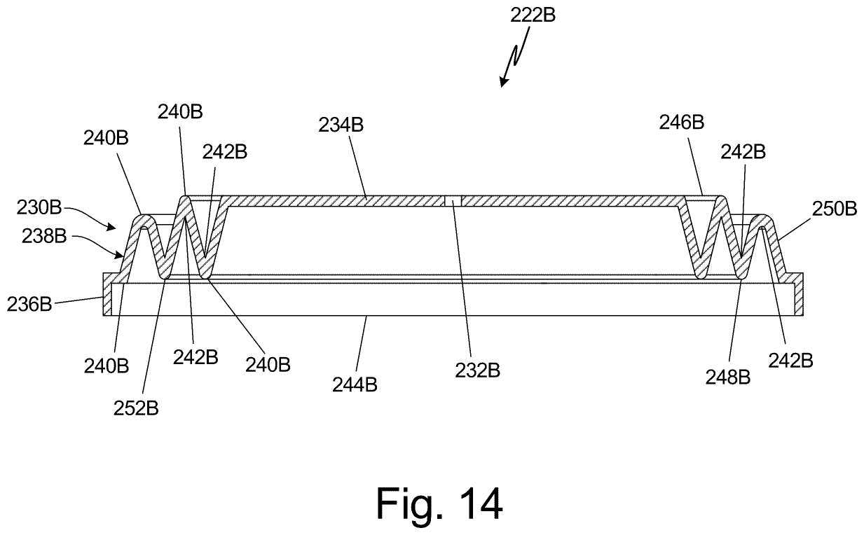

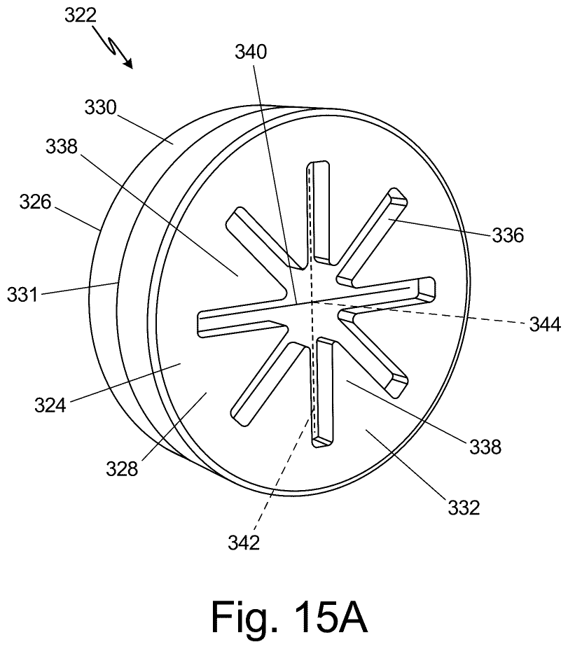

FIG. 14 is a cross-sectional side view of self-centering seal 222B. Self-centering seal 222B includes disc 230B, hole 232B, and central planar region 234B. Disc 230B includes rigid portion 236B, plurality of raised curves 238B (which include hills 240B and valleys 242B), distal end 244B, proximal end 246B, distal face 248B, and proximal face 250B. Plurality of raised curves 238B also include flattened region 252B.

Self-centering seal 222B has generally the same structure and function as self-centering seal 222A, shown in and described with respect to FIGS. 13A-13D. However, self-centering seal 222A has plurality of raised curves 238B, including hills 240B and valleys 242B, having different lengths. Plurality of raised curves 238B has two U-shaped concentric rings, the inner ring being longer than the outer ring, as seen in FIG. 14. In alternate embodiments, the outer ring may be longer than the inner ring. As such, the lengths of inner hill 240B and valley 242B are longer than the lengths of outer hill 240B and valley 242B. In alternate embodiments, the outer ring may be longer than the inner ring. Self-centering seal 222A having plurality of raised curves 238B with different lengths allows self-centering seal 222A to fit within various housing geometries.

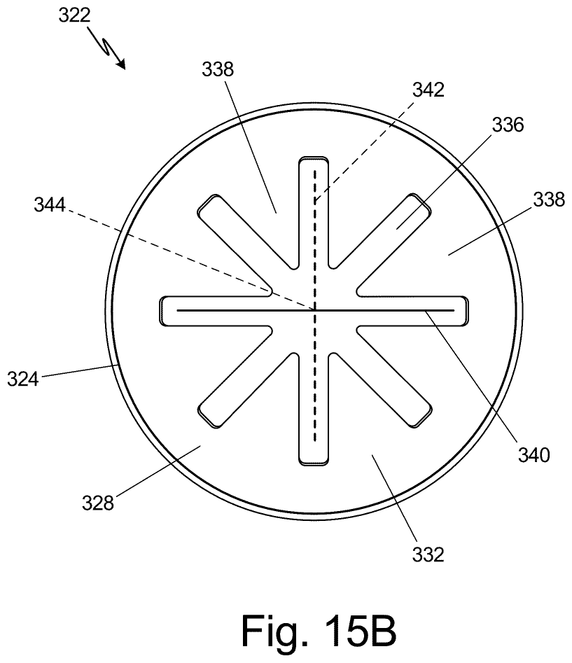

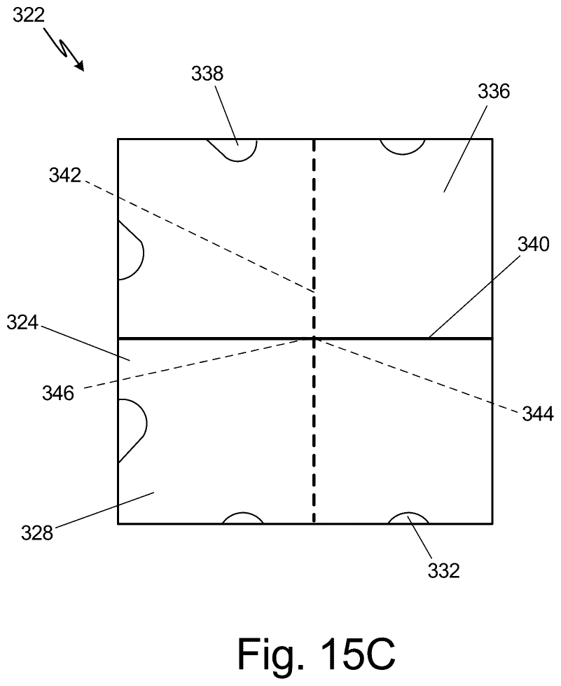

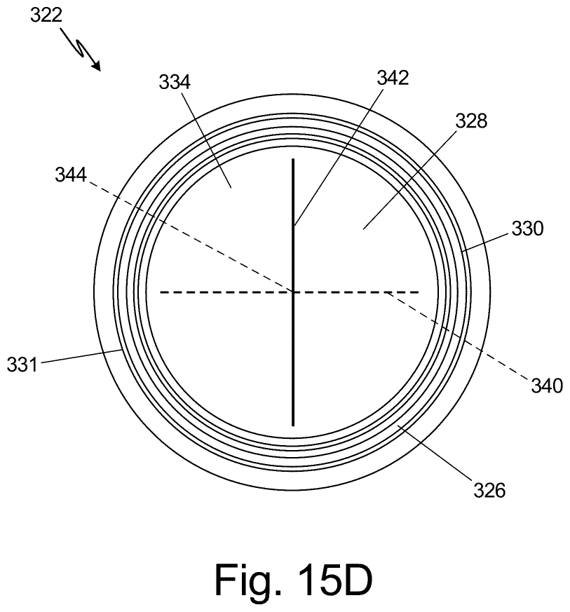

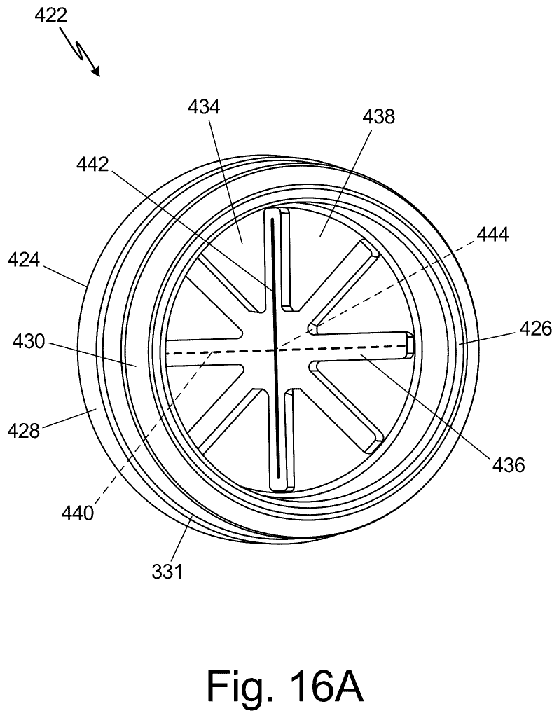

FIG. 15A is an orthogonal view of cross-plane seal 322 from first end 324 of cross-plane seal 322. FIG. 15B is an end view of cross-plane seal 322 from first end 324 of cross-plane seal 322. FIG. 15C is an enlarged end view of cross-plane seal 322 from first end 324 of cross-plane seal 322 showing hole 346 of cross-plane seal 322. FIG. 15D is an end view of cross-plane seal 322 from second end 326 of cross-plane seal 322. FIGS. 15A, 15B, 15C, and 15D will be discussed together.

Cross-plane seal 322 includes first end 324 (shown in FIGS. 15A-15C), second end 326 (shown in FIGS. 15A and 15D), disc 328, annular portion 330 (shown in FIGS. 15A and 15D), and step 331 (shown in FIGS. 15A and 15D). Disc 328 includes first face 332 (shown in FIGS. 15A-15C), second face 334 (shown in FIG. 15D), star-shaped cut out 336 (shown in FIGS. 15A-15C), triangular portions 338 (shown in FIGS. 15A-15C), first slit 340, second slit 342, intersection 344, and hole 346 (shown in FIG. 15C).

Cross-plane seal 322 is made of a liquid injection silicone, or other similar material, that is cured following molding to stabilize the material. Cross-plane seal 322 has a U-shaped cross-sectional profile with a closed first end 324 and an open second end 326. First end 324 may be the distal end or the proximal end of cross-plane seal 322. Second end 326 is the other end of cross-plane seal 322 opposite first end 324 such that second end 326 may be the proximal end or the distal end of cross-plane seal 322. Disc 328 and annular portion 330 make up a body of cross-plane seal 322. Disc 328 is at first end 324 of cross-plane seal 322. Disc 328 is cylindrical, having a thickness. For example, disc 328 can have a thickness of ⅛ inch (0.3175 centimeter). Disc 328 has a greater outer diameter than an outer diameter of annular portion 330. Annular portion 330 is connected to and extends from disc 328 to second end 326. Step 331 is formed by disc 328 having a greater outer diameter than the outer diameter of annular portion 330. Step 331 is formed where disc 328 is connected to annular portion 330 such that step 331 is between disc 328 and annular portion 330.

Disc 328 has first face 332 at the first side of disc 328 and second face 334 at the second side of disc 328. First face 332 is at first end 324 of cross-plane seal 322. Second face 334 is adjacent annular portion 330. Disc 328 has a thickness between first face 332 and second face 334. First face 332 has star-shaped cut out 336, which is a star-shaped recess in first face 332. As such, star-shaped cut out 336 has a smaller thickness than the portion of disc 328 without star-shaped cut out 336. In this example, star-shaped cut out 336 has a central region with eight lines, or rectangular arms, evenly spaced around and extending from the central region of star-shaped cut out 336. Triangular portions 338 are triangular-shaped, or wedge-shaped, portions of first face 332 between the lines, or arms, of star-shaped cut out 336. As such, triangular portions 338 are between recessed portions of star-shaped cut out 336 and have a thickness greater than star-shaped cut out 336. Star-shaped cut out 336 is positioned within first face 332 of disc 328 such that first slit 340 extends into and across star-shaped cut out 336 from a first arm to a second arm, and second slit 342 extends into and across star-shaped cut out 336 from a third arm to a fourth arm perpendicular to first slit 340.

First slit 340 is a slit having a partial through-cut into first face 332 of disc 328. First slit 340 is aligned with arms of star-shaped cut out 336. The through-cut of first slit 340 extends more than halfway, but not fully, through the thickness of disc 328. For example, first slit 340 may extend 3/32 inch (2.38 millimeters) deep through disc 328 having a material thickness of ⅛ inch (3.175 millimeters). In this example, first slit 340 may be ½ inch (12.7 millimeters) long within disc 328. First slit 340 can extend through between 50 percent and 100 percent of the thickness of disc 328, preferably at 75 percent of the thickness of disc 328. Second slit 342 is a slit having a partial through-cut into second face 334 of disc 328. Second slit 342 is aligned with arms of star-shaped cut out 336. The through-cut of second slit 342 extends more than halfway, but not fully, through the thickness of disc 328. For example, second slit 342 may extend 3/32 inch (2.38 millimeters) deep through disc 328 having a material thickness of ⅛ inch (3.175 millimeters). In this example, second slit 342 may be ½ inch (12.7 millimeters) long within disc 328. Second slit 342 can extend through between 50 percent and 100 percent of the thickness of disc 328, preferably at 75 percent of the thickness of disc 328. First slit 340 is perpendicular, or orthogonal, to second slit 342. Because first slit 340 extends more than halfway through disc 328 from first face 332, second slit 342 extends more than halfway through disc 328 from second face 334, and first slit 340 and second slit 342 are perpendicular, first slit 340 and second slit 342 partially overlap to form intersection 344. As such, intersection 344 is within disc 328 at a center of star-shaped cut out 336. Intersection 344 forms hole 346, an opening, or pathway, through disc 328 between first face 332 of disc 328 and second face 334 of disc 328 at a center of star-shaped cut out 336. As such, hole 346 consists of first slit 340, intersection 344, and second slit 342.

Cross-plane seal 322 may be used in place of cross-plane seal 22, cross-plane seal 122, or self-centering seal 222. Cross-plane seal 322 may have first end 324 or second end 326 adjacent to and contacting duckbill seal 20, as described with respect to FIGS. 2A, 2B, and 3. As such, second end 326 or first end 324 of cross-plane seal 322 may be adjacent to and contacting ring 24, with ring 24 being positioned within annular portion 330, as described with respect to FIGS. 2A, 2B, and 3. Thus, disc 328 may contact duckbill seal 20 or ring 24. Alternatively, cross-plane seal 322 may be used alone or with any combination of one or more seals. For example, a seal may be positioned adjacent second end 326 of cross-plane seal 322 to contact an end of annular portion 330, in place of disc seal 26 as described with respect to FIGS. 2A, 2B, and 3. In another example, cross-plane seal 322 may be the most proximal seal within a seal hub, such as seal hub 12, 112, or 212. The diameter and thickness of cross-plane seal 322, the depths of first slit 340 and second slit 342, and the number of arms of star-shaped cut out 336 can vary to any suitable configuration to accommodate various medical devices.

Cross-plane seal 322 seals on medical devices of various sizes, such as a small diameter guidewire, a large diameter loader, or any other suitable device. Star-shaped cut out 336 provides a lead-in for blunt-tipped devices, such as dilators and transcatheter devices, to guide the distal tips of the devices toward small hole 346 of cross-plane seal 322 when cross-plane seal 322 is the most proximal seal. As a medical device is inserted through cross-plane seal 322, the medical device exerts force on disc 328 of cross-plane seal 322. Intersection 344 of first slit 340 and second slit 342 within star-shaped cutout 336 is forced open to open hole 346 as cross-plane seal 322 elongates in response to the insertion force. Because star-shaped cut out 336 is thinner, or has a lesser thickness, than the rest of disc 328, devices can penetrate disc 328 more easily through star-shaped cut out 336. Star-shaped cut out 336 reduces the force required for seal penetration, particularly for large diameter devices. As triangular portions 338 are thicker than star-shaped cut out 336, triangular portions 338 provide structural stability to disc 328 of cross-plane seal 322.

The medical device is extended through hole 346 at intersection 344. When the medical device is positioned within cross-plane seal 322, first slit 340 and second slit 342 seal around the medical device. First slit 340 and second slit 342 are positioned perpendicular to each other and on opposite faces of disc 328 and within star-shaped cut out 336 to provide a constant sealing effect, regardless of whether the medical device is extending through cross-plane seal 322 coaxially. As such, the medical device may be coaxial or non-coaxial to cross-plane seal 322. Further, first slit 340 and second slit 342 each extend less than fully through disc 328, or are partial through-cut slits, to provide a sealing region that is also able to retain the medical device that is inserted coaxially or non-coaxially. When a medical device, such as a loader with a large outer diameter, is inserted into cross-plane seal 322, annular portion 330 of cross-plane seal 322 flexes and expands radially. Cross-plane seal 322 can retain a medical device that is inserted or retracted non-coaxially.

The medical device can be retracted through hole 346 at intersection 344 to remove the medical device from cross-plane seal 322. When the medical device is retracted, the material of cross-plane seal 322 and star-shaped cut out 336 lower the force caused by retraction. Triangular portions 338 are thicker than star-shaped cut out 336 to provide for the return of disc 328 to its original orientation. First slit 340 and second slit 342 return to their original positions, and hole 346 at intersection 344 of disc 328 shrinks to close. Because first slit 340 and second slit 342 only partially overlap, cross-plane seal 322 provides an effective seal when no device is present within cross-plane seal 322. Additionally, cross-plane seal 322 is less likely to cave or fold in on itself, easing insertion and retraction of medical devices into and from cross-plane seal 322.

When cross-plane seal 322 is positioned within a seal hub, such as seal hub 12, 112, or 212, the outer diameter of disc 328 of cross-plane seal 322 radially seals against the seal hub. The outer diameter of disc 328 forms a sealing surface that has an interference fit within the seal hub. As such, the outer diameter of disc 328 abuts and seals against the inner diameter of the seal hub, which provides a seal between the seal hub and cross-plane seal 322. The outer diameter of disc 328 of cross-plane seal 322 also interacts with the seal hub to provide retention within the seal hub. Cross-plane seal 322 is made of a flexible material that lowers insertion and retraction pressure. Disc 328 provides radial structure to flexible cross-plane seal 322, allowing for axial and radial compression within the seal hub to prevent para-seal leak paths. Space within the seal hub is also available for annular portion 330 of cross-plane seal 322 to expand radially due to the smaller outer diameter of annular portion 330. As cross-plane seal 322 distorts, the counterforce of the insertion is minimized, helping to retain cross-plane seal 322 within the seal hub.

Cross-plane seal 322 provides a seal when nothing is inserted through cross-plane seal 322 and when devices of various diameters are inserted through cross-plane seal 322. Cross-plane seal 322 seals on small diameter devices, such as a guidewire, and provides low insertion forces, which prevents damage when larger diameter devices, such as a dilator, are inserted through cross-plane seal 322. The reduced insertion force achieved by cross-plane seal 322 allows for easier insertion, retention, and retraction of a medical device and enables cross-plane seal 322 to more easily withstand insertion and retraction of larger devices. As such, cross-plane seal 322 prevents hemostasis loss with both small diameter and large diameter devices. Further, hemostasis loss is prevented during all portions of the medical procedure, including when nothing is inserted through cross-plane seal 322, when a small medical device is inserted through cross-plane seal 322, when a large medical device is inserted through cross-plane seal 322, and when a medical device is withdrawn from cross-plane seal 322. Because cross-plane seal 322 provides improved sealing upon insertion and retraction of a medical device both coaxially and non-coaxially, cross-plane seal 322 further reduces the risk of fluid leakage. Additionally, because star-shaped cut out 336 acts as a visual target, the medical device is more likely to reach a center of cross-plane seal 322 and exit cross-plane seal 322 in the proper position. Cross-plane seal 322 is also shaped to keep the medical device centered. As a result, the medical device is less likely to cause a tear in cross-plane seal 322, which can lead to hemostasis failure and/or detached particulate being introduced into the bloodstream. Cross-plane seal 322 is more successful at mitigating hemostasis loss for the patient and can be used in conjunction with a broader variety of medical devices and procedural configurations.

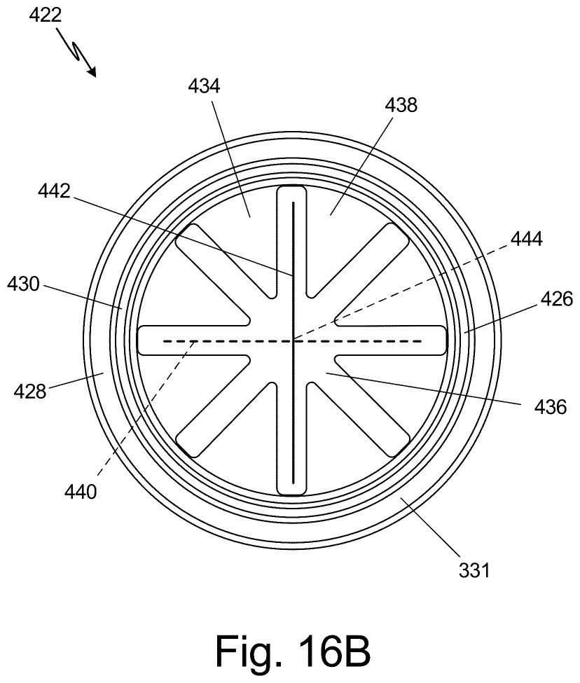

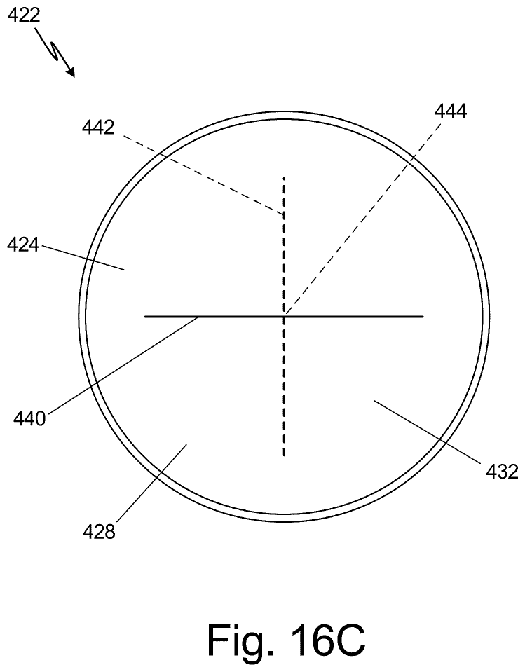

FIG. 16A is an orthogonal view of cross-plane seal 422 from second end 426 of cross-plane seal 422. FIG. 16B is an end view of cross-plane seal 422 from second end 426 of cross-plane seal 422. FIG. 16C is an end view of cross-plane seal 422 from first end 424 of cross-plane seal 422. FIGS. 16A, 16B, and 16C will be discussed together.

Cross-plane seal 422 includes first end 424 (shown in FIGS. 16A and 16C), second end 426 (shown in FIGS. 16A and 16B), disc 428, annular portion 430 (shown in FIGS. 16A and 16B), and step 431 (shown in FIGS. 16A and 16B). Disc 428 includes first face 432 (shown in FIG. 16C), second face 434 (shown in FIGS. 16A and 16B), star-shaped cut out 436 (shown in FIGS. 16A and 16B), triangular portions 438 (shown in FIGS. 16A and 16B), first slit 440, second slit 442, and intersection 444.

Cross-plane seal 422 is made of a liquid injection silicone, or other similar material, that is cured following molding to stabilize the material. Cross-plane seal 422 has a U-shaped cross-sectional profile with a closed first end 424 and an open second end 426. First end 424 may be the distal end or the proximal end of cross-plane seal 422. Second end 426 is the other end of cross-plane seal 422 opposite first end 424 such that second end 426 may be the proximal end or the distal end of cross-plane seal 422. Disc 428 and annular portion 430 make up a body of cross-plane seal 422. Disc 428 is at first end 424 of cross-plane seal 422. Disc 428 is cylindrical, having a thickness. For example, disc 428 can have a thickness of ⅛ inch (0.3175 centimeter). Disc 428 has a greater outer diameter than an outer diameter of annular portion 430. Annular portion 430 is connected to and extends from disc 428 to second end 426. Step 431 is formed by disc 428 having a greater outer diameter than the outer diameter of annular portion 430. Step 431 is formed where disc 428 is connected to annular portion 430 such that step 431 is between disc 428 and annular portion 430.

Disc 428 has first face 432 at the first side of disc 428 and second face 434 at the second side of disc 428. First face 432 is at first end 424 of cross-plane seal 422. Disc 428 has a thickness between first face 432 and second face 434. Second face 434 is adjacent annular portion 430. Second face 434 has star-shaped cut out 436, which is a star-shaped recess in second face 434, such that star-shaped cut out 436 is adjacent annular portion 430. As such, star-shaped cut out 436 has a smaller thickness than the portion of disc 428 without star-shaped cut out 436. In this example, star-shaped cut out 436 has a central region with eight lines, or rectangular arms, evenly spaced around and extending from the central region of star-shaped cut out 436. Triangular portions 438 are triangular-shaped, or wedge-shaped, portions of second face 434 between the lines, or arms, of star-shaped cut out 436. As such, triangular portions 438 are between recessed portions of star-shaped cut out 436 and have a thickness greater than star-shaped cut out 436. Star-shaped cut out 436 is positioned within second face 434 of disc 428 such that first slit 440 extends into and across star-shaped cut out 436 from a first arm to a second arm, and second slit 442 extends into and across star-shaped cut out 436 from a third arm to a fourth arm perpendicular to first slit 440.

First slit 440 is a slit having a partial through-cut into first face 432 of disc 428. First slit 440 is aligned with arms of star-shaped cut out 436. The through-cut of first slit 440 extends more than halfway, but not fully, through the thickness of disc 428. For example, first slit 440 may extend 3/32 inch (2.38 millimeters) deep through disc 428 having a material thickness of ⅛ inch (3.175 millimeters). In this example, first slit 440 may be ½ inch (12.7 millimeters) long within disc 428. First slit 440 can extend through between 50 percent and 100 percent of the thickness of disc 428, preferably at 75 percent of the thickness of disc 428. Second slit 442 is a slit having a partial through-cut into second face 434 of disc 428. Second slit 442 is aligned with arms of star-shaped cut out 436. The through-cut of second slit 442 extends more than halfway, but not fully, through the thickness of disc 428. For example, second slit 442 may extend 3/32 inch (2.38 millimeters) deep through disc 428 having a material thickness of ⅛ inch (3.175 millimeters). In this example, second slit 442 may be ½ inch (12.7 millimeters) long within disc 428. Second slit 442 can extend through between 50 percent and 100 percent of the thickness of disc 428, preferably at 75 percent of the thickness of disc 428. First slit 440 is perpendicular, or orthogonal, to second slit 442. Because first slit 440 extends more than halfway through disc 428 from first face 432, second slit 442 extends more than halfway through disc 428 from second face 434, and first slit 440 and second slit 442 are perpendicular, first slit 440 and second slit 442 partially overlap to form intersection 444. As such, intersection 444 is within disc 428 at a center of star-shaped cut out 436. Intersection 444 forms a hole, which is an opening, or pathway, through disc 428 between first face 432 of disc 428 and second face 434 of disc 428 at a center of star-shaped cut out 436. As such, the hole consists of first slit 440, intersection 444, and second slit 442.

Cross-plane seal 422 may be used in place of cross-plane seal 22, cross-plane seal 122, or self-centering seal 222. Cross-plane seal 422 may have first end 424 or second end 426 adjacent to and contacting duckbill seal 20, as described with respect to FIGS. 2A, 2B, and 3. As such, second end 426 or first end 424 of cross-plane seal 422 may be adjacent to and contacting ring 24, with ring 24 being positioned within annular portion 430, as described with respect to FIGS. 2A, 2B, and 3. Thus, disc 428 may contact duckbill seal 20 or ring 24. Alternatively, cross-plane seal 422 may be used alone or with any combination of one or more seals. For example, a seal may be positioned adjacent second end 426 of cross-plane seal 422 to contact an end of annular portion 430, in place of disc seal 26 as described with respect to FIGS. 2A, 2B, and 3. In another example, cross-plane seal 422 may be the most proximal seal within a seal hub, such as seal hub 12, 112, or 212. The diameter and thickness of cross-plane seal 422, the depths of first slit 440 and second slit 442, and the number of arms of star-shaped cut out 436 can vary to any suitable configuration to accommodate various medical devices.

Cross-plane seal 422 seals on medical devices of various sizes, such as a small diameter guidewire, a large diameter loader, or any other suitable device. Star-shaped cut out 436 provides a lead-in for blunt-tipped devices, such as dilators and transcatheter devices, to guide the distal tips of the medical devices toward the small hole of cross-plane seal 422 when cross-plane seal 422 is the most proximal seal. As a medical device is inserted through cross-plane seal 422, the medical device exerts force on disc 428 of cross-plane seal 422. Intersection 444 of first slit 440 and second slit 442 within star-shaped cutout 436 is forced open to form the hole as cross-plane seal 422 elongates in response to the insertion force. Because star-shaped cut out 436 is thinner, or has a lesser thickness, than the rest of disc 428, devices can penetrate disc 428 more easily through star-shaped cut out 436. Star-shaped cut out 436 reduces the force of seal penetration by large diameter devices. As triangular portions 438 are thicker than star-shaped cut out 436, triangular portions 438 provide structural stability to disc 428 of cross-plane seal 422.

The medical device is extended through the hole at intersection 444. When the medical device is positioned within cross-plane seal 422, first slit 440 and second slit 442 seal around the medical device. First slit 440 and second slit 442 are positioned perpendicular to each other and on opposite faces of disc 428 and within star-shaped cut out 436 to provide a constant sealing effect, regardless of whether the medical device is extending through cross-plane seal 422 coaxially. As such, the medical device may be coaxial or non-coaxial to cross-plane seal 422. Further, first slit 440 and second slit 442 each extend less than fully through disc 428, or are partial through-cut slits, to provide a sealing region that is also able to retain the medical device that is inserted coaxially or non-coaxially. When a medical device, such as a loader with a large outer diameter, is inserted into cross-plane seal 422, annular portion 430 of cross-plane seal 422 flexes and expands radially. The retention ability of cross-plane seal 422 is especially important for a medical device that is inserted or retracted non-coaxially.

The medical device can be retracted through the hole at intersection 444 to remove the medical device from cross-plane seal 422. When the medical device is retracted, the material of cross-plane seal 422 and star-shaped cut out 436 lower the force caused by retraction. Triangular portions 438 are thicker than star-shaped cut out 436 to more easily provide for the return of disc 428 to its original orientation. First slit 440 and second slit 442 return to their original positions, and the hole at intersection 444 of disc 428 shrinks to close. Because first slit 440 and second slit 442 only partially overlap, cross-plane seal 422 provides an effective seal when no device is present within cross-plane seal 422. Additionally, cross-plane seal 422 is less likely to cave or fold in on itself, easing insertion and retraction of medical devices into and from cross-plane seal 422.

When cross-plane seal 422 is positioned within a seal hub, such as seal hub 12, 112, or 212, the outer diameter of disc 428 of cross-plane seal 422 radially seals against the seal hub. The outer diameter of disc 428 forms a sealing surface that has an interference fit within the seal hub. As such, the outer diameter of disc 428 abuts and seals against the inner diameter of the seal hub, which provides a seal between the seal hub and cross-plane seal 422. The outer diameter of disc 428 of cross-plane seal 422 also interacts with the seal hub to provide retention within the seal hub. Cross-plane seal 422 is made of a flexible material that lowers insertion and retraction pressure. Disc 428 provides radial structure to flexible cross-plane seal 422, allowing for axial and radial compression within the seal hub to prevent para-seal leak paths. Space within the seal hub is also available for annular portion 430 of cross-plane seal 422 to expand radially due to the smaller outer diameter of annular portion 430. As cross-plane seal 422 distorts, the counterforce of the insertion is minimized, helping to retain cross-plane seal 422 within the seal hub.

Cross-plane seal 422 provides a seal when nothing is inserted through cross-plane seal 422 and when devices of various diameters are inserted through cross-plane seal 422. Cross-plane seal 422 seals on small diameter devices, such as a guidewire, and provides low insertion forces, which prevents damage when larger diameter devices, such as a dilator, are inserted through cross-plane seal 422. The reduced insertion force achieved by cross-plane seal 422 allows for easier insertion, retention, and retraction of a medical device and enables cross-plane seal 422 to withstand insertion and retraction of larger devices. As such, cross-plane seal 422 prevents hemostasis loss with both small diameter and large diameter devices. Further, hemostasis loss is prevented during all portions of the medical procedure, including when nothing is inserted through cross-plane seal 422, when a small medical device is inserted through cross-plane seal 422, when a large medical device is inserted through cross-plane seal 422, and when a medical device is withdrawn from cross-plane seal 422. Because cross-plane seal 422 provides improved sealing upon insertion and retraction of a medical device both coaxially and non-coaxially, cross-plane seal 422 reduces the risk of fluid leakage. Cross-plane seal 422 is also shaped to keep the medical device centered. As a result, the medical device is less likely to cause a tear in cross-plane seal 422, which can lead to hemostasis failure and/or detached particulate being introduced into the bloodstream. Cross-plane seal 422 is more successful at mitigating hemostasis loss for the patient and can be used in conjunction with a broader variety of medical devices and procedural configurations.

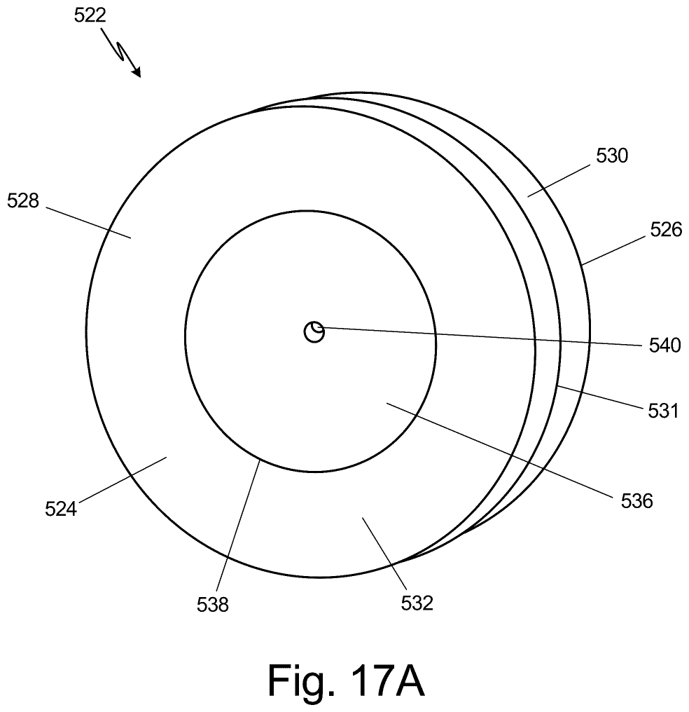

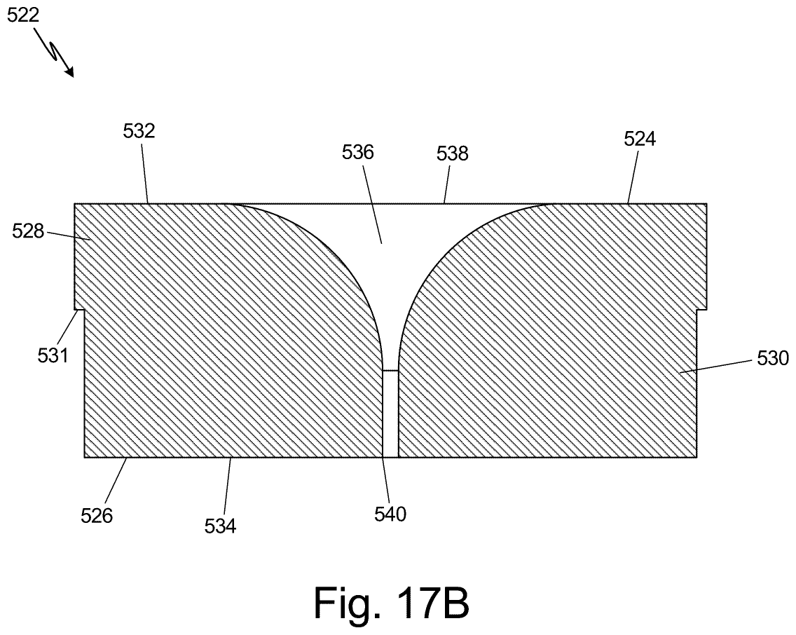

FIG. 17A is a perspective view of funnel seal 522 from proximal end 524 of funnel seal 522. FIG. 17B is a cross-sectional side view of funnel seal 522. FIGS. 17A and 17B will be discussed together.

Funnel seal 522 includes proximal end 524, distal end 526, disc 528, annular portion 530, step 531, proximal face 532, distal face 534 (shown in FIG. 17B), funnel portion 536, first hole 538, and second hole 540.

Funnel seal 522 is made of a material, such as silicone, having a lower medium durometer, such that the funnel seal 522 is flexible. The durometer of funnel seal 522 is selected based on the needs of the application of funnel seal 522, such as the sizes of the medical devices that will be inserted through funnel seal 522. For example, the material of funnel seal 522 may have a durometer of about 25 A. Funnel seal 522 has proximal end 524 at a first end of funnel seal 522 and distal end 526 at the other, second end of funnel seal 522 opposite proximal end 524. Disc 528 and annular portion 530 make up a body of funnel seal 522. Disc 528 is at proximal end 524 of funnel seal 522. Disc 528 is cylindrical, having a first thickness. For example, disc 528 can have a thickness of ⅛ inch (0.3175 centimeter). Disc 528 has a greater outer diameter than an outer diameter of annular portion 530. Annular portion 530 is a cylindrical portion connected to and extending from disc 528 to distal end 526 of funnel seal 522. Annular portion 530 has a second thickness. The second thickness of annular portion 530 may be greater than the first thickness of disc 528. In alternate examples, the second thickness of annular portion 530 may be lesser than the first thickness of disc 528. The overall thickness of funnel seal 522, including disc 528 and annular portion 530, may be about 1 centimeter. Step 531 is formed by disc 528 having a greater outer diameter than the outer diameter of annular portion 530. Step 531 is formed where disc 528 is connected to annular portion 530 such that step 531 is between disc 528 and annular portion 530. In alternate examples, funnel seal 522 may not include step 531, and disc 528 and annular portion 530 may have the same outer diameter.

Proximal face 532 is a face, or surface, of proximal end 524 of funnel seal 522. As such, proximal face 532 of funnel seal 522 is a proximal face of disc 528. Distal face 534 is a face, or surface, of distal end 526 of funnel seal 522. As such, distal face 534 of funnel seal 522 is a distal face of annular portion 530. Funnel portion 536 is a funnel-shaped opening, or space, that extends from proximal face 532 of proximal end 524 to distal face 534 of distal end 526. As such, funnel portion 536 is an opening, or pathway through funnel seal 522. Funnel portion 536 decreases in cross-section, or narrows, from proximal end 524 to distal end 526 of funnel seal 522. As such, funnel portion 536 is defined by a series of circles that decrease in diameter. First hole 538 is an opening in proximal face 532 of funnel seal 522 and forms a proximal end of funnel portion 536. Second hole 540 is an opening in distal face 534 of funnel seal 522 and forms a distal end of funnel portion 536. First hole 538 has a diameter that is larger than a diameter second hole 540. Funnel portion 536 extends from first hole 538, through disc 528 and annular portion 530, to second hole 540 at distal end 526 of funnel seal 522.

Funnel seal 522 may be used in place of cross-plane seal 22, cross-plane seal 122, or self-centering seal 222. As such, funnel seal 522 may have distal end 526 adjacent to and contacting duckbill seal 20, as described with respect to FIGS. 2A, 2B, and 3. Proximal end 524 of funnel seal 522 may be adjacent to and contacting disc seal 26, with ring 24 not being included in seal stack 14, as described with respect to FIGS. 2A, 2B, and 3. Alternatively, funnel seal 522 may be used alone or with any combination of one or more seals. For example, funnel seal 522 may be the most proximal seal within a seal hub, such as seal hub 12, 112, or 212. The diameter and thickness of funnel seal 522, the shape and curvature of funnel portion 536, and the material of funnel seal 522 can vary to result in any suitable configuration to accommodate various medical devices. For example, the durometer of funnel seal 522 should be high enough to accommodate smaller guidewires but also low enough to accommodate larger dilators and devices.

Funnel seal 522 seals on medical devices of various sizes, such as a small diameter guidewire, a large diameter loader, or any other suitable device. Funnel portion 536 provides a visual target for insertion of a medical device through funnel seal 522 when funnel seal 522 is the most proximal seal. The medical device is extended through first hole 538. As the medical device enters funnel portion 536 via larger first hole 538, funnel portion 536 guides the distal tip of the medical device from first hole 538 to second hole 540 of funnel seal 522. At least a portion of funnel portion 536 is forced open, widening second hole 540, as funnel seal 522 elongates in response to the insertion force. Funnel portion 536 reduces the force of seal penetration by large diameter devices. At least a distal portion of annular portion 530 seals around the medical device and provides support to the inserted medical device. Additionally, the drag from the insertion of the medical device through the distal portion of funnel portion 536, or second hole 540, will cause a proximal surface defining funnel portion 536 to roll inward and hug the shaft of the inserted medical device, providing additional surface area for sealing against the medical device.

The medical device can be retracted from second hole 540 through funnel portion 536 and out of first hole 538 to remove the medical device from funnel seal 522. When the medical device is retracted, the material of funnel seal 522 and the shape of funnel portion 536 lower the force caused by retraction. Because funnel seal 522 is manufactured by molding funnel seal 522 first and subsequently creating second hole 540 in funnel seal 522 in a separate step by separating the material of funnel seal 522 without removing material (e.g. by slitting or cutting with a pin, needle, or blade), funnel seal 522 recovers back to its original molded position to seal completely when a medical device is retracted. As such, funnel seal 522 also provides an effective seal when no device is present within funnel seal 522.

When funnel seal 522 is positioned within a seal hub, such as seal hub 12, 112, or 212, the outer diameter of disc 528 of funnel seal 522 radially seals against the seal hub. The outer diameter of disc 528 forms a sealing surface that has an interference fit within the seal hub. As such, the outer diameter of disc 528 abuts and seals against the inner diameter of the seal hub, which provides a seal between the seal hub and funnel seal 522. The outer diameter of disc 528 of funnel seal 522 also interacts with the seal hub to provide retention within the seal hub. Further, disc 528 provides support for funnel portion 536 of funnel seal 522. Funnel seal 522 is made of a flexible material that lowers insertion and retraction forces. Disc 528 provides radial structure to flexible funnel seal 522, allowing for axial and radial compression within the seal hub to prevent leak paths. Space within the seal hub is also available for annular portion 530 of funnel seal 522 to expand radially due to the smaller outer diameter of annular portion 530. As funnel seal 522 distorts, the counterforce of the insertion is minimized, helping to retain funnel seal 522 in the proper position within the seal hub.

Funnel portion 536 helps keep the medical device at a center of funnel seal 522 as the medical device is being inserted through funnel seal 522. As a result, the medical device is less likely to cause a tear in funnel seal 522, which can lead to hemostasis failure and/or detached particulate being introduced into the bloodstream. Because funnel portion 536 acts as a guide, the medical device is more likely to reach a center of distal end 526 of funnel seal 522, which is at second hole 540, and exit funnel seal 522 in the proper position.

Funnel seal 522 provides a seal when nothing is inserted through funnel seal 522 and when devices of various diameters are inserted through funnel seal 522. Funnel seal 522 can accommodate a larger array of medical devices while reducing the risk of hemostasis loss. Funnel seal 522 seals on small diameter devices, such as a guidewire, and provides low insertion forces, which prevents damage when larger diameter devices, such as a dilator, are inserted through funnel seal 522. As such, funnel seal 522 prevents hemostasis loss with both small diameter and large diameter devices. Further, the reduced insertion force achieved by funnel seal 522 allows for easier insertion, retention, and retraction of a medical device and enables funnel seal 522 to withstand insertion and retraction of larger devices. As such, hemostasis loss is prevented during all portions of the medical procedure, including when nothing is inserted through funnel seal 522, when a small medical device is inserted through funnel seal 522, when a large medical device is inserted through funnel seal 522, and when a medical device is withdrawn from funnel seal 522. Because funnel seal 522 provides improved sealing upon insertion and retraction of a medical device, funnel seal 522 reduces the risk of fluid leakage. Funnel seal 522 is more successful at mitigating hemostasis loss for the patient and can be used in conjunction with a broader variety of medical devices and procedural configurations.

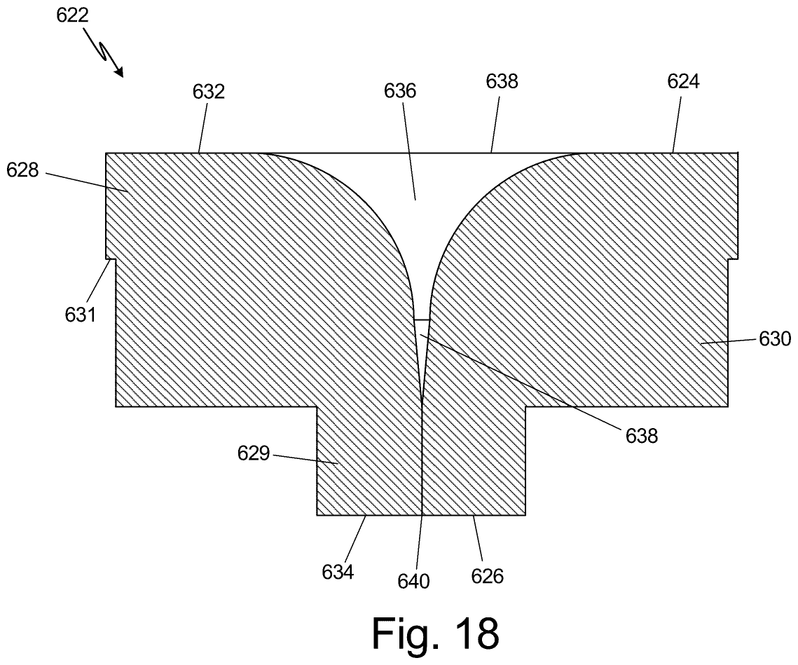

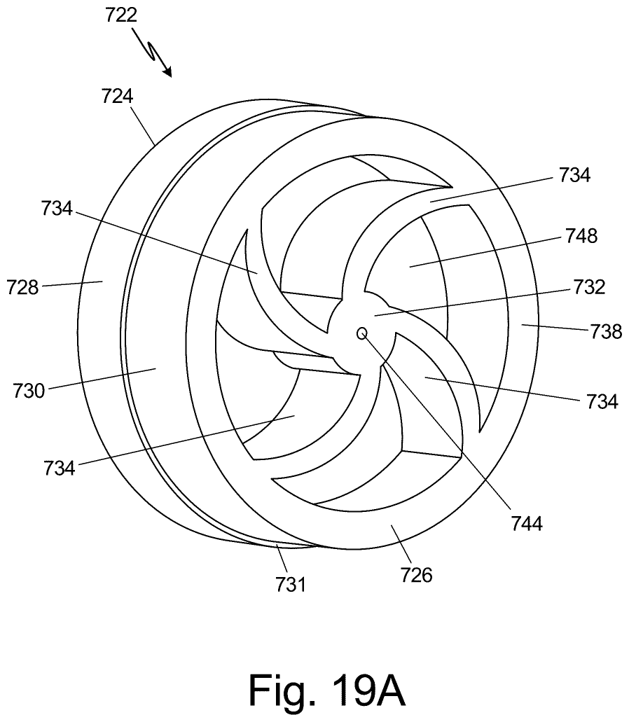

FIG. 18 is a cross-sectional side view of funnel seal 622. Funnel seal 622 includes proximal end 624, distal end 626, disc 628, ring portion 629, annular portion 630, step 631, proximal face 632, distal face 634, funnel portion 636, first hole 638, and second hole 640.

Funnel seal 622, shown in FIG. 18, has generally the same structure and function as funnel seal 522, shown in and described with respect to FIGS. 17A and 17B. However, funnel seal 622 also includes ring portion 629, which forms distal end 626 of funnel seal 622. As such, ring portion 629 forms distal face 634 of funnel seal 622, which has second hole 640. A proximal end of ring portion 629 is connected to a distal end of annular portion 630, annular portion 630 being between disc 628 and ring portion 629. Funnel portion 636 extends from first hole 638, through disc 628, annular portion 630, and ring portion 629, to second hole 640 at distal end 626 of funnel seal 622. An inner diameter of annular portion 630 contacts and seals against a medical device that is inserted through funnel seal 622.

While ring portion 629 may require additional insertion force, ring portion 629 provides additional sealing against and support to the inserted medical device, which helps prevent leakage and keep the inserted medical device in place, particularly smaller devices like guidewires. Ring portion 629 also provides additional linear guidance by elongating a distal end of funnel portion 636 to keep the inserted medical device at a center of funnel seal 622. Additionally, ring portion 629 of funnel seal 622 confines the inserted medical device, retaining the inserted medical device and preventing the inserted medical device from drifting away from the center of funnel seal 622. As such, funnel seal 622 is more successful at mitigating hemostasis loss for the patient, particularly with smaller medical devices, such as a guidewire.

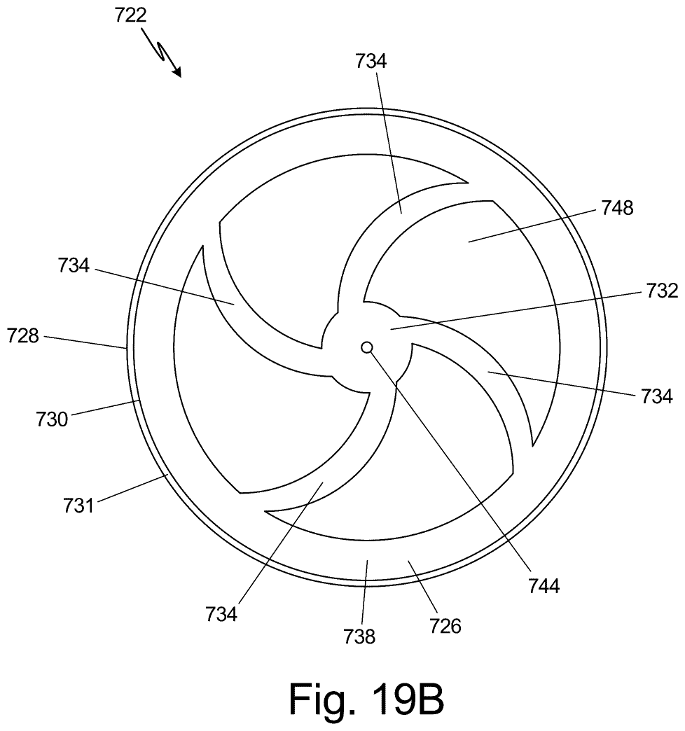

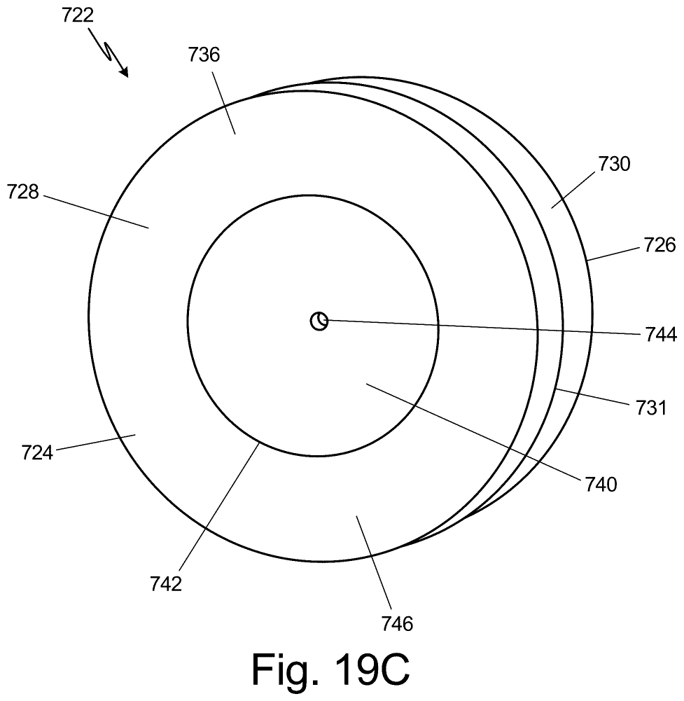

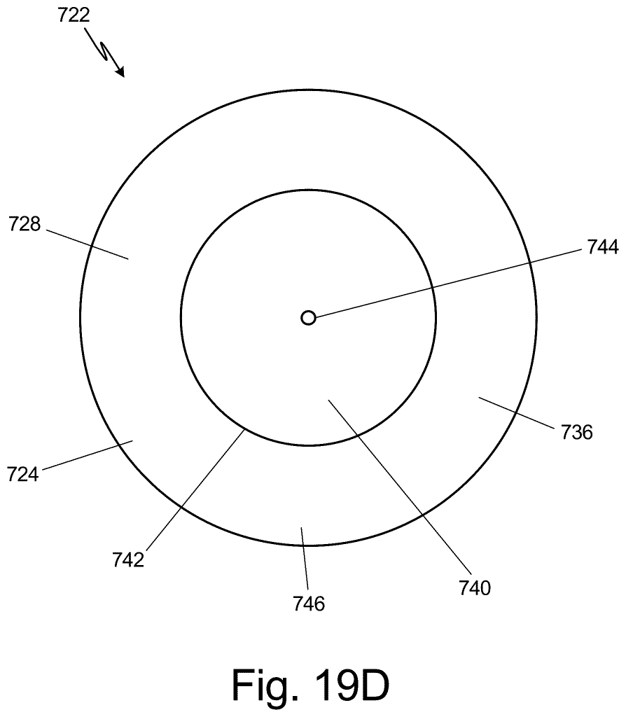

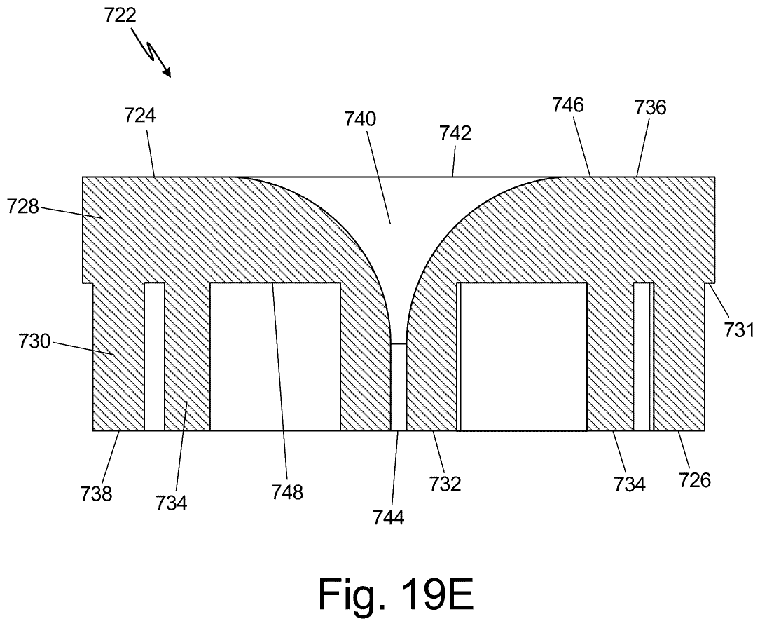

FIG. 19A is a perspective view of funnel seal 722 from distal end 726 showing curved ribs 734 of funnel seal 722. FIG. 19B is an end view of funnel seal 722 from distal end 726 of funnel seal 722 showing curved ribs 734 of funnel seal 722. FIG. 19C is a perspective view of funnel seal 722 from proximal end 724 of funnel seal 722. FIG. 19D is an end view of funnel seal 722 from proximal end 724 of funnel seal 722. FIG. 19E is a cross-sectional side view of funnel seal 722. FIGS. 19A, 19B, 19C, 19D, and 19E will be discussed together.

Funnel seal 722 includes proximal end 724 (shown in FIGS. 19A, 19C, 19D, and 19E), distal end 726 (shown in FIGS. 19A, 19B, 19C, and 19E), disc 728, annular portion 730 (shown in FIGS. 19A, 19B, 19C, and 19E), step 731 (shown in FIGS. 19A, 19B, 19C, and 19E), cylindrical portion 732 (shown in FIGS. 19A, 19B, and 19E), and curved ribs 734 (shown in FIGS. 19A, 19B, and 19E), proximal face 736 (shown in FIGS. 19C, 19D, and 19E), distal face 738 (shown in FIGS. 19A, 19B, and 19E), funnel portion 740 (shown in FIGS. 19C, 19D, and 19E), first hole 742 (shown in FIGS. 19C, 19D, and 19E), and second hole 744. Disc 728 includes proximal face 746 (shown in FIGS. 19C, 19D, and 19E), and distal face 748 (shown in FIGS. 19A, 19B, and 19E).

Funnel seal 722 is made of a material, such as silicone, having a lower medium durometer. The durometer of funnel seal 722 is selected based on the needs of the application of funnel seal 722, such as the sizes of the medical devices that will be inserted through funnel seal 722. For example, the material of funnel seal 722 may have a durometer of about 25 A. Funnel seal 722 has proximal end 724 at a first end of funnel seal 722 and distal end 726 at the other, second end of funnel seal 722 opposite proximal end 724. Disc 728 and annular portion 730 make up a body of funnel seal 722. Disc 728 is at proximal end 724 of funnel seal 722. Disc 728 is cylindrical, having a first thickness. For example, disc 728 can have a thickness of ⅛ inch (0.3175 centimeter). Disc 728 has a greater outer diameter than an outer diameter of annular portion 730. Annular portion 730 is a ring-like portion connected to and extending from disc 728 to distal end 726 of funnel seal 722. Annular portion 730 has a second thickness. The second thickness of annular portion 730 may be greater than the first thickness of disc 728. In alternate examples, the second thickness of annular portion 730 may be lesser than the first thickness of disc 728. The overall thickness of funnel seal 722, including disc 728 and annular portion 730, may be about 1 centimeter. Step 731 is formed by disc 728 having a greater outer diameter than the outer diameter of annular portion 730. Step 731 is formed where disc 728 is connected to annular portion 730 such that step 731 is between disc 728 and annular portion 730. In alternate examples, funnel seal 722 may not include step 731, and disc 728 and annular portion 730 may have the same outer diameter.

Cylindrical portion 732 is a cylindrical portion extending from a central portion of disc 728. Cylindrical portion 732 is spaced from and concentric with annular portion 730. Curved ribs 734 are curved portions extending from an outer diameter, or outer surface, of cylindrical portion 732 to an inner diameter, or inner surface, of annular portion 730. As such, curved ribs 734 extend across a space between cylindrical portion 732 and annular portion 730. Funnel seal 722 includes a plurality of curved ribs 734. In this example, funnel seal 722 includes four curved ribs 734. In alternate examples, funnel seal 722 may include any number of curved ribs 734 greater than two.

Proximal face 736 is a face, or surface, of proximal end 724 of funnel seal 722. As such, proximal face 736 of funnel seal 722 is a proximal face of disc 728. Distal face 738 is a face, or surface, of distal end 726 of funnel seal 722. As such, distal face 738 of funnel seal 722 is a distal face of annular portion 730 and cylindrical portion 732. Funnel portion 740 is a funnel-shaped opening, or space, that extends from proximal face 736 of proximal end 724 to distal face 738 of distal end 726. As such, funnel portion 740 is an opening, or pathway through funnel seal 722 that extends from first hole 742, through disc 728 and cylindrical portion 732, to second hole 744. Funnel portion 740 decreases in cross-section, or narrows, from proximal end 724 to distal end 726 of funnel seal 722. As such, funnel portion 740 is defined by a series of circles that decrease in diameter. First hole 742 is an opening in proximal face 736 of funnel seal 722 and forms a proximal end of funnel portion 740. Second hole 744 is an opening in distal face 748 of funnel seal 522 and forms a distal end of funnel portion 740. First hole 742 has a larger diameter than second hole 744. Funnel portion 740 extends from first hole 742, through disc 728 and annular portion 730, to second hole 544 at distal end 726 of funnel seal 522.

Disc 728 has proximal face 746 at the proximal end of disc 728 and distal face 748 at the distal end of disc 728. As such, proximal face 746 of disc 728 is at proximal face 736 of funnel seal 722. Proximal ends of curved ribs 734 are connected to distal face 748 of disc 728.

Funnel seal 722 may be used in place of cross-plane seal 22, cross-plane seal 122, or self-centering seal 222. As such, funnel seal 722 may have distal end 726 adjacent to and contacting duckbill seal 20, as described with respect to FIGS. 2A, 2B, and 3. Proximal end 724 of funnel seal 722 may be adjacent to and contacting disc seal 26, with ring 24 not being included in seal stack 14, as described with respect to FIGS. 2A, 2B, and 3. Alternatively, funnel seal 722 may be used alone or with any combination of one or more seals. For example, funnel seal 722 may be the most proximal seal within a seal hub, such as seal hub 12, 112, or 212. The diameter and thickness of funnel seal 722, the shape and curvature of funnel portion 740, the curvature and number of curved ribs 734, and the material of funnel seal 722 can vary to result in any suitable configuration to accommodate various medical devices. For example, the durometer of funnel seal 722 should be high enough to accommodate smaller guidewires but also low enough to accommodate larger dilators and devices.

Funnel seal 722 seals on medical devices of various sizes, such as a small diameter guidewire, a large diameter loader, or any other suitable device. Funnel portion 740 provides a visual target for insertion of a medical device through funnel seal 722 when funnel seal 722 is the most proximal seal. The medical device is extended through first hole 742. As the medical device enters funnel portion 740 via larger first hole 742, funnel portion 740 guides the distal tip of the medical device from first hole 742 to second hole 744 of funnel seal 722. At least a portion of funnel portion 740 is forced open, widening second hole 744, as funnel seal 722 elongates in response to the insertion force. Funnel portion 740 reduces the force of seal penetration by large diameter devices. Curved ribs 734 provide additional support to keep the medical device at the center of funnel seal 722 during insertion. As cylindrical portion 732 expands during the insertion of a medical device, curved ribs 734 buckle in a controlled, predefined circumferential direction that allows for an even distribution of force to keep the medical device centered within funnel seal 722. As such, the curvature of curved ribs 734 tunes the insertion pressure inward toward the center of funnel seal 722. At least a distal portion of cylindrical portion 732 seals around the medical device and provides support to the inserted medical device. Additionally, the drag from the insertion of the medical device through the distal portion of funnel portion 740, or second hole 744, will cause a proximal surface defining funnel portion 740 to roll inward and hug the shaft of the inserted medical device, providing additional surface area for sealing against the inserted medical device.

The medical device can be retracted from second hole 744 through funnel portion 740 and out of first hole 742 to remove the medical device from funnel seal 722. When the medical device is retracted, the material of funnel seal 722 and the shape of funnel portion 740 lower the force caused by retraction. Because funnel seal 722 is manufactured by molding funnel seal 722 first and subsequently creating second hole 744 in funnel seal 722 a separate step, funnel seal 722 recovers back to its original position to seal completely. Curved ribs 734 provide controlled return to the original position. As such, funnel seal 722 also provides an effective seal when no device is present within funnel seal 722.

When funnel seal 722 is positioned within a seal hub, such as seal hub 12, 112, or 212, the outer diameter of disc 728 of funnel seal 722 radially seals against the seal hub. The outer diameter of disc 728 forms a sealing surface that has an interference fit within the seal hub. As such, the outer diameter of disc 728 abuts and seals against the inner diameter of the seal hub, which provides a seal between the seal hub and funnel seal 722. The outer diameter of disc 728 of funnel seal 722 also interacts with the seal hub to provide retention within the seal hub. Further, disc 728 provides support for funnel portion 536 of funnel seal 722. Funnel seal 722 is made of a flexible material that lowers insertion and retraction pressure. Disc 728 provides radial structure to flexible funnel seal 722, allowing for axial and radial compression within the seal hub to prevent leak paths. Curved ribs 734 of funnel seal 722 allow for more controlled radial compression. Space within the seal hub is also available for annular portion 730 of funnel seal 722 to expand radially due to the smaller outer diameter of annular portion 730. As funnel seal 722 distorts, the counterforce of the insertion of the medical device is minimized, helping to retain funnel seal 722 within the seal hub.

The medical device stays at a center of funnel seal 722 as the medical device is being inserted due to funnel portion 740 and curved ribs 734 of funnel seal 722. As a result, the medical device is less likely to cause a tear in funnel seal 722, which can lead to hemostasis failure and/or detached particulate being introduced into the bloodstream. Because funnel portion 740 acts as a guide, the medical device is more likely to reach a center of distal end 726 of funnel seal 722, which is at second hole 744, and exit funnel seal 722 in the proper position, which is particularly beneficial for small devices. Because curved ribs 734 are curved, curved ribs 734 flex evenly to keep the inserted device centered and make funnel seal 722 less rigid, which is particularly beneficial for large diameter devices.

Funnel seal 722 provides a seal when nothing is inserted through funnel seal 722 and when devices of various diameters are inserted through funnel seal 722. Funnel seal 722 can accommodate a larger array of medical devices while reducing the risk of hemostasis loss. Funnel seal 722 seals on small diameter devices, such as a guidewire, and provides low insertion forces, which prevents damage when larger diameter devices, such as a dilator, are inserted through funnel seal 722. As such, funnel seal 722 prevents hemostasis loss with both small diameter and large diameter devices. Further, the reduced insertion force achieved by funnel seal 722 allows for easier insertion, retention, and retraction of a medical device and enables funnel seal 722 to withstand insertion and retraction of larger devices. As such, hemostasis loss is prevented during all portions of the medical procedure, including when nothing is inserted through funnel seal 722, when a small medical device is inserted through funnel seal 722, when a large medical device is inserted through funnel seal 722, and when a medical device is withdrawn from funnel seal 722. Because funnel seal 722 provides improved sealing upon insertion and retraction of a medical device, funnel seal 722 reduces the risk of fluid leakage. Funnel seal 722 is more successful at mitigating hemostasis loss for the patient and can be used in conjunction with a broader variety of medical devices and procedural configurations.

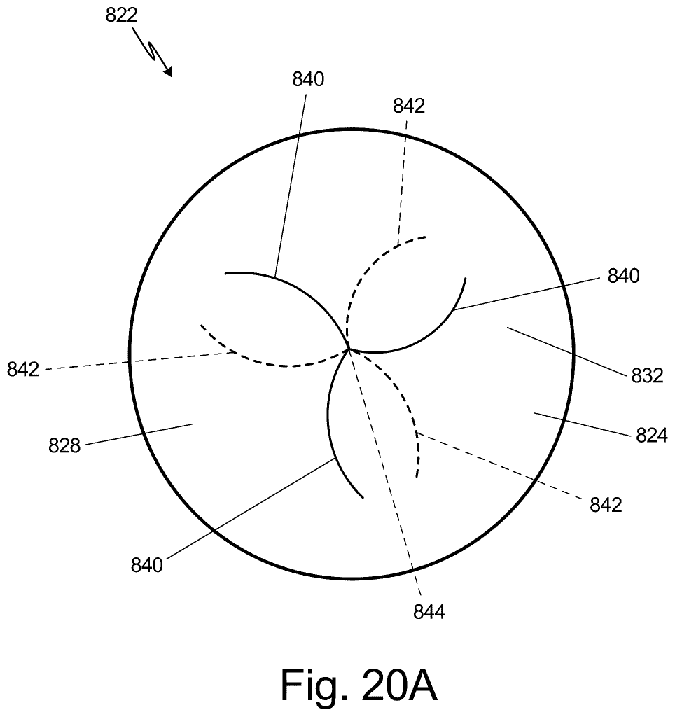

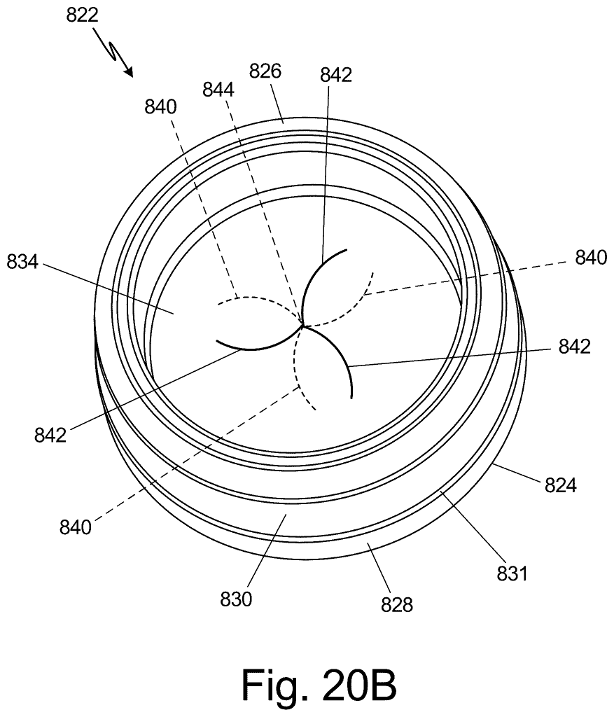

FIG. 20A is an end view of curved cross-plane seal 822 from proximal end 824 of curved cross-plane seal 822. FIG. 20B is a perspective view of curved cross-plane seal 822 from distal end 826 of curved cross-plane seal 822. FIGS. 20A and 20B will be discussed together.

Curved cross-plane seal 822 includes proximal end 824, distal end 826 (shown in FIG. 20B), disc 828, annular portion 830 (shown in FIG. 20B), and step 831 (shown in FIG. 20B). Disc 828 includes proximal face 832 (shown in FIG. 20A), distal face 834 (shown in FIG. 20B), first curved slits 840, second curved slits 842, and intersection 844.

Curved cross-plane seal 822 is made of a liquid injection silicone, or other similar material, that is cured following molding to stabilize the material. Curved cross-plane seal 822 has a U-shaped cross-sectional profile with a closed proximal end 824 and an open distal second end 326. Proximal end 824 is at a first end of curved cross-plane seal 822. Distal end 826 is at a second end of curved cross-plane seal 822 opposite proximal end 824. Disc 828 and annular portion 330 make up a body of cross-plane seal 322. Disc 828 is at proximal end 824 of curved cross-plane seal 822. Disc 828 is cylindrical, having a thickness. For example, disc 828 can have a thickness of ⅛ inch (0.3175 centimeter). Disc 828 has a greater outer diameter than an outer diameter of annular portion 830. Annular portion 830 is connected to and extends from disc 828 to distal end 826. Step 831 is formed by disc 828 having a greater outer diameter than the outer diameter of annular portion 830. Step 831 is formed where disc 828 is connected to annular portion 830 such that step 831 is between disc 828 and annular portion 830.

Disc 828 has proximal face 832 at the proximal side of disc 828 and distal face 834 at the distal side of disc 828. Proximal face 832 is at proximal end 824 of curved cross-plane seal 822. Disc 828 has a thickness between proximal face 832 and distal face 834. First curved slits 840 extend into proximal face 832 of disc 828, and second curved slits 842 extend into distal face 834 of disc 828.

First curved slits 840 are evenly spaced slits, each having a partial through-cut into proximal face 832 of disc 828. The through-cut of each of first curved slits 840 extends more than halfway, but not fully, through the thickness of disc 828. For example, first curved slits 840 may extend 3/32 inch (2.38 millimeters) deep through disc 828 having a material thickness of ⅛ inch (3.175 millimeters). In this example, first curved slits 840 may be ½ inch (12.7 millimeters) long within disc 828. First curved slits 840 can extend through between 50 percent and 100 percent of the thickness of disc 828, preferably at 75 percent of the thickness of disc 828. First curved slits 840 each have an end at a center of disc 828 and proximal face 832 and extend toward a periphery of disc 828. First curved slits 840 are spaced from each other and curved in a first direction, such as counterclockwise. In this example, curved cross-plane seal 822 includes three first curved slits 840. In alternate examples, curved cross-plane seal 822 may have any suitable number of first curved slits 840.

Second curved slits 842 are evenly spaced slits, each having a partial through-cut into distal face 834 of disc 828. The through-cut of each of second curved slits 842 extends more than halfway, but not fully, through the thickness of disc 828. For example, second curved slits 842 may extend 3/32 inch (2.38 millimeters) deep through disc 828 having a material thickness of ⅛ inch (3.175 millimeters). In this example, second curved slits 842 may be ½ inch (12.7 millimeters) long within disc 828. Second curved slits 842 can extend through between 50 percent and 100 percent of the thickness of disc 828, preferably at 75 percent of the thickness of disc 828. Second curved slits 842 each have an end at a center of disc 828 and distal face 834 and extend toward a periphery of disc 828. Second curved slits 842 are spaced from each other and curved in a second direction opposite the first direction, such as clockwise if first curved slits 840 are curved in a counterclockwise direction. If first curved slits 840 are curved in a clockwise direction, second curved slits 842 are curved in a counterclockwise direction. In this example, curved cross-plane seal 822 includes three second curved slits 842. In alternate examples, curved cross-plane seal 822 may have any suitable number of second curved slits 842. First curved slits 840 are offset from second curved sits 840. Because first curved slits 840 extend more than halfway through disc 828 from proximal face 832, second curved slits 842 extend more than halfway through disc 828 from second face 334, and first slit 340 and second slit 342 both start at a center of disc 828, first curved slits 840 and second curved slits 842 partially overlap to form intersection 844. As such, intersection 844 is within disc 828. Intersection 844 forms a hole, or pathway, through disc 828 between proximal face 832 of disc 828 and distal face 834 of disc 828 at a center of disc 828.

Curved cross-plane seal 822 may be used in place of cross-plane seal 22, cross-plane seal 122, or self-centering seal 222. As such, curved cross-plane seal 822 may have distal end 826 adjacent to and contacting duckbill seal 20, as described with respect to FIGS. 2A, 2B, and 3. Proximal end 824 of curved cross-plane seal 822 may be adjacent to and contacting ring 24, as described with respect to FIGS. 2A, 2B, and 3. Alternatively, curved cross-plane seal 822 may be used alone or with any combination of one or more seals. For example, curved cross-plane seal 822 may be the most proximal seal within a seal hub, such as seal hub 12, 112, or 212. The diameter and thickness of curved cross-plane seal 822, the depths of first curved slits 840 and second curved slits 842, and the number of first curved slits 840 and second curved slits 842 can vary to any suitable configuration to accommodate various medical devices.

Curved cross-plane seal 822 seals on medical devices of various sizes, such as a small diameter guidewire, a large diameter loader, or any other suitable device. As a medical device is inserted through curved cross-plane seal 822, the medical device exerts force on disc 828 of curved cross-plane seal 822. Intersection 844 of first curved slits 840 and second curved slits 842 is forced open to form a pathway through curved cross-plane seal 822 as curved cross-plane seal 822 elongates in response to the insertion force.

The medical device is extended through the hole at intersection 844. When the medical device is positioned within curved cross-plane seal 822, first curved slits 840 and second curved slits 842 seal around the medical device. First curved slits 840 and second curved slits 842 are positioned on opposite faces of disc 828, are offset, and are curved in opposing directions to provide a constant sealing effect. Further, first curved slits 840 and second curved slits 842 each extend less than fully through disc 828, or are partial through-cut slits, to provide a sealing region that is also able to retain the inserted medical device. The curvature of first curved slits 840 and second curved slits 842 and the offset on opposite faces of disc 822 of first curved slits 840 and second curved slits 842 mitigate drifting of inserted medical devices to help keep inserted medical devices in alignment with a center of curved cross-plane seal 822. The curvature of first curved slits 840 extending from proximal face 832 of disc 828 and the curvature of second curved slits 842 extending from distal face 834 of disc 828 further ensure that the inserted medical device remains centered. When a medical device, such as a loader with a large outer diameter, is inserted into curved cross-plane seal 822, annular portion 830 of cross-plane seal 822 flexes and expands radially.

The medical device can be retracted through the hole at intersection 844 to remove the medical device from curved cross-plane seal 822. When the medical device is retracted, the material of curved cross-plane seal 822, first curved slits 840, and second curved slits 842 lower the retraction force. First curved slits 840 and second curved slits 842 return to their original positions, and the hole at intersection 844 of disc 828 shrinks. Because first curved slits 840 and second curved slits 842 only partially overlap, curved cross-plane seal 822 provides an effective seal when no device is present within cross-plane seal 822. Additionally, curved cross-plane seal 822 is less likely to cave or fold in on itself, easing insertion and retraction of medical devices into and from cross-plane seal 822.

When curved cross-plane seal 822 is positioned within a seal hub, such as seal hub 12, 112, or 212, the outer diameter of disc 828 of curved cross-plane seal 822 radially seals against the seal hub. The outer diameter of disc 828 forms a sealing surface that has an interference fit within the seal hub. As such, the outer diameter of disc 828 abuts and seals against the inner diameter of the seal hub, which provides a seal between the seal hub and curved cross-plane seal 822. The outer diameter of disc 828 of curved cross-plane seal 822 also interacts with the seal hub to provide retention within the seal hub. Curved cross-plane seal 822 is made of a flexible material that lowers insertion and retraction pressure. Disc 828 provides radial structure to flexible curved cross-plane seal 822, allowing for axial and radial compression within the seal hub to prevent para-seal leak paths. Space within the seal hub is also available for annular portion 830 of curved cross-plane seal 822 to expand radially due to the smaller outer diameter of annular portion 830. As curved cross-plane seal 822 distorts, the counterforce of the insertion is minimized, helping to retain curved cross-plane seal 822 within the seal hub.

Curved cross-plane seal 822 provides a seal when nothing is inserted through curved cross-plane seal 822 and when devices of various diameters are inserted through curved cross-plane seal 822. Curved cross-plane seal 822 seals on small diameter devices, such as a guidewire, and provides low insertion forces, which prevents damage when larger diameter devices, such as a dilator, are inserted through curved cross-plane seal 822. As such, curved cross-plane seal 822 prevents hemostasis loss with both small diameter and large diameter devices. Further, the reduced insertion force achieved by curved cross-plane seal 822 allows for easier insertion, retention, and retraction of a medical device and enables curved cross-plane seal 822 to withstand insertion and retraction of larger devices. As such, hemostasis loss is prevented during all portions of the medical procedure, including when nothing is inserted through curved cross-plane seal 822, when a small medical device is inserted through curved cross-plane seal 822, when a large medical device is inserted through curved cross-plane seal 822, and when a medical device is withdrawn from curved cross-plane seal 822. Because curved cross-plane seal 822 provides improved sealing upon insertion and retraction of a medical device both coaxially and non-coaxially, curved cross-plane seal 822 reduces the risk of fluid leakage. Curved cross-plane seal 822 is more successful at mitigating hemostasis loss for the patient and can be used in conjunction with a broader variety of medical devices and procedural configurations.

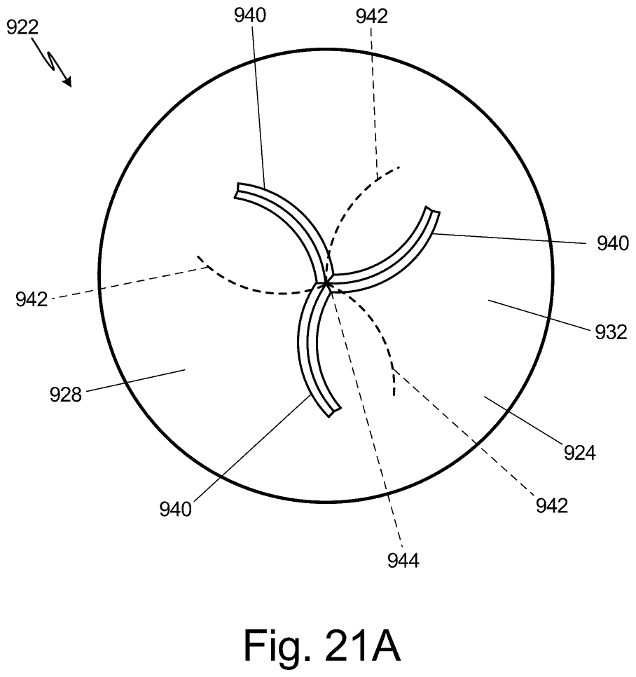

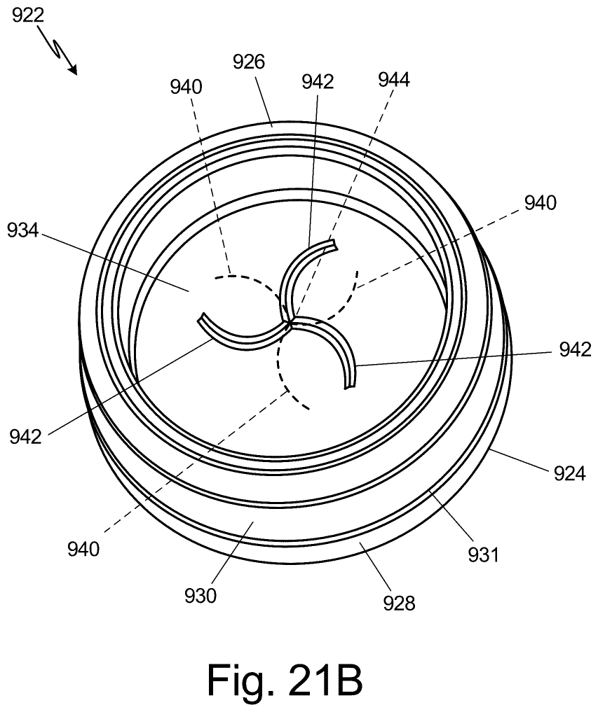

FIG. 21A is an end view of curved cross-plane seal 922 from proximal end 924 of curved cross-plane seal 922 showing beveled first curved slits 940 and beveled second curved slits 942. FIG. 21B is a perspective view of curved cross-plane seal 922 from distal end 926 of curved cross-plane seal 922. FIGS. 21A and 21B will be discussed together.

Curved cross-plane seal 922 includes proximal end 924, distal end 926 (shown in FIG. 21B), disc 928, annular portion 930 (shown in FIG. 21B), and step 931 (shown in FIG. 21B). Disc 928 includes proximal face 932 (shown in FIG. 21A), distal face 934 (shown in FIG. 21B), beveled first curved slits 940, beveled second curved slits 942, and intersection 944.

Curved cross-plane seal 922, shown in FIGS. 21A, 21B, and 21C, has generally the same structure and function as curved cross-plane seal 822, shown in and described with respect to FIGS. 20A and 20B. However, curved cross-plane seal 922 includes beveled first curved slits 940 and beveled second curved slits 942 instead of first curved slits 840 and second curved slits 842. Beveled first curved slits 940 and beveled second curved slits 942 have generally the same structure and function as first curved slits 840 and second curved slits 842 except that beveled first curved slits 940 and beveled second curved slits 942 are beveled. Edges of beveled first curved slits 940 and beveled second curved slits 942 are beveled as first curved slits 940 and second curved slits 942 extend inward from proximal face 932 and distal face 934, respectively.

Beveled first curved slits 940 and beveled second curved slits 942 create a muted funnel effect where beveled first curved slits 940 and beveled second curved slits 942 each form a small funnel. As a result, beveled first curved slits 940 and beveled second curved slits 942 help guide the medical device toward the center of curved cross-plane seal 922 during the insertion of the medical device, ensuring proper positioning of the inserted medical device.

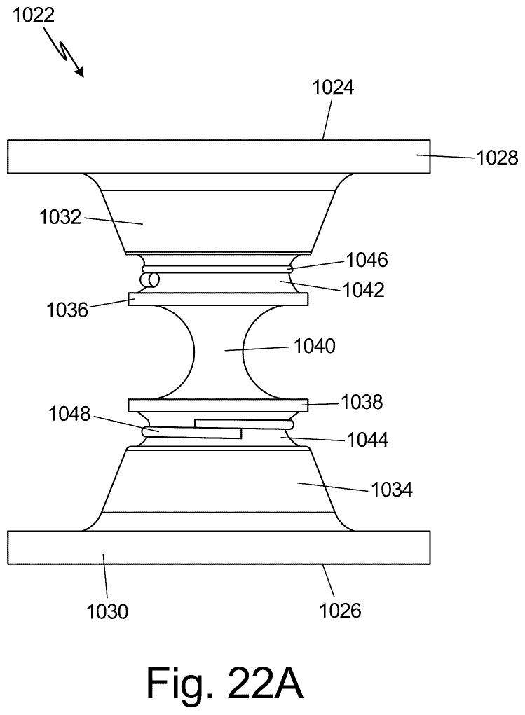

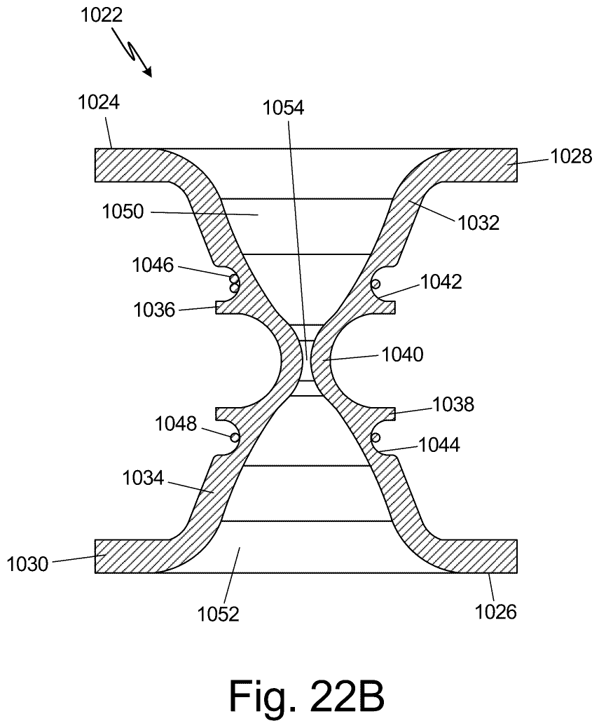

FIG. 22A is a side view of cinched seal 1022. FIG. 22B is a cross-sectional side view of cinched seal 1022. FIGS. 22A and 22B will be discussed together.

Cinched seal 1022 includes proximal end 1024, distal end 1026, proximal flange 1028, distal flange 1030, proximal conical portion 1032, distal conical portion 1034, proximal central flange 1036, distal central flange 1038, center portion 1040, proximal annular indent 1042, distal annular indent 1044, proximal coil 1046, distal coil 1048, proximal conical space 1050 (shown in FIG. 22B), distal conical space 1052 (shown in FIG. 22B), and hole 1054 (shown in FIG. 22B).

Cinched seal 1022 has proximal end 1024 at a first end of cinched seal 1022 and distal end 1026 at the other, second end of cinched seal 1022 opposite proximal end 1024. Proximal flange 1028 is a flange at proximal end 1024 of cinched seal 1022, a proximal end of proximal flange 1028 defining proximal end 1024. Distal flange 1030 is a flange at distal end 1026 of cinched seal 1022, a distal end of distal flange 1030 defining distal end 1026. Proximal conical portion 1032 is a conical-shaped portion that is connected to and extends away from proximal flange 1028. Distal conical portion 1034 is a conical-shaped portion that is connected to and extends away from distal flange 1030. Proximal central flange 1036 is an annular flange connected to a side of proximal conical portion 1032. Distal central flange 1038 is an annular flange connected to a side of distal conical portion 1034. Proximal flange 1028, proximal conical portion 1032, proximal central flange 1036, distal flange 1030, distal conical portion 1034, and distal central flange 1038 are made of a less elastic or semi-elastic, stiffer material, than the flexible, elastic material of center portion 1040. Center portion 1040 is connected to proximal conical portion 1032 and distal conical portion 1034 such that center portion 1040 is between proximal conical portion 1032 and distal conical portion 1034. Center portion 1040 is annular and has a C-shaped cross-section, as shown in FIG. 22B, such that an outer surface of center portion 1040 is concave. Ends of center portion 1040 are connected to proximal central flange 1036 and distal central flange 1038, respectively. Center portion 1040 of cinched seal 1022 is made of a flexible, elastic material.

Proximal annular indent 1042 is an annular indent in an outer surface of proximal conical portion 1032 adjacent proximal central flange 1036. Distal annular indent 1044 is an annular indent in an outer surface of distal conical portion 1034 adjacent distal central flange 1038. Proximal coil 1046 is a spring tempered coil, or c-ring, positioned on proximal conical portion 1032 adjacent center portion 1040 and in proximal annular indent 1042. Distal coil 1048 is a spring tempered coil, or c-ring, positioned on distal conical portion 1034 adjacent center portion 1040 and in distal annular indent 1044. As such, center portion 1040 is between proximal coil 1046 and distal coil 1048. Proximal coil 1046 and distal coil 1048 provide compression forces that are great enough to cinch center portion 1040 closed but low enough that push forces are not too great for the insertion of large diameter devices.

Proximal conical space 1050 is a conical space within proximal conical portion 1032, proximal conical space 1050 decreasing toward center portion 1040. Distal conical space 1052 is a conical space within distal conical portion 1034, distal conical space 1052 decreasing toward center portion 1040. Hole 1054 is an opening that extends through center portion 1040. Hole 1054 opens when a device is inserted through cinched seal 1022, easily when a small device is inserted and requiring additional force when a large device is inserted. Hole 1054 is fluidly connected to proximal conical space 1050 and distal conical space 1052, such that hole 1054 is between proximal conical space 1050 and distal conical space 1052.

Cinched seal 1022 may be used in place of cross-plane seal 22, cross-plane seal 122, or self-centering seal 222. As such, cinched seal 1022 may have distal end 1026 adjacent to and contacting duckbill seal 20, as described with respect to FIGS. 2A, 2B, and 3. Proximal end 1024 of cinched seal 1022 may be adjacent to and contacting ring 24, as described with respect to FIGS. 2A, 2B, and 3. Alternatively, cinched seal 1022 may be used alone or with any combination of one or more seals. For example, cinched seal 1022 may be the most proximal seal within a seal hub, such as seal hub 12, 112, or 212. The length and thickness of cinched seal 1022, the shape and curvature of proximal conical portion 1032 and distal conical portion 1034, the material of funnel seal 722, and the spring force of proximal coil 1046 and distal coil 1048 can vary to result in any suitable configuration to accommodate various medical devices.

Cinched seal 1022 seals on medical devices of various sizes, such as a small diameter guidewire, a large diameter loader, or any other suitable device. Proximal coil 1046 and distal coil 1048 together continuously provide active spring force on both sides of center portion 1040. When no device is inserted in cinched seal, force from proximal coil 1046 and distal coil 1048 cinch center portion 1040 closed. Proximal conical portion 1032 provides a visual target for insertion of a medical device through cinched seal 1022 when cinched seal 1022 is the most proximal seal. The medical device is extended through hole 1054. As the medical device enters proximal conical portion 1032, the medical device extends first through a portion of proximal conical space 1050 that has the largest diameter. The medical devices continues through proximal conical space 1050, proximal conical portion 1032 guiding the distal tip of the medical device to hole 1054 in center portion 1040.

Hole 1054 is forced open, center portion 1040 being forced outward in response to the insertion force of the medical device. Center portion 1040 is made of a material having elasticity, reducing the force required for the medical device to penetrate through hole 1054. Smaller diameter devices can easily pass through hole 1054 in center portion 1040. Larger diameter devices require more insertion force to pass through hole 1054 in center portion 1040, needing to counteract the spring force to further open cinched center portion 1040. Center portion 1040 bows out and increases the size of hole 1054. The distal end of the medical device subsequently enters distal conical space 1052 and is guided down the middle of cinched seal 1022 by distal conical portion 1034.

At least a portion of center portion 1040 seals around the medical device and provides support to the inserted medical device. Proximal coil 1046 and distal coil 1048 exert inward pressure, from spring force, on the medical device, strengthening the seal. Proximal annular indent 1042 and proximal central flange 1036 prevent proximal coil 1046 from migrating along cinched seal 1022. Distal annular indent 1044 and distal central flange 1038 prevent distal coil 1048 from migrating along cinched seal 1022. As such, proximal coil 1046 and distal coil 1048 remain in the same position with respect to center portion 1040. Proximal conical portion 1032 and distal conical portion 1034 are made of a stiffer material than center portion 1040 to provide additional support to cinched seal 1022, helping keep the medical device at the center of cinched seal 1022 during insertion.

The medical device can be retracted from distal conical space 1052 through center portion 1040 and out of proximal conical space 1050 to remove the medical device from cinched seal 1022. When the medical device is retracted, the spring force of distal coil 1048 and proximal coil 1046 causes center portion 1040 of cinched seal 1022 to quickly recover back to the original shape of center portion 1040, closing center portion 1040. Cinched seal 1022 quickly moves back to its original position to provide a seal when no device is present within cinched seal 1022.

When cinched seal 1022 is positioned within a seal hub, such as seal hub 12, 112, or 212, the outer diameters of proximal end 1024 and distal end 1026 of funnel seal 722 radially seal against the seal hub. The outer diameters of proximal flange 1028 and distal flange 1030 form sealing surfaces that have interference fits within the seal hub. As such, the outer diameters of proximal flange 1028 and distal flange 1030 abut and seal against the inner diameter of the seal hub, which provides a seal between the seal hub and cinched seal 1022. The outer diameters of proximal flange 1028 and distal flange 1030 of cinched seal 1022 also interact with the seal hub to provide retention within the seal hub. Proximal flange 1028 and distal flange 1030 provide radial structure to cinched seal 1022, allowing for axial and radial compression within the seal hub to prevent para-seal leak paths.

Proximal conical portion 1032 and distal conical portion 1034 act as guides for the insertion of the medical device, keeping the medical device at a center of cinched seal 1022. As a result, the medical device is less likely to cause a tear in cinched seal 1022, which can lead to hemostasis failure and/or detached particulate being introduced into the bloodstream. The medical device reaches center portion 1040 of cinched seal 1022 and exits cinched seal 1022 in the proper position, which is particularly beneficial for small devices. Because proximal coil 1046 and distal coil 1048 exert pressure to force center portion 1040 to close, cinched seal 1022 returns to its original position more quickly after a medical device is removed from cinched seal 1022. As such, cinched seal 1022 is capable of sealing more quickly, which is particularly beneficial for large diameter devices that force center portion 1040 outward to a greater extent.

Cinched seal 1022 provides a seal when nothing is inserted through cinched seal 1022 and when devices of various diameters are inserted through cinched seal 1022. Cinched seal 1022 seals on small diameter devices, such as a guidewire, and provides low insertion forces, which prevents damage when larger diameter devices, such as a dilator, are inserted through cinched seal 1022. As such, cinched seal 1022 prevents hemostasis loss with both small diameter and large diameter devices. Further, the reduced insertion force achieved by cinched seal 1022 allows for easier insertion, retention, and retraction of a medical device and enables cinched seal 1022 to withstand insertion and retraction of larger devices. As such, hemostasis loss is prevented during all portions of the medical procedure, including when nothing is inserted through cinched seal 1022, when a small medical device is inserted through cinched seal 1022, when a large medical device is inserted through cinched seal 1022, and when a medical device is withdrawn from cinched seal 1022. Because cinched seal 1022 provides improved sealing upon insertion and retraction of a medical device, cinched seal 1022 reduces the risk of fluid leakage. Cinched seal 1022 is more successful at mitigating hemostasis loss for the patient and can be used in conjunction with a broader variety of medical devices and procedural configurations.

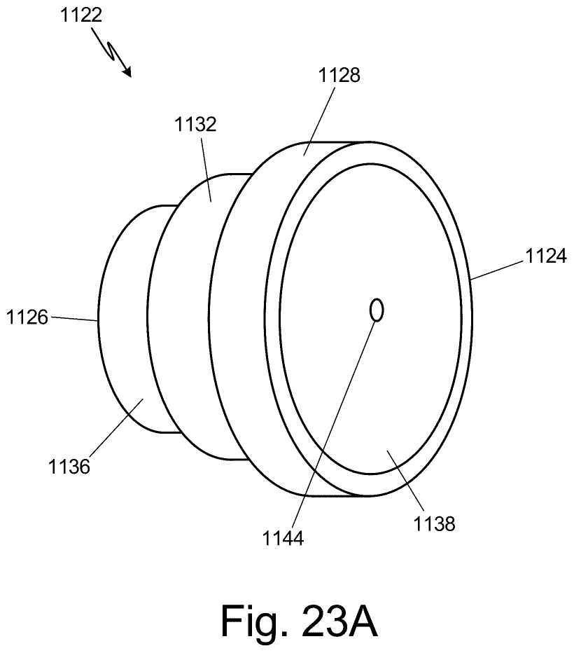

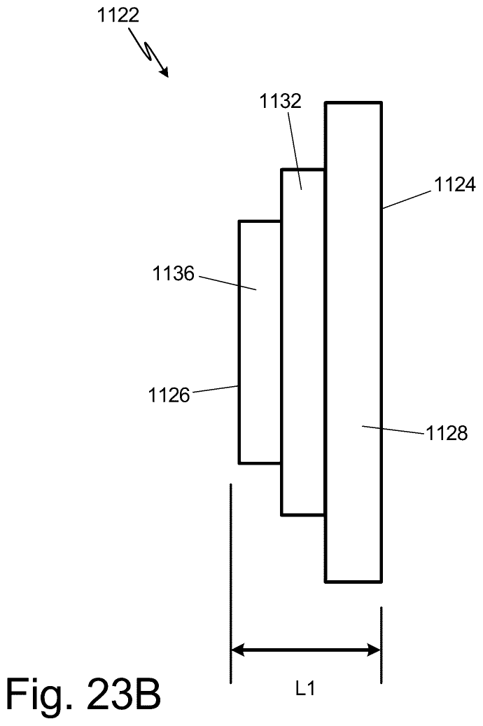

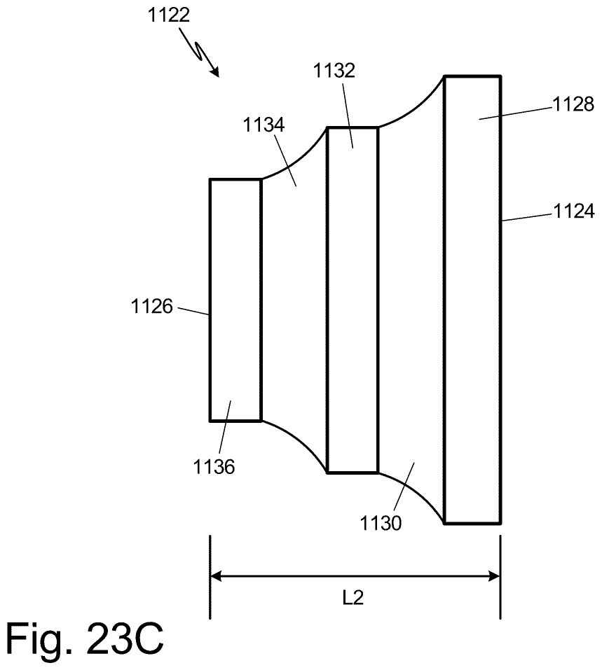

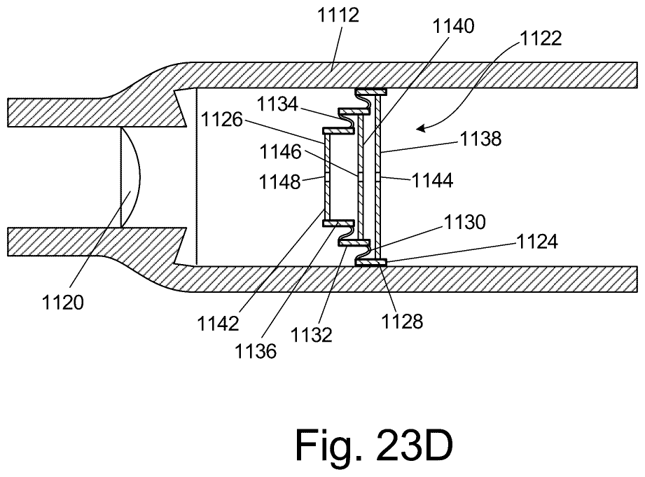

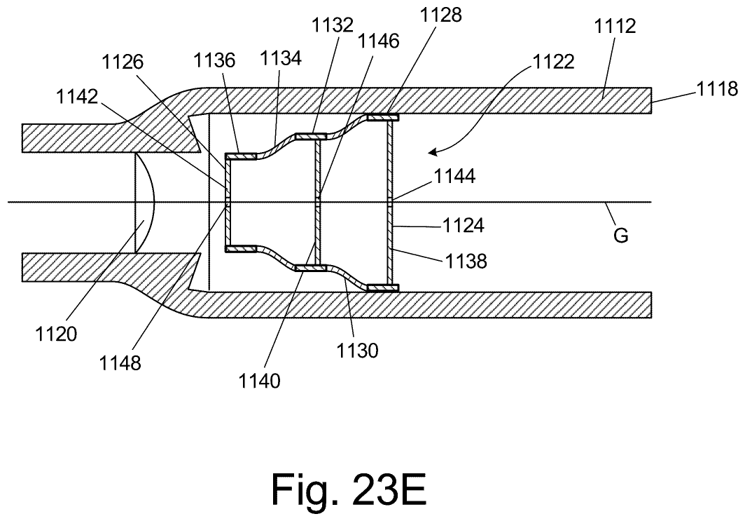

FIG. 23A is an isometric view of collapsible seal 1122 in a collapsed position. FIG. 23B is a side view of collapsible seal 1122 in the collapsed position. FIG. 23C is a side view of collapsible seal 1122 in an expanded position. FIG. 23D is a cross-sectional side view of seal hub 1112 with collapsible seal 1122 when no device is inserted into seal hub 1112. FIG. 23E is a cross-sectional side view of seal hub 1112 with collapsible seal 1122 when a device is inserted into seal hub 1112. FIGS. 23A, 23B, 23C, 23D, and 23E will be discussed together.

Collapsible seal 1122 includes proximal end 1124, distal end 1126, first ring 1128, first membrane 1130 (shown in FIGS. 23C, 23D, and 23E), second ring 1132, second membrane 1134 (shown in FIGS. 23C, 23D, and 23E), third ring 1136, first wall 1138 (shown in FIGS. 23A, 23D, and 23E), second wall 1140 (shown in FIG. 23D), third wall 1142 (shown in FIG. 23E), first hole 1144 (shown in FIGS. 23A, 23D, and 23E), second hole 1146 (shown in FIGS. 23D and 23E), and third hole 1148 (shown in FIGS. 23D and 23E). Also shown in FIGS. 23D and 23E are seal hub 1112 and duckbill seal 1120.

Collapsible seal 1122 has proximal end 1124 at a first end of collapsible seal 1122 and distal end 1126 at the other, second end of collapsible seal 1122 opposite proximal end 1124. First ring 1128 is a proximal ring of collapsible seal 1122 and defines proximal end 1124 of collapsible seal 1122. First ring 1128 has a diameter such that first ring 1128 seals against an inner diameter of seal hub 1112, as seen in FIGS. 23D and 23E. A distal end of first ring 1128 is connected to a proximal end of first membrane 1130. A distal end of first membrane 1130 is connected to a proximal end of second ring 1132 such that first membrane 1130 is between first ring 1128 and second ring 1132, connecting first ring 1128 to second ring 1132. Second ring 1132 has a smaller diameter than the diameter of first ring 1128. A distal end of second ring 1132 is connected to a proximal end of second membrane 1134. A distal end of second membrane 1134 is connected to a proximal end of third ring 1136 such that the second membrane 1134 is between second ring 1132 and third ring 1136, connecting second ring 1132 to third ring 1136. Third ring 1136 is a distal ring of collapsible seal 1122 and defines distal end 1126 of collapsible seal 1122. Third ring 1136 has a smaller diameter than the diameter of second ring 1132. In alternate examples, collapsible seal 1122 does not include third ring 1136 or second membrane 1134.

First membrane 1130 and second membrane 1134 are made of a material that is thin and/or has a low durometer such that first membrane 1130 and second membrane 1134 are flexible or elastic. First ring 1128, second ring 1132, and third ring 1136 are made of a material that is thicker and/or has a higher durometer than the material of first membrane 1130 and second membrane 1134 such that the first ring 1128, second ring 1132, and third ring 1136 are less flexible or elastic. First ring 1128, second ring 1132, and third ring 1136 are rigid. In alternate examples, collapsible seal 1122 may have more than three rings and more than two membranes.

First wall 1138 is connected to an inner circumference of first ring 1128 and extends across an entire area of first ring 1128. Second wall 1140 is connected to an inner circumference of second ring 1132 and extends across an entire area of second ring 1132. Third wall 1142 is connected to an inner circumference of third ring 1136 and extends across an entire area of third ring 1136. First wall 1138, second wall 1140, and third wall 1142 are made of a material that is thinner and/or low has a lower durometer than first ring 1128, second ring 1132, and third ring 1136 such that first wall 1138, second wall 1140, and third wall 1142 are flexible or elastic. First hole 1144 is an opening that extends through a center of first wall 1138. Second hole 1146 is an opening that extends through a center of second wall 1140 such that second hole 1146 is in alignment with first hole 1144. Third hole 1148 is an opening that extends through a center of third wall 1142 such that third hole 1148 is in alignment with second hole 1146 and first hole 1144. In this example, first hole 1144, second hole 1146, and third hole 1148 are circular spaces. In alternate examples, first hole 1144, second hole 1146, and third hole 1148 may be spaces of any suitable shape, cross-slits, or any other suitable opening. In further alternate examples, first hole 1144, second hole 1146, and third hole 1148 may be off-center through first wall 1138, second wall 1140, and third wall 1142, respectively.

Collapsible seal 1122 may be used along with or in place of cross-plane seal 22, cross-plane seal 122, or self-centering seal 222. Collapsible seal 1122 may have distal end 1126 adjacent to, and potentially contacting, duckbill seal 1120, as shown in FIGS. 23D and 23E, and as described with respect to duckbill seal 20, shown in FIGS. 2A, 2B, and 3. Proximal end 1124 of collapsible seal 1122 may be adjacent to and contacting a distal end of cross-plane seal 22, cross-plane seal 122, or self-centering seal 222, as described with respect to FIGS. 2A, 2B, and 3. Alternatively, collapsible seal 1122 may be used alone or with any combination of one or more seals. For example, collapsible seal 1122 may be the most proximal seal within seal hub 1112. The length and thickness of collapsible seal 1122, the shape and size of first hole 1144, second hole 1146, and third hole 1148, the length and durometer of the material of first membrane 1130 and second membrane 1134, and the number of rings and membranes of collapsible seal 1122 can vary to result in any suitable configuration to accommodate various medical devices.

Collapsible seal 1122 seals on medical devices of various sizes, such as a small diameter guidewire, a large diameter loader, or any other suitable device. Collapsible seal 1122 is initially in a collapsed position, as shown in FIGS. 23B and 23D. In a fully collapsed position, collapsible seal 1122 has length L1, as shown in FIG. 23B. Third ring 1136 is partially nested inside second ring 1132, which is partially nested inside first ring 1128. In the collapsed position, first membrane 1130 is folded into an S-shaped fold between first ring 1128 and second ring 1132, and second membrane 1134 is folded into an S-shaped fold between second ring 1132 and third ring 1136.

The medical device is extended through first hole 1144 in first wall 1138, expanding first hole 1144 to fit through first hole 1144. The medical device is subsequently extended through second hole 1146 in second wall 1140, expanding second hole 1146 to fit through second hole 1146. The medical device stretches collapsible seal 1122 to expand collapsible seal 1122 via insertion force. As the medical device is extended through second hole 1146, the insertion force pushes second ring 1132 distally. As second ring 1132 is moved distally, first membrane 1130 unfolds. The medical device is then extended through third hole 1148 in third wall 1142, expanding third hole 1148. The insertion force pushes third ring 1136 distally. As third ring 1136 is moved distally, second membrane 1134 unfolds. First hole 114, second hole 1146, and third hole 1148 guide the medical device down a center of collapsible seal 1122, seal around the inserted medical device, and provide support to the inserted medical device.

First membrane 1130 and second membrane 1134 will unfold to the extent of the respective insertion forces. When a smaller medical device, such as a guidewire, is inserted, less insertion force is generated and collapsible seal 1122 is extended to a lesser extent. As such, second ring 1132 and third ring 1136 move distally to a lesser extent, and first membrane 1130 and second membrane 1134 unfold to a lesser extent. When a larger medical device, such as an insertion device, is inserted, more insertion force is generated and collapsible seal 1122 is extended to a greater extent. As such, second ring 1132 and third ring 1136 move distally to a greater extent or to the greatest extent, and first membrane 1130 and second membrane 1134 unfold to a greater extent or unfold completely, as shown in FIGS. 23C and 23E. In an expanded position, collapsible seal 1122 has length L2, as shown in FIG. 23C. Length L2 is greater than length L1.

Because first membrane 1130 and second membrane 1134 are elastic, first membrane 1130 and second membrane 1134 can stretch beyond a fully unfolded position to counteract a large insertion force. Collapsible seal 1122 starts to expand as a medical device is inserted, and the expansion of collapsible seal 1122 reduces the force required for the medical device to penetrate through first hole 1144, second hole 1146, and third hole 1148 of collapsible seal 1122. First wall 1138, second wall 1140, and third wall 1142 all provide redundant sealing. First ring 1128, second ring 1132, and third ring 1136 are made of a stiffer material than first membrane 1130 and second membrane 1134 to provide additional support to collapsible seal 1122, helping to keep the medical device at the center of collapsible seal 1122 during insertion of the medical device.

The medical device is retracted from third hole 1148, second hole 1146, and first hole 1144, through a center of collapsible seal 112, to remove the medical device from collapsible seal 1122. When the medical device is retracted from collapsible seal 1122, frictional force from retracting the medical device causes collapsible seal 1122 to collapse and begin to recover partially or fully back to a collapsed state. The larger the medical device, the more likely collapsible seal 1122 will revert back to a fully collapsed state upon retraction of the medical device. Collapsible seal 1122 recovers back to a collapsed state to provide a seal when no device is present within collapsible seal 1122.

When collapsible seal 1122 is positioned within seal hub, such as seal hub 12, 112, 212, or 1112, collapsible seal 1122 is in a collapsed state and the outer diameter of first ring 1128 radially seals against the seal hub. The outer diameter of first ring 1128 forms a sealing surface that has an interference fit within the seal hub. As such, the outer diameter of first ring 1128 abuts and seals against the inner diameter of the seal hub, which provides a seal between the seal hub and collapsible seal 1122. The outer diameter of first ring 1128 of cinched seal 1022 also interacts with the seal hub to provide retention within the seal hub. First ring 1128, second ring 1132, and third ring 1136 provide radial structure to collapsible seal 1122. As collapsible seal 1122 expands and collapses, the counterforce of insertion and retraction is minimized, helping to retain collapsible seal 1122 within seal hub 1112.

The medical device remains centered within collapsible seal 1122 as the medical device is being inserted. As a result, the medical device is less likely to cause a tear in collapsible seal 1122, which can lead to hemostasis failure and/or detached particulate being introduced into the bloodstream. Collapsible seal 1122 is a single seal that functions as multiple seals, due to first ring 1128 with first wall 1138, second ring 1132 with second wall 1140, and third ring 1136 with third wall 1142, but is a single component that occupies less area within seal hub 1112. By utilizing a single collapsible seal 1122 to maintain hemostasis, the number of necessary components is reduced and space efficiency is achieved within seal hub 1112.

Collapsible seal 1122 provides a seal when nothing is inserted through collapsible seal 1122 and when devices of various diameters are inserted through collapsible seal 1122. Collapsible seal 1122 can accommodate a larger array of medical devices while reducing the risk of hemostasis loss. Collapsible seal 1122 seals on small diameter devices, such as a guidewire, and provides low insertion forces, which prevents damage when larger diameter devices, such as a dilator, are inserted through collapsible seal 1122. As such, collapsible seal 1122 prevents hemostasis loss with both small diameter and large diameter devices. Further, the reduced insertion force achieved by collapsible seal 1122 allows for easier insertion, retention, and retraction of a medical device and enables collapsible seal 1122 to withstand insertion and retraction of larger devices. As such, hemostasis loss is prevented during all portions of the medical procedure, including when nothing is inserted through collapsible seal 1122, when a small medical device is inserted through collapsible seal 1122, when a large medical device is inserted through collapsible seal 1122, and when a medical device is withdrawn from collapsible seal 1122. Because collapsible seal 1122 provides redundancy for improved sealing upon insertion and retraction of a medical device, collapsible seal 1122 reduces the risk of fluid leakage. Collapsible seal 1122 is more successful at mitigating hemostasis loss for the patient and can be used in conjunction with a broader variety of medical devices and procedural configurations.

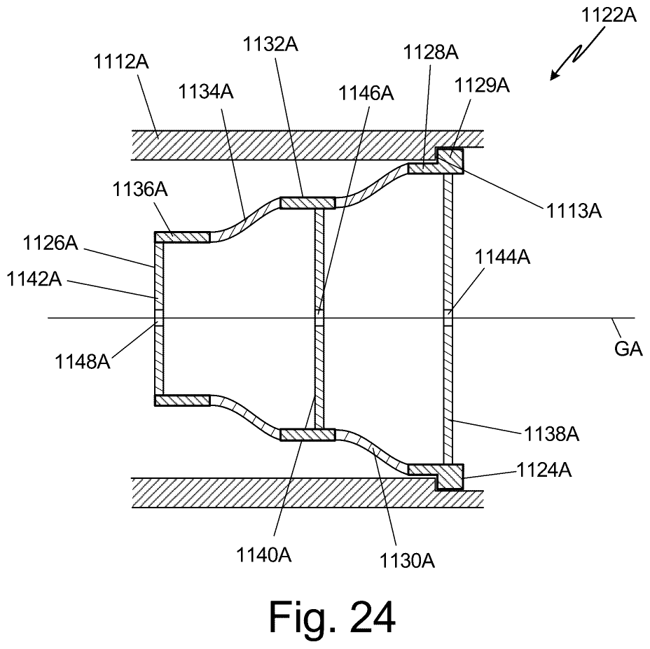

FIG. 24 is a cross-sectional side view of seal hub 1112A with collapsible seal 1122A when a device is inserted into seal hub 1112A.

Collapsible seal 1122A includes proximal end 1124A, distal end 1126A, first ring 1128A, first membrane 1130A, second ring 1132A, second membrane 1134A, third ring 1136A, first wall 1138A, second wall 1140A, third wall 1142A, first hole 1144A, second hole 1146A, third hole 1148A, and flange 1150A. Also shown in FIG. 24 is seal hub 1112A, which has step 1113A.

Collapsible seal 1122A, shown in FIG. 24, has generally the same structure and function as collapsible seal 1122, shown in and described with respect to FIGS. 23A-23E. However, collapsible seal 1122A includes flange 1150A and seal hub 1112A has step 1113A. Flange 1150A is connected to an outer surface of first ring 1128A. Flange 1150A may be ring-like, thus, having a larger outer diameter than an outer diameter of first ring 112A. When collapsible seal 1122A is positioned within seal hub 1112A, flange 1150A contacts step 1113A in seal hub 1112A. Step 1113A is formed by an interior surface of seal hub 1112A, where a proximal portion of seal hub 1112A has a larger inner diameter.

Flange 1150A of collapsible seal 1122A contacts, or rests on, step 1113A such that flange 1150A cannot move distally beyond step 1113A. As a result, collapsible seal 1122A becomes locked so that collapsible seal 1122A cannot move any further distally within seal hub 1112A. Thus, when guidewire GA is inserted through seal hub 1112A and collapsible seal 1122A, collapsible seal 1122A is held in position within seal hub 1112A and expands. Step 1113A and flange 1150A prevent distal migration of collapsible seal 1122A, allowing collapsible seal 1122A to withstand larger insertion forces.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Discussion of Detailed Examples

The following are non-exclusive descriptions of possible examples of the present invention.

A cross-plane seal having a distal end and a proximal end includes a disc at the distal end of the cross-plane seal, the disc comprising: a distal face at a distal side of the disc; a proximal face at a proximal side of the disc; a plurality of cut outs in the distal face of the disc forming a raised X-shape on the distal face of the disc; a first slit having a partial through-cut into the distal face of the disc and aligned with a first leg of the raised X-shape, the through-cut extending more than halfway through the thickness of the disc; a second slit having a partial through-cut into the proximal face of the disc and aligned with a second leg of the raised X-shape, the through-cut extending more than halfway through the thickness of the disc, wherein the first slit is perpendicular to the second slit; and an intersection formed by the first slit and the second slit; and an annular portion connected to and extending proximally away from the disc to the proximal end of the cross-plane seal.

The cross-plane seal of the preceding paragraph can optionally include, additionally and/or alternatively, any one or more of the following features, configurations and/or additional components:

The disc has a greater outer diameter than an outer diameter of the annular portion.

A step is formed between the disc and the annular portion.

An outer diameter of the disc forms a sealing surface that is configured to have an interference fit within a seal hub.

A cross-sectional profile of the cross-plane seal is U-shaped.

The cross-plane seal is made of a biocompatible polymer having a durometer of about 20 A and an elongation percentage of 900 percent to 1500 percent.

A seal stack comprises the cross-plane seal, and the seal stack is configured to fit within a seal hub of a guide sheath assembly.

The seal stack further comprises a duck bill seal, a ring, a disc seal, and a cap, the cross-plane seal being adjacent the duck bill seal and the ring.

The seal stack further comprises a cross-slit seal, a ring, a disc seal, and a cap, the cross-plane seal being adjacent the cross-slit seal and the ring.

The first slit and the second slit each extend through between 50 percent and 100 percent of the thickness of the disc.

The cut outs are wedge-shaped.

The cross-plane seal is sterilized.

A seal stack for a seal hub of a guide sheath assembly includes a cross-plane seal having a distal end and a proximal end and comprising: a disc at the distal end of the cross-plane seal, the disc comprising: a distal face at a distal side of the disc; a proximal face at a proximal side of the disc; a plurality of cut outs in the distal face of the disc forming a raised X-shape on the distal face of the disc; a first slit having a partial through-cut into the distal face of the disc and aligned with a first leg of the raised X-shape, the through-cut extending more than halfway through the thickness of the disc; a second slit having a partial through-cut into the proximal face of the disc and aligned with a second leg of the raised X-shape, the through-cut extending more than halfway through the thickness of the disc; and an intersection formed by the first slit and the second slit; and an annular portion connected to and extending proximally away from the disc to the proximal end of the cross-plane seal; a ring adjacent the cross-plane seal; a disc seal adjacent the ring; and a cap adjacent the disc seal.

The seal stack of the preceding paragraph can optionally include, additionally and/or alternatively, any one or more of the following features, configurations and/or additional components:

A duckbill seal adjacent the cross-plane seal.

A cross-slit seal adjacent the cross-plane seal.

The disc of the cross-plane seal has a greater outer diameter than an outer diameter of the annular portion of the cross-plane seal.

A step is formed between the disc of the cross-plane seal and the annular portion of the cross-plane seal.

An outer diameter of the disc of the cross-plane seal forms a sealing surface that is configured to have an interference fit within a seal hub.

A cross-sectional profile of the cross-plane seal is U-shaped.

The cross-plane seal is made of a biocompatible polymer having a durometer of about 20 A and an elongation percentage of 900 percent to 1500 percent.

The seal stack is configured to fit within the seal hub of a guide sheath assembly.

The first slit is perpendicular to the second slit.

The first slit and the second slit each extend through between 50 percent and 100 percent of the thickness of the disc.

The cut outs of the cross-plane seal are wedge-shaped.

The seal stack is sterilized.

The ring is positioned within the cross-plane seal.

The disc of the cross-plane seal is between the duckbill seal and the ring.

A self-centering seal includes a distal end and a proximal end. The self-centering seal further includes a disc including a plurality of raised curves extending from the distal end of the self-centering seal to the proximal end of the self-centering seal. The self-centering seal further includes a central planar region connected to an inner diameter of the disc, and a hole extending through the central planar region.

The self-centering seal of the preceding paragraph can optionally include, additionally and/or alternatively, any one or more of the following features, configurations and/or additional components:

The plurality of raised curves includes a flattened region between valleys on a distal face of the disc.

The disc has a rigid portion around an outer edge of the disc.

The disc includes a distal face extending from the distal end of the self-centering seal, and a proximal face extending from the proximal end of the self-centering seal.

The plurality of raised curves have different lengths.

An outer diameter of the disc of the self-centering seal forms a sealing surface that is configured to have an interference fit within a seal hub.

The self-centering seal is made of a biocompatible polymer having a durometer of about 40 A and an elongation percentage of 500 percent to 1200 percent.

The plurality of raised curves have a height between 150 percent and 1300 percent a thickness of the self-centering seal.

The self-centering seal is sterilized.

The self-centering seal is part of a seal stack, and the seal stack is configured to fit within a seal hub of a guide sheath assembly.

The seal stack includes a duckbill seal between a distal end and a proximal end of the seal hub, a ring, a cap, and a disc seal adjacent to the ring and the cap, wherein the self-centering seal is adjacent to the duckbill seal and the ring.

The ring is configured to fit between the duckbill seal and the self-centering seal.

A seal stack for a seal hub of a guide sheath assembly, the seal stack comprising a self-centering seal having a distal end and a proximal end, the self-centering seal comprising a disc comprising a plurality of raised curves extending axially from the distal end of the self-centering seal to the proximal end of the self-centering seal. The self-centering seal further includes a central planar region connected to an inner diameter of the disc and a hole extending through the central planar region. The seal stack further includes a ring adjacent to the self-centering seal, a disc seal adjacent to the ring, and a cap adjacent to the disc seal.

The seal stack of the preceding paragraph can optionally include, additionally and/or alternatively, any one or more of the following features, configurations and/or additional components:

A duckbill seal is adjacent to the self-centering seal.

A rigid portion around an outer edge of the disc.

The plurality of raised curves have different lengths.

An outer diameter of the disc of the self-centering seal forms a sealing surface that is configured to have an interference fit within the seal hub.

The self-centering seal is made of a biocompatible polymer having a durometer of about 40 A and an elongation percentage of 500 percent to 1200 percent.

The seal stack is configured to fit within the seal hub of the guide sheath assembly.

The seal stack is sterilized.

A cross-plane seal having a first end and a second end includes a disc at the first end of the cross-plane seal, the disc comprising: a first face at a first side of the disc; a second face at a second side of the disc; a star-shaped cut out having a star-shaped recess in the first face; a first slit having a partial through-cut into the first face of the disc and aligned with arms of the star-shaped cut out, the through-cut extending more than halfway through a thickness of the disc; a second slit having a partial through-cut into the second face of the disc and aligned with arms of the star-shaped cut out, the through-cut extending more than halfway through the thickness of the disc, wherein the first slit is perpendicular to the second slit; and an intersection formed by the first slit and the second slit; and an annular portion connected to and extending away from the disc.

The cross-plane seal of the preceding paragraph can optionally include, additionally and/or alternatively, any one or more of the following features, configurations and/or additional components:

The first end of the cross-plane seal is a distal end of the cross-plane seal.

The second end of the cross-plane seal is a distal end of the cross-plane seal.

The first face of the disc is adjacent the annular portion, the star-shaped cut out being adjacent the annular portion.

The second face of the disc is adjacent the annular portion.

The star-shaped cut out comprises a central region with eight rectangular arms evenly spaced around and extending from the central region, wherein the eight rectangular arms comprises the arms of the star-shaped cut out with which the first slit is aligned and the arms of the star-shaped cut out with which the second slit is aligned.

A funnel seal includes a proximal end, a distal end opposite the proximal end, a disc at the proximal end of the funnel seal, a proximal face of the disc forming a proximal face of the funnel seal, an annular portion connected to the disc, and a funnel portion forming a funnel-shaped opening that extends from the proximal end to the distal end of the funnel seal.

The funnel seal of the preceding paragraph can optionally include, additionally and/or alternatively, any one or more of the following features, configurations and/or additional components:

The annular portion is at the distal end of the funnel seal, a distal face of the annular portion forming a distal face of the funnel seal, and the funnel portion extends through the disc and the annular portion.

A first hole that forms a proximal end of the funnel portion and a second hole that forms a distal end of the funnel portion. The first hole has a diameter that is larger than a diameter of the second hole.

An outer diameter of the disc is greater than an outer diameter of the annular portion.

A ring portion connected to the annular portion such that the annular portion is between the disc and the ring portion, wherein the ring portion forms the distal end of the funnel seal.

A cylindrical portion extending from a central portion of the disc, the central portion being spaced from and concentric with annular portion and a plurality of curved ribs extending from an outer surface of the cylindrical portion to an inner surface of the annular portion.

A cross-plane seal having a proximal end and a distal end includes a disc at the proximal end of the cross-plane seal, the disc comprising: a proximal face at a proximal side of the disc; a distal face at a distal side of the disc; a plurality of first curved slits, each having a partial through-cut into the proximal face of the disc, the through-cut extending more than halfway through a thickness of the disc; a plurality of second curved slits, each having a partial through-cut into the proximal face of the disc, the through-cut extending more than halfway through the thickness of the disc, wherein the first plurality of curved slits are offset from the second plurality of curved slits; and an intersection formed by the first plurality of curved slits and the second plurality of curved slits; and an annular portion connected to and extending away from the disc.

The cross-plane seal of the preceding paragraph can optionally include, additionally and/or alternatively, any one or more of the following features, configurations and/or additional components:

The plurality of first curved slits comprises three first curved slits, and the plurality of second curved slits comprises three second curved slits.

The plurality of first curved slits are spaced from each other, and the plurality of second curved slits are spaced from each other.

The plurality of first curved slits are curved in a first direction, and the plurality of second curved slits are curved in a second direction opposite the first direction.

The plurality of first curved slits each have an end at a center of the disc and the plurality of second curved slits each have an end at a center of the disc.

A cinched seal having a proximal end and a distal end includes a proximal flange at the proximal end of the cinched seal; a distal flange at the distal end of the cinched seal; a proximal conical portion connected to and extending from the proximal flange; a proximal conical space within the proximal conical portion; a distal conical portion connected to and extending from the distal flange; a distal conical space within the distal conical portion; a center portion connected to the proximal conical portion and the distal conical portion, the center portion having a C-shaped cross-section; a hole extending through the center portion and fluidly connected to the proximal conical space and the distal conical space; a proximal coil positioned on the proximal conical portion adjacent the center portion; and a distal coil positioned on the distal conical portion adjacent the center portion.

The cinched seal of the preceding paragraph can optionally include, additionally and/or alternatively, any one or more of the following features, configurations and/or additional components:

A proximal central flange connected to a side of the proximal conical portion, and a distal central flange connected to a side of the distal conical portion.

A proximal annular indent in an outer surface of the proximal conical portion adjacent the proximal central flange and a distal annular indent in an outer surface of the distal conical portion adjacent the distal central flange, the proximal coil being positioned in the proximal annular indent and the distal coil being positioned in the distal annular indent.

The center portion is made of an elastic material and the proximal flange, the distal flange, the proximal conical portion, and the distal conical portion are made of a material that is less elastic than the elastic material of the center portion.

A collapsible seal having a proximal end and a distal end includes a first ring defining the proximal end of the collapsible seal; a first membrane connected to the first ring; a second ring connected to the first membrane such that the first membrane is between the first ring and the second ring; a first wall extending across the first ring; a second wall extending across the second ring; a first hole extending through a center of the first wall; and a second hole extending through a center of the second wall.

The collapsible seal of the preceding paragraph can optionally include, additionally and/or alternatively, any one or more of the following features, configurations and/or additional components:

The first membrane is made of a material having a lower durometer than a material of the first ring and the second ring.

The second ring has a diameter that is smaller than a diameter of the first ring.

A second membrane connected to the second ring; a third ring connected to the second membrane such that the second membrane is between the second ring and the third ring; a third wall extending across the third ring; and a third hole extending through a center of the third wall.

The third ring has a smaller diameter than the diameter of the second ring.

While the invention has been described with reference to an exemplary example(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular example(s) disclosed, but that the invention will include all examples falling within the scope of the appended claims.

1. A cinched seal having a proximal end and a distal end, the cinched seal comprising:
a proximal flange at the proximal end of the cinched seal;
a distal flange at the distal end of the cinched seal;
a proximal conical portion connected to and extending from the proximal flange;
a proximal conical space within the proximal conical portion; a distal conical portion connected to and extending from the distal flange;
a distal conical space within the distal conical portion;
a center portion connected to the proximal conical portion and the distal conical portion, the center portion having a C-shaped cross-section;
a hole extending through the center portion and fluidly connected to the proximal conical space and the distal conical space;
a proximal coil positioned on the proximal conical portion adjacent the center portion; and
a distal coil positioned on the distal conical portion adjacent the center portion.

2. The cinched seal of claim 1, wherein the hole in the center portion is movable between a closed position and an open position,
wherein the hole is movable toward the open position by an outwardly directed radial force against a central lumen of the hole.

3. The cinched seal of claim 1, wherein the proximal coil and the distal coil provide a compressive force on the center portion directing the hole toward a closed position.

4. The cinched seal of claim 1, further comprising a proximal central flange connected to a side of the proximal conical portion, and a distal central flange connected to a side of the distal conical portion.

5. The cinched seal of claim 4, further including a proximal annular indent in an outer surface of the proximal conical portion adjacent the proximal central flange and a distal annular indent in an outer surface of the distal conical portion adjacent the distal central flange.

6. The cinched seal of claim 5, wherein the proximal coil is positioned in the proximal annular indent and the distal coil is positioned in the distal annular indent such that the center portion located in a longitudinal direction along the cinched seal between the proximal coil and the distal coil,
wherein the proximal coil and the distal coil provide a compressive force on the center portion directing the hole toward a closed position.

7. The cinched seal of claim 1, wherein the C-shaped cross-section of the center portion is taken in a plane extending along a longitudinal axis of the cinched seal,
wherein an outer surface of the center portion is concave.

8. The cinched seal of claim 1, wherein a width of the proximal conical space decreases from the proximal end toward the center portion and a width of the distal conical space decreases from the distal end toward the center portion.

9. A seal hub assembly for a guide sheath, the seal hub assembly comprising:
a seal hub;
a seal stack received within the seal hub, the seal stack comprising:
a distal seal located within the seal hub adjacent a distal opening of the seal hub;
a cinched seal having a proximal end and a distal end, the cinched seal comprising:
a proximal flange at the proximal end of the cinched seal and a distal flange at the distal end of the cinched seal;
a proximal conical portion connected to and extending from the proximal flange and a proximal conical space within the proximal conical portion;
a distal conical portion connected to and extending from the distal flange and a distal conical space within the distal conical portion;
a center portion connected to the proximal conical portion and the distal conical portion, the center portion having a C-shaped cross-section;
a hole extending through the center portion and fluidly connected to the proximal conical space and the distal conical space; and
a proximal coil positioned on the proximal conical portion adjacent the center portion and a distal coil positioned on the distal conical portion adjacent the center portion;
wherein the hole in the center portion of the cinched seal is movable between a closed position for and an open position and the proximal coil and the distal coil provide a compressive force on the center portion directing the hole toward the closed position;
a ring adjacent the cinched seal;
a disc seal adjacent the ring; and
a cap adjacent the disc seal at a proximal opening of the seal hub.

10. The seal hub assembly of claim 9, wherein an outer diameter of the proximal flange and an outer diameter of the distal flange seal against an inner surface of the seal hub by an interference fit.

11. The seal hub assembly of claim 9, wherein the distal seal is a duckbill seal a distal end of the cinched seal is positioned adjacent and in contact with the duckbill seal, wherein the proximal end of the cinched seal is positioned adjacent and in contact with the ring.

12. A cross-plane seal comprising:
a disc at a distal end of the cross-plane seal, the disc including:
a first slit having a partial through-cut extending more than halfway through a thickness of the disc;
a second slit extending in a direction away from the first slit, the second slit having a partial through-cut extending more than halfway through the thickness of the disc;
an intersection formed by first slit and the second slit; and
an annular portion connected to and extending away from the disc.

13. The cross-plane seal of claim 12, wherein the disc has an outer diameter that is greater than an outer diameter of the annular portion,
wherein a cross-sectional profile of the cross-plane seal is U-shaped,
wherein a step is formed between the disc and the annular portion.

14. The cross-plane seal of claim 12, wherein the disc comprises:
a proximal face at a proximal side of the disc;
a distal face at a distal side of the disc; and
a plurality of cut outs in the distal face of the disc forming a raised X-shape on the distal face of the disc;
wherein the partial through-cut of the first slit is provided on the distal face of the disc and aligned with a first leg of the raised X-shape,
wherein the partial through-cut of the second slit is provided on the proximal face of the disc and aligned with a second leg of the raised X-shape, the through-cut extending more than halfway through the thickness of the disc, wherein the first slit is perpendicular to the second slit.

15. The cross-plane seal of claim 12, wherein the disc comprises:
a proximal face at a proximal side of the disc;
a distal face at a distal side of the disc;
wherein the first slit comprises a plurality of first curved slits and the second slit comprises a plurality of second curved slits, the partial through-cut of each of the plurality of first curved slits and the plurality of second curved slits extending into the proximal face of the disc,
wherein the plurality of first curved slits are offset from the plurality of second of curved slits,
wherein the plurality of first curved slits are spaced from each other, and the plurality of second curved slits are spaced from each other.

16. The cross-plane seal of claim 15, wherein the plurality of first curved slits are curved in a first direction, and the plurality of second curved slits are curved in a second direction opposite the first direction.

17. The cross-plane seal of claim 15, wherein the plurality of first curved slits each have an end at a center of the disc and the plurality of second curved slits each have an end at the center of the disc,
wherein the intersection is at the center of the disc.

18. The cross-plane seal of claim 12, wherein the disc comprises:
a proximal face at a proximal side of the disc;
a distal face at a distal side of the disc;
a star-shaped cut out having a star-shaped recess in the distal face;
wherein the partial through-cut of the first slit is provided on the distal face of the disc and aligned with arms of the star-shaped cut out, the partial through-cut extending more than halfway through a thickness of the disc;
wherein the partial through-cut of the second slit is provide the second face of the disc and aligned with arms of the star-shaped cut out, the partial through-cut extending more than halfway through the thickness of the disc, wherein the first slit is perpendicular to the second slit.

19. The cross-plane seal of claim 18, wherein the star-shaped cut out comprises a central region with eight rectangular arms evenly spaced around and extending from the central region, wherein the eight rectangular arms comprises the arms of the star-shaped cut out with which the first slit is aligned and the arms of the star-shaped cut out with which the second slit is aligned.

20. A seal hub assembly for a guide sheath, the seal hub assembly comprising:
a seal hub;
a seal stack received within the seal hub, the seal stack comprising:
a distal seal located within the seal hub adjacent a distal opening of the seal hub;
a cross-plane seal comprising:
a disc at a distal end of the cross-plane seal, the disc including:
a first slit having a partial through-cut extending more than halfway through a thickness of the disc;
a second slit extending in a direction away from the first slit, the second slit having a partial through-cut extending more than halfway through a thickness of the disc;
an intersection formed by first slit and the second slit;
an annular portion connected to and extending away from the disc; and
an outer diameter of the disc forming a sealing surface having an interference fit with an inner surface of the seal hub;
a ring adjacent the cross-plane seal;
a disc seal adjacent the ring; and
a cap adjacent the disc seal at a proximal opening of the seal hub.

21. The seal hub assembly of claim 20, wherein the distal seal is a duckbill seal, and a distal end of the cross-plane seal is positioned adjacent to in contact with the duckbill seal, and where the disc of the cross-plane seal positioned is between the duckbill seal and the ring.

22. The seal hub assembly of claim 20, wherein the distal seal is a cross-slit seal, and the cross-plane seal is positioned adjacent the cross-slit seal.

23. A seal hub assembly for a guide sheath, the seal hub assembly comprising:
a seal hub;
a seal stack received within the seal hub, the seal stack comprising:
a distal seal located within the seal hub adjacent a distal opening of the seal hub;
a medial seal adjacent the distal seal;
a ring adjacent the medial seal;
a disc seal adjacent the ring; and
a cap adjacent the disc seal at a proximal opening of the seal hub.

24. The seal hub assembly of claim 23, wherein the distal seal is a duckbill seal and the medial seal is a self-centering seal, where the ring is configured to fit between the duckbill seal and the self-centering seal,
wherein the self-centering seal comprises:
a disc comprising a plurality of raised curves extending from a distal end of the self-centering seal to a proximal end of the self-centering seal;
a central planar region connected to an inner diameter of the disc; and
a hole extending through the central planar region.

25. The seal hub assembly of claim 23, wherein the medial seal is a funnel seal comprising:
a disc at a proximal end of the funnel seal, a proximal face of the disc forming a proximal face of the funnel seal;
an annular portion connected to the disc; and
a funnel portion forming a funnel-shaped opening that extends from the proximal end to a distal end of the funnel seal.

26. The seal hub assembly of claim 23, wherein the medial seal is a collapsible seal comprising:
a first ring defining a proximal end of the collapsible seal;
a first membrane connected to the first ring;
a second ring connected to the first membrane such that the first membrane is between the first ring and the second ring;
a first wall extending across the first ring;
a second wall extending across the second ring;
a first hole extending through a center of the first wall; and
a second hole extending through a center of the second wall.
